(19) United States
(12) Patent Application Publication
(10) Pub. No.: US 2022/0187205 A1
(43) Pub. Date: Jun. 16, 2022

(54) SYSTEMS AND METHODS FOR CHIP REGENERATION

(71) Applicant: Quantum-Si Incorporated, Guilford, CT (US)

(72) Inventors: Guojun Chen, Sherborn, MA (US); Gerard Schmid, Guilford, CT (US); Todd Rearick, Cheshire, CT (US); Thomas Raymond Thurston, Guilford, CT (US); Haidong Huang, Madison, CT (US)

(21) Appl. No.: 17/551,085

(22) Filed: Dec. 14, 2021

Related U.S. Application Data
(60) Provisional application No. 63/125,847, filed on Dec. 15, 2020.

(51) Int. Cl. G01N 21/64; G01N 33/543

(52) U.S. Cl. CPC G01N 21/6428; G01N 2021/6439; G01N 33/54353

(57) ABSTRACT

Aspects of the disclosure relate to methods and systems for regenerating a sensor chip surface, including techniques for reuse of a single sensor chip in multiple sampling cycles by regenerating a surface of the sensor chip between successive sampling cycles. A method is provided for reusing an integrated device to process a sample, the sample being divided into a plurality of aliquots, the method comprising: loading a first aliquot of the plurality of aliquots into at least some of a plurality of chambers of the integrated device; sampling analytes of the first aliquot while the analytes are present in the at least some of the plurality of chambers; removing the first aliquot from the at least some of the plurality of chambers of the integrated device; and loading a second aliquot of the plurality of aliquots into the at least some of the plurality of chambers of the integrated device.

### CROSS-REFERENCE TO RELATED APPLICATIONS

This application claims priority under 35 U.S.C. § 119(e) to U.S. Provisional Patent Application No. 63/125,847, filed Dec. 15, 2020, which is hereby incorporated by reference in its entirety.

### FIELD OF THE DISCLOSURE

The present disclosure relates to integrated devices and related instruments that can perform massively-parallel analyses of samples by providing short optical pulses to tens of thousands of reaction chambers or more simultaneously and receiving fluorescent signals from the reaction chambers for sample analyses. The instruments may be useful for point-of-care genetic sequencing and for personalized medicine.

### BACKGROUND

Photodetectors are used to detect light in a variety of applications. Integrated photodetectors have been developed that produce an electrical signal indicative of the intensity of incident light. Integrated photodetectors for imaging applications include an array of pixels to detect the intensity of light received from across a scene. Examples of integrated photodetectors include charge coupled devices (CCDs) and Complementary Metal Oxide Semiconductor (CMOS) image sensors.

Instruments that are capable of massively-parallel analyses of biological or chemical samples are typically limited to laboratory settings because of several factors that can include their large size, lack of portability, requirement of a skilled technician to operate the instrument, power need, need for a controlled operating environment, and cost. When a sample is to be analyzed using such equipment, a common paradigm is to extract a sample at a point of care or in the field, send the sample to the lab and wait for results of the analysis. The wait time for results can range from hours to days

### SUMMARY OF THE DISCLOSURE

Aspects of the technology disclosed herein relate to methods of determining the identity or sequence of a sample containing a polypeptide molecule or a nucleic acid molecule. Further aspects relate to methods and devices for next-generation sequencing of samples, including single-molecule sequencing. Aspects of the disclosure relate to methods and systems for sequencing a polypeptide or a nucleic acid molecule using luminescent molecules such as luminescent labels (e.g., fluorophores).

Systems in accordance with the disclosure permit single-molecule analysis. Exemplary systems include an integrated device (also referred to as a chip) and an instrument configured to interface with the integrated device. The integrated device may include an array of pixels, where individual pixels include a sample well and at least one photodetector. The sample wells of the integrated device may be formed on or through a surface of the integrated device and be configured to receive a sample placed on the surface of the integrated device. Collectively, the sample wells may be formed as an array of sample wells. The plurality of sample wells may have a suitable size and shape such that at least a portion of the sample wells receive a single sample (e.g., a sample containing a single type of biomolecule). In some embodiments, the number of samples within a sample well may be distributed among the sample wells of the integrated device such that some sample wells contain one sample while others contain zero, two or more samples.

Some embodiments of the disclosure provide for a method for reusing an integrated device to process a sample that is divided into a plurality of aliquots. In various embodiments, the sample comprises analytes and the analytes comprise a biomolecule comprising one or more luminescently labeled molecules, the method comprising: loading a first aliquot of the plurality of aliquots into at least some of a plurality of chambers of the integrated device; sampling analytes of the first aliquot while the analytes are present in the at least some of the plurality of chambers, wherein the step of sampling comprises determining a luminescent lifetime, a luminescent intensity, a wavelength, a pulse duration, and/or an interpulse duration of a signal emitted by the one or more luminescently labeled molecules; removing the first aliquot from the at least some of the plurality of chambers of the integrated device; and loading a second aliquot of the plurality of aliquots into the at least some of the plurality of chambers of the integrated device. While the term aliquot may, in some embodiments, refer to portions of a same sample, in other embodiments, first and second aliquots may comprise portions of first and second samples derived from different sources.

Some embodiments provide for a method for reusing an integrated device to process a sample, the sample comprising analytes, the analytes comprising a biomolecule comprising one or more luminescently labeled molecules, the method comprising: loading at least a portion of the sample into a plurality of chambers of the integrated device; sampling analytes of the at least the portion of the sample while the analytes are present in the plurality of chambers, wherein the step of sampling comprises collecting a signal emitted by the one or more luminescently labeled molecules indicative of a luminescent lifetime, a luminescent intensity, a wavelength, a pulse duration, and/or an interpulse duration; and removing the at least the portion of the sample from the plurality of chambers, wherein the removing comprises: disrupting a covalent bond between respective coating molecules bound to a surface of the plurality of chambers and coupling moieties, the analytes of the at least the portion of the sample being bound to the coupling moieties.

Some embodiments provide for a method for determining whether a sample is present in one or more chambers of an integrated device, the method comprising: loading at least a portion of the sample into the one or more chambers of the integrated device; removing the at least the portion of the sample from the one or more chambers of the integrated device; delivering excitation light to the one or more chambers of the integrated device; collecting signals emitted from the plurality of chambers in response to the excitation light at a photodetection region of the integrated device; and determining, based on the signals, whether at least some of the at least the portion of the sample is present in the one or more chambers of the integrated device.

Some embodiments provide for a method for determining whether to continue processing a sample with an integrated device, the sample being divided into a plurality of aliquots, the method comprising: sampling analytes of a first aliquot of the plurality of aliquots while the analytes are present in one or more chambers of the integrated device, the sampling comprising: exciting the analytes of the first aliquot with excitation light delivered from at least one light source; and collecting, at a photodetection region of the integrated device, signals emitted from the analytes of the first aliquot when excited by the light delivered from the at least one light source; and determining, based on the signals emitted from the analytes of the first aliquot, to load a second aliquot of the plurality of aliquots into the one or more chambers of the integrated device; and loading a second aliquot of the plurality of aliquots into the one or more chambers of the integrated device.

Some embodiments provide for a reusable device for processing a sample, the sample being divided into a plurality of aliquots, the device comprising: a plurality of reaction chambers for receiving a first aliquot of the plurality of aliquots, wherein surfaces of the plurality of reaction chambers are coated with coating molecules configured to bind to coupling moieties bonded to analytes of the first aliquot; a photodetection region configured to receive signals emitted from the plurality of chambers in response to excitation light being delivered to the plurality of chambers; at least one processor configured to determine, based on the signals received by the photodetection region, whether the plurality of chambers contain at least a portion of the analytes of the first aliquot.

Some embodiments provide for a method, comprising: loading a first sample into a plurality of chambers of a reusable chip, the reusable chip having a plurality of waveguides for directing excitation light received from an instrument comprising at least one light source to the plurality of chambers, and a plurality of photodetection regions for receiving light emitted from the first sample; delivering excitation light from the at least one light source of the instrument to the plurality of chambers of the reusable chip; performing sequencing of analytes of the first sample; removing the first sample from the plurality of chambers of the reusable chip; and loading a second sample into the plurality of chambers of the reusable chip.

Some embodiments provide for a device, comprising: a reusable chip, for use with an instrument comprising at least one light source, the reusable chip comprising: a plurality of chambers for receiving a first sample; a plurality of waveguides to direct excitation light from the at least one light source to the plurality of chambers; and a plurality of photodetection regions for receiving light emitted from the first sample, wherein the reusable chip is configured such that the first sample can be removed from plurality of chambers and a second sample can be received in the plurality of chambers after the first sample is removed.

Some embodiments provide for devices and methods for reusing chips through regeneration of chip surfaces, wherein the devices and methods are suitable for massively-parallel analyses of biological or chemical samples. In some embodiments, these devices and methods are further suitable for automated, massively-parallel analyses.

### BRIEF DESCRIPTION OF DRAWINGS

The features and advantages of the present disclosure will become more apparent from the detailed description set forth below when taken in conjunction with the drawings. When describing embodiments in reference to the drawings, directional references (“above,” “below,” “top,” “bottom,” “left,” “right,” “horizontal,” “vertical,” etc.) may be used. Such references are intended merely as an aid to the reader viewing the drawings in a normal orientation. These directional references are not intended to describe a preferred or only orientation of features of an embodied device. A device may be embodied using other orientations.

FIG. 1A is a block diagram of an integrated device and an instrument, according to some embodiments.

FIG. 1B is a schematic of an integrated device, according to some embodiments.

FIG. 1C is a schematic of a pixel of an integrated device, according to some embodiments.

FIG. 1D is a circuit diagram of the pixel of FIG. 1C, according to some embodiments.

FIG. 1E is a top view of the pixel of FIG. 1C, according to some embodiments.

FIG. 1F is a plan view of the pixel of FIGS. 1C and 1D, according to some embodiments.

FIG. 1G is a schematic of an alternative pixel of an integrated device, according to some embodiments.

FIG. 1H is a circuit diagram of the pixel of FIG. 1G, according to some embodiments.

FIG. 1I is a plan view of the pixel of FIGS. 1C and 1D, according to some embodiments.

FIG. 2A is an example diagram of an analyte having been immobilized in a chamber of an integrated device, according to some embodiments.

FIG. 2B is an exemplary process for reusing an integrated device of FIG. 1A to process multiple samples, according to some embodiments.

FIG. 3A is an exemplary process for regenerating the surface of a chamber of an exemplary integrated device of FIG. 1A, according to some embodiments.

FIG. 3B is another exemplary process for regenerating the surface of a chamber of the integrated device of FIG. 1A, according to some embodiments.

FIG. 3C is another exemplary process for regenerating the surface of a chamber of the integrated device of FIG. 1A, according to some embodiments.

FIG. 3D is another exemplary process for regenerating the surface of a chamber of the integrated device of FIG. 1A, according to some embodiments.

FIGS. 4A-4D illustrate example graphs depicting the presence of analyte obtained during a process of chip loading and regeneration, according to some embodiments.

FIG. 4E illustrates spectra of samples soaked in an exemplary regeneration solution under different conditions, according to some embodiments.

FIG. 4F illustrates example graphs illustrating signals acquired from reaction chambers of an exemplary integrated device between regeneration and loading steps, according to some embodiments.

FIGS. 4G-4I illustrate additional example graphs illustrating signals acquired from reaction chambers of an exemplary integrated device between regeneration and loading steps, according to some embodiments.

FIG. 4J illustrates additional example graphs illustrating signals acquired from reaction chambers of an exemplary integrated device between regeneration and loading steps, according to some embodiments.

FIG. 5A is a flow chart depicting an exemplary process for determining whether a sample is present in a chamber of the integrated device of FIG. 1A, according to some embodiments.

FIG. 5B is a flow chart depicting an exemplary process for determining whether to continue processing a sample with the integrated device of FIG. 1A, according to some embodiments.

FIG. 6A is a block diagram of an analytical instrument that includes a compact mode-locked laser module, according to some embodiments.

FIG. 6B is an exemplary compact mode-locked laser module incorporated into an analytical instrument, according to some embodiments.

FIG. 6C illustrates an example of parallel reaction chambers that can be excited optically by a pulsed laser via one or more waveguides, according to some embodiments.

FIG. 6D illustrates further details of an integrated device reaction chamber, optical waveguide, and time-binning photodetector, according to some embodiments.

FIGS. 7A and 7B illustrate the results of an experiment evaluating the sequencing accuracy of a DNA analyte, according to an exemplary method of regenerating a chip in some embodiments of the disclosure. FIG. 7A is a line plot showing the mean read lengths among signals acquired from three sequential runs (two chip regenerations) on an exemplary integrated device between regeneration and loading steps. FIG. 7B shows line plots depicting the mean accuracy, maximum accuracy, and “alignment” of each of these three runs.

FIGS. 8A-8H illustrate the signal readout results of an experiment evaluating the sequencing accuracy over three read lengths of a peptide analyte, according to an exemplary method of regenerating a chip in some embodiments of the disclosure. This experiment was repeated on two CMOS chips.

FIG. 9 illustrates an example process for calibrating a system comprising an integrated device using photobleaching information, according to some embodiments.

FIG. 10-1 illustrates a dye-labeled sample attached to a surface, according to some embodiments.

FIG. 10-2 illustrates a dye-labeled sample attached in a reaction chamber, according to some embodiments.

FIG. 11 illustrates an example graph illustrating light source excitation power over time, according to some embodiments.

FIG. 12-1 illustrates an example graph illustrating measured signal from a reference dye over time, according to some embodiments.

FIG. 12-2 illustrates example histograms of measured bleaching time and dye intensity for a collection of single molecules in reaction chambers, according to some embodiments.

FIG. 13 illustrates a sequencing reaction in a reaction chamber, according to some embodiments.

FIG. 14 illustrates an example process for quantifying loading of one or more reaction chambers, according to some embodiments.

FIG. 15-1 illustrates an example trace from a chip loading process that represents a single loaded well, according to some embodiments.

FIG. 15-2 illustrates an example trace from a chip loading process that represents a double loaded well, according to some embodiments.

FIG. 15-3 illustrates an example trace from a chip loading process that represents a multi-loaded well, according to some embodiments.

FIG. 16 illustrates a periodic pulsing pattern of fluorescent molecules reversibly bound to a biomolecule, according to some embodiments.

FIG. 17 illustrates an example heatmap of reaction chambers illustrating percent loading, according to some embodiments.

### DETAILED DESCRIPTION

I. Introduction

Aspects of the present disclosure relate to methods and systems for regenerating a sensor chip surface, the sensor chip forming all or a portion of an integrated device. In particular, according to some embodiments, there are provided techniques for reuse of a single sensor chip in multiple sampling cycles by regenerating a surface of the sensor chip between successive sampling cycles.

An integrated device may comprise a sensor chip having a plurality of reaction chambers each for receiving a sample. Sample data may be obtained by exciting portions of the samples disposed in the plurality of reaction chambers with excitation light, and collecting signals emitted from the samples in response to the excitation with a photodetector. In order to perform sampling, portions of the samples present in the reaction chambers are immobilized. For example, a surface of each reaction chamber may be coated with a molecule, such as biotin, and the sample portion may be functionalized with a biotin-bonding protein, such as streptavidin to immobilize the portion of the sample in the reaction chamber. Typically, the sensor chip is disposed after performing sampling.

In many applications, it is desirable to detect a large number of analyte species in a sample. However, integrated devices which perform sampling are limited in the number of pixels, and therefore reaction chambers, available for receiving analyte species of a sample. Often the number of reaction chambers on an integrated device is less than the number of analyte species desired to be sampled. In addition, although the sample may be diluted to obtain a titration which maximizes the number of reaction chambers receiving a single analyte species, many reaction chambers still do not produce useful measurements because they either contain no analytes or contain two or more analyte species such that obtaining a desirable single-analyte measurement is not possible. Thus, a single integrated device is typically unable to process a sample containing a large number of species.

One technique for sampling a large number of analyte species in a single sample is to divide the sample into subdivisions, referred to herein as aliquots, and to process the aliquots individually, as separate experiments. For example, each aliquot of the sample may be processed separately by a respective integrated device. However, such a technique is expensive as it requires the use of multiple integrated devices.

The inventors have recognized that it would be advantageous to reuse a single integrated device for performing multiple experiments on a single sample and/or multiple samples before disposing of the integrated device. That way, a sample comprising a large number of analyte species divided into multiple aliquots may be processed in consecutive experiments using a single integrated device. According to conventional techniques, a sample may be processed by (1) coating a chip surface with a molecule, such as biotin; (2) loading a first aliquot of a sample containing an analyte bound to a coupling moiety for binding to the coating, such as a streptavidin and allowing the labeled analyte to be bound to the surface of the chip; (3) rinsing unbound species; and (4) performing a measurement (e.g., by exciting the analytes bound to the surface of the reaction chambers and collecting signals emitted from the excited analytes). As described herein, diluting the sample to a titration which maximizes the number of reaction chambers receiving only a single species of analyte also results in a number of reaction chambers which contain no analytes. Thus, it is possible to reload the integrated device with a second aliquot of the sample to facilitate measurement of analytes in reaction chambers which remain unoccupied. However, the occupation of reaction chambers from the initial loading of the first aliquot may inhibit or interfere with measurements of analytes in the second aliquot.

Thus, the inventors have developed techniques for regenerating a sensor chip surface that has previously been used. In particular, the techniques described herein relate to a method for unloading an analyte species that has been previously bound to the surface of a sensor chip via a biotin-streptavidin bond or other interaction. For example, example techniques for unloading the sensor chip include (a) disrupting the bonded anchoring (e.g., of streptavidin and biotin); (b) stripping the molecule (e.g., biotin) coating from the surface of the sensor chip; (c) enzymatically digesting the sample analyte in occupied reaction chambers; and (d) melting a bond between the sample analyte and a coupling moiety (e.g., streptavidin, biotin). Subsequent to regenerating the sensor chip surface, the integrated device may be reloaded with a second aliquot of the sample. Such techniques allow for processing an entire sample comprising multiple aliquots despite the large number of analyte species present in the sample.

The inventors have further recognized that in reusing the integrated device for multiple experiments, components of the integrated device may deteriorate over the extended use of the sensor chip. Accordingly, some aspects of the technology described herein relate to techniques for increasing the longevity of use of the integrated device.

The techniques described herein for regeneration of a sensor chip surface of an integrated device may be used in combination with DNA/RNA and/or protein sequencing applications, in some embodiments.

It should be appreciated that integrated devices described herein may incorporate any or all techniques described herein alone or in combination. Additionally, other integrated devices besides those described herein may employ the methods and structures described.

Surface Functionalization or Coating

In certain embodiments, techniques described herein can be used to coat (or functionalize, or confine) a molecule of interest to a desired region of a sample well (also referred to herein as a reaction chamber). In some embodiments, a sample well occupies a volume or space defined by an opening formed at a surface of an integrated device which extends through a first layer and into a second layer of the integrated device to a bottom surface distal to the opening. In some embodiments, the exposed surfaces of the first layer and second layer disposed between the opening and the bottom surface of the sample well may be referred to as sidewalls which further define the volume or space occupied by the sample well.

In embodiments when one or more analytes (e.g., a DNA analyte or a peptide analyte) is immobilized on the bottom surface, it may be desirable to coat the bottom surface to allow for attachment of one or more molecules or complexes. In some embodiments, the bottom surface is coated with a coating molecule. In some embodiments, the coating molecule is an avidin protein. Avidin proteins are biotin-binding proteins, generally having a biotin binding site at each of four subunits of the avidin protein. Avidin proteins include, for example, avidin, streptavidin, traptavidin, tamavidin, bradavidin, xenavidin, and homologs and variants thereof. In some embodiments, the avidin protein is streptavidin. The multivalency of avidin protein can allow for various linkage configurations, as each of the four binding sites are independently capable of binding a biotin molecule.

In certain embodiments, the coating molecule is one of biotin, streptavidin, and a biotin-streptavidin complex. In some embodiments, the coating molecule is an analog of biotin or an analog of streptavidin. In some embodiments, the coating molecule is a bis-biotin. In some embodiments, the coating molecule participates in an interaction with a coupling moiety (such as a streptavidin moiety), such as a bis-biotin-streptavidin bond.

In some embodiments, the coating molecule is a silane comprising an alkyl chain. In some embodiments, the coating molecule is a silane comprising an optionally substituted alkyl chain. In some embodiments, the coating molecule is a silane comprising a poly(ethylene glycol) chain. In some embodiments, the coating molecule is a silane comprising a coupling moiety. For example, the coupling moiety may comprise chemical moieties, such as amine groups, carboxyl groups, hydroxyl groups, sulfhydryl groups, metals, chelators, and the like. Alternatively, the coupling moiety may include specific binding elements, such as biotin, avidin, streptavidin, neutravidin, lectins, SNAP-TAGs® (self-labeling protein tags), associative or binding peptides or proteins, antibodies or antibody fragments, nucleic acids or nucleic acid analogs, or the like. The coupling moiety may comprise a methyl ether group. Additionally, or alternatively, an intermediate binding agent and/or copolymer may be used to couple an additional group that is used to couple or bind with the molecule of interest, which may, in some cases include both chemical functional groups and specific binding elements. By way of example, a coupling moiety, e.g., biotin, may be deposited upon a substrate surface and selectively activated in a given area. An intermediate binding agent, e.g., streptavidin, may then be coupled to the biotin coupling moiety. The analyte of interest, which in an exemplary workflow may itself be biotinylated, is then coupled to the streptavidin.

The chemical structure for biotin is reproduced below:

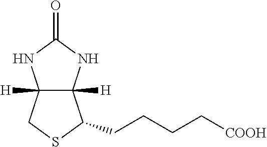 [SMILES: O=C(O)CCCC[C@@H]1SC[C@@H]2NC(=O)N[C@@H]21]

Streptavidin contains four biotin binding sites. Even after a streptavidin is coated onto the bottom surface of the sample well, three additional binding sites remain for conjugation to a biotin or a biotin-conjugated polymer.

Examples of coating molecules include, without limitation, a trans-cyclooctene (TCO) moiety, a tetrazine moiety, an azide moiety, an alkyne moiety, an aldehyde moiety, a methyl ether moiety, an isocyanate moiety, an N-hydroxysuccinimide moiety, a thiol moiety, an alkene moiety, a dibenzocyclooctyl moiety, a bicyclononyne moiety, and a thiamine pyrophosphate moiety. Examples of coating molecules that comprise a covalent coupling moiety include, without limitation, azide-silanes and azide-organosilanes, such as azide-PEG-silane (e.g., azide-PEG₃-silane, azide-PEG₅-silane) and azide-alkylsilane (e.g., azide-C₁₁-silane). In some embodiments, the coupling moiety is a non-covalent coupling moiety.

Examples of coupling moieties include, without limitation, a biotin moiety, an avidin protein, a streptavidin protein, a lectin protein, a SNAP-tag, a methyl ether, and a biotin-streptavidin complex (e.g., a bis-biotin-streptavidin complex). Exemplary biotin moieties include mono-biotin (free biotin) and bis-biotin. In some embodiments, the coupling moiety comprises an avidin protein.

In some embodiments, the methods further comprise coating the bottom surface of the sample well with multiple coating molecules, such as a first coating molecule and a second coating molecule. The second coating molecule may comprise a biotin moiety, an avidin protein, a streptavidin protein, an azide moiety, an alkyne moiety, a ketone moiety, a hydroxylamine moiety. In some embodiments, the second coating molecule is a biotin moiety or streptavidin protein. In some embodiments, the first coating molecule and the second coating molecule are the same. In some embodiments, the first coating molecule and the second coating molecule are the different. In some embodiments, the first coating molecule and the second coating molecule are both biotin. In some embodiments, the first coating molecule and the second coating molecule are both streptavidin. In some embodiments, the second coating molecule is an azide moiety. In some embodiments, the analyte is immobilized to a first coating agent comprising a biotin moiety, while a second coupling moiety comprising an azide moiety is unconjugated.

As used herein, in some embodiments, a “surface” refers to a surface of a substrate or solid support containing one or more sample wells. In some embodiments, a solid support refers to a material, layer, or other structure having a surface, such as a receiving surface, that is capable of supporting a deposited material, such as a functionalized peptide or DNA polymerase as described herein. In some embodiments, a receiving surface of a substrate may optionally have one or more features, including nanoscale or microscale recessed features such as an array of sample wells. In some embodiments, an array is a planar arrangement of elements such as sensors or sample wells. An array may be one or two dimensional. A one dimensional array is an array having one column or row of elements in the first dimension and a plurality of columns or rows in the second dimension. The number of columns or rows in the first and second dimensions may or may not be the same. In some embodiments, the array may include, for example, 10², 10³, 10⁴, 10⁵, 10⁶, or 10⁷ sample wells.

II. Integrated Device Overview

The techniques described herein may be used to regenerate the surface of a sensor chip of an integrated device. The integrated device may facilitate providing excitation light from one or more excitation sources located in one exemplary embodiment within an instrument to a reaction chamber containing a sample within the integrated device. The excitation light may be directed at least in part by elements such as waveguides of the integrated device towards one or more pixels that contain the reaction chambers to illuminate an illumination region within the reaction chamber. A sample disposed in a reaction chamber, or a reaction component attached to the sample (such as a fluorescent label, for example), may emit emission light when located within the illumination region of the reaction chamber in response to being illuminated by the excitation light. In some embodiments, the one or more excitation sources are located within an instrument into which the integrated device is inserted and cooperates, both of which are part of an overall system.

While the integrated device reuse concepts are described with respect to an exemplary system embodiment in which an instrument into which the integrated device is inserted and cooperates includes at least one laser light source, and the integrated device includes waveguides for directing light and pixels having reaction chambers and detection regions, the innovation is not so limited. Other instrument designs and integrated device designs are envisioned, including ones where the light sources are not located within the instrument, and/or where additional optical components are located within the instrument (e.g., “off-chip”) rather than the integrated device.

Emission light emitted from one or more reaction chambers (e.g., at least two reaction chambers, in some embodiments), may be detected by one or more photodetectors within a pixel of the integrated device. As described herein, the integrated device may be configured having multiple pixels (e.g., an array of pixels), and thus, may have multiple reaction chambers and corresponding photodetectors. Characteristics of the detected emission light may provide an indication for identifying the label associated with the emission light. Such characteristics may include any suitable type of characteristic, including an arrival time of photons detected by the photodetector, an amount of photons accumulated over time by a photodetector, and/or a distribution of photons across two or more photodetectors. In some embodiments, a photodetector may have a configuration that allows for detection of one or more characteristics associated with the emission light, such as timing characteristics (e.g., luminescent lifetime, pulse duration, interpulse duration), wavelength, and/or intensity. As one example, one or more photodetectors may detect a distribution of photon arrival times after a pulse of excitation light propagates through the integrated device, and the distribution of arrival times may provide an indication of a timing characteristic of the emission light (e.g., a proxy for luminescent lifetime, pulse duration, and/or interpulse duration). Such information may be used in techniques for detection and/or identification of molecules in a sample, for example, including those described in U.S. patent application Ser. No. 16/686,028 titled “METHODS AND COMPOSITIONS FOR PROTEIN SEQUENCING,” filed Nov. 15, 2019 under Attorney Docket No. R0708.70042US02, PCT Application No. PCT/US19/61831 titled “METHODS AND COMPOSITIONS FOR PROTEIN SEQUENCING,” filed Nov. 15, 2019 under Attorney Docket No. R0708.70042WO00, U.S. patent application Ser. No. 17/190,331 titled “INTEGRATED SENSOR FOR MULTI-DIMENSIONAL SIGNAL ANALYSIS,” filed Mar. 2, 2021 under Attorney Docket No. R0708.70090US01, U.S. patent application Ser. No. 15/600,979 titled “LABELED NUCLEOTIDE COMBINATIONS AND METHODS FOR NUCLEIC ACID SEQUENCING,” filed May 22, 2017 under Attorney Docket No. R0708.70018US02, and U.S. patent application Ser. No. 15/161,125 titled “METHODS FOR NUCLEIC ACID SEQUENCING,” filed May 20, 2016 under Attorney Docket No. R0708.70020US00, each of which are incorporated by reference in their entireties. In some embodiments, one or more photodetectors provide an indication of a probability of emission light emitted by the fluorescent labels (e.g., luminescent intensity). In some embodiments, one or more photodetectors may be sized and arranged to capture a spatial distribution of the emission light (e.g., wavelength). Output signals from the one or more photodetectors may be used to distinguish a fluorescent label from among a plurality of labels, where the plurality of labels may be used to identify a sample or it structure, as described herein.

As used herein, an “integrated device” is a device capable of interfacing with a base instrument. In some embodiments, an integrated device may comprise one or more sample wells and/or sensors. In some embodiments, an integrated device may be capable of interfacing with a base instrument that emits or detects light. In such embodiments, the integrated device may comprise one or more sample wells, each of which includes a waveguide. The “integrated device” may be referred to herein as a microchip (or “chip”), such as a CMOS chip. An exemplary integrated device is a CMOS chip containing an array of pixels, where individual pixels include a sample well and photodetector.

In some aspects, the disclosure provides an integrated device comprising a substrate comprising a sample well having a metal oxide surface and a silica surface. In some embodiments, the integrated device further comprises a coating layer on the metal oxide surface formed by an amphipathic reagent that comprises a hydrophilic head group and a hydrophobic tail group. In some embodiments, the integrated device further comprises a functionalizing agent bound to the silica surface, wherein the functionalizing agent comprises a coupling moiety. In some embodiments, the substrate comprises an array of sample wells, each sample well having a metal oxide surface and a silica surface. In some embodiments, the array is a microfabricated microarray. In some embodiments, the substrate contains 128,000 sample wells. In some embodiments, the sample well comprises a top aperture formed at a surface of the substrate and a bottom surface distal to the surface of the substrate. In some embodiments, the bottom surface is comprised by the silica surface.

In some embodiments, a sample well comprises an immobilization region that may be a discrete region of a surface of a substrate that binds an analyte of interest, such as a bottom surface of a sample well having a polypeptide or a nucleic acid coupled to such surface. In some embodiments, sample wells comprise hollows or wells having defined shapes and volumes which are manufactured into a substrate or device. Sample wells can be fabricated using techniques described in the art, for example, as disclosed in U.S. application Ser. No. 16/555,902, which is incorporated herein by reference in its entirety.

In some embodiments, the sample well is formed by a bottom surface comprising a non-metallic layer and side wall surfaces comprising a metallic layer. In some embodiments, non-metallic layer comprises a transparent layer (e.g., glass, silica). In some embodiments, metallic layer comprises a metal oxide surface (e.g., titanium dioxide). In some embodiments, metallic layer comprises a passivation coating (e.g., a phosphorus-containing layer, such as an organophosphonate layer). The bottom surface may comprise a non-metallic layer that comprises a functional moiety. In some embodiments, the sample well comprises a top surface that contains an aluminum film, a titanium nitride (tinite) film, or both. The top surface may further comprise a selective surface chemistry coating, such as a coating known in the art. In some embodiments, the integrated device is configured to interface with a next-generation sequencing instrument, such as a benchtop next-generation sequencing (NGS) instrument.

An integrated device of the type described herein may comprise one or more sample wells configured to receive molecules of interest therein. In some embodiments, a sample well receives a molecule of interest that may be disposed on a surface of the sample well, such as a bottom surface. In some embodiments, a sample well is formed within an integrated device, wherein the bottom surface of the sample well is distal to the surface of the integrated device into which it is formed. In some embodiments, the bottom surface on which the molecule of interest is to be disposed may have a distance from a waveguide that is configured to excite the molecule of interest with a desired level of excitation energy. In some embodiments, the sample well may be positioned, with respect to a waveguide, such that an evanescent field of an optical mode propagating along the waveguide overlaps with the molecule of interest.

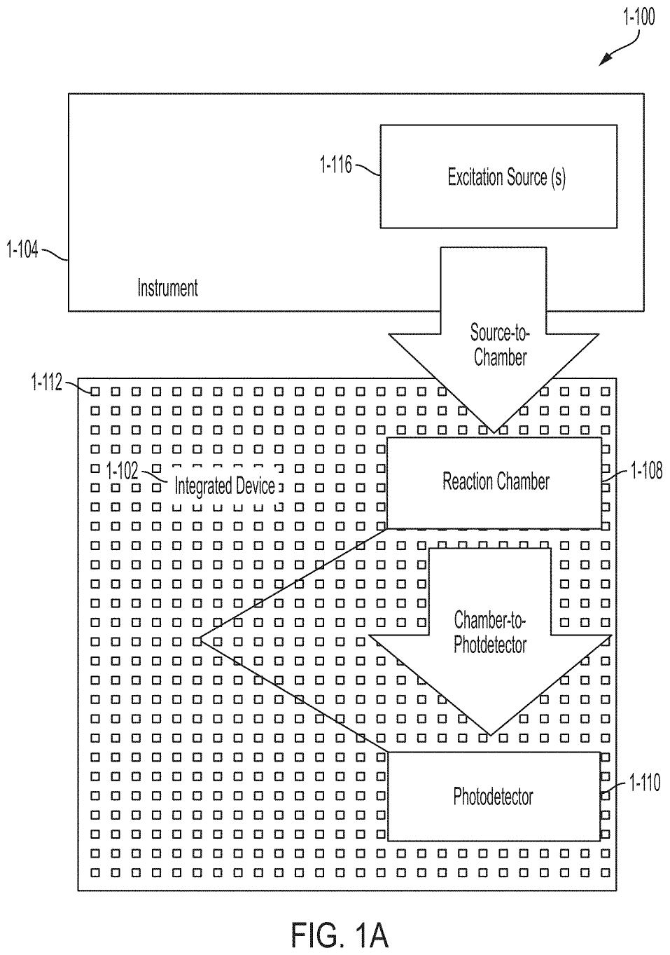

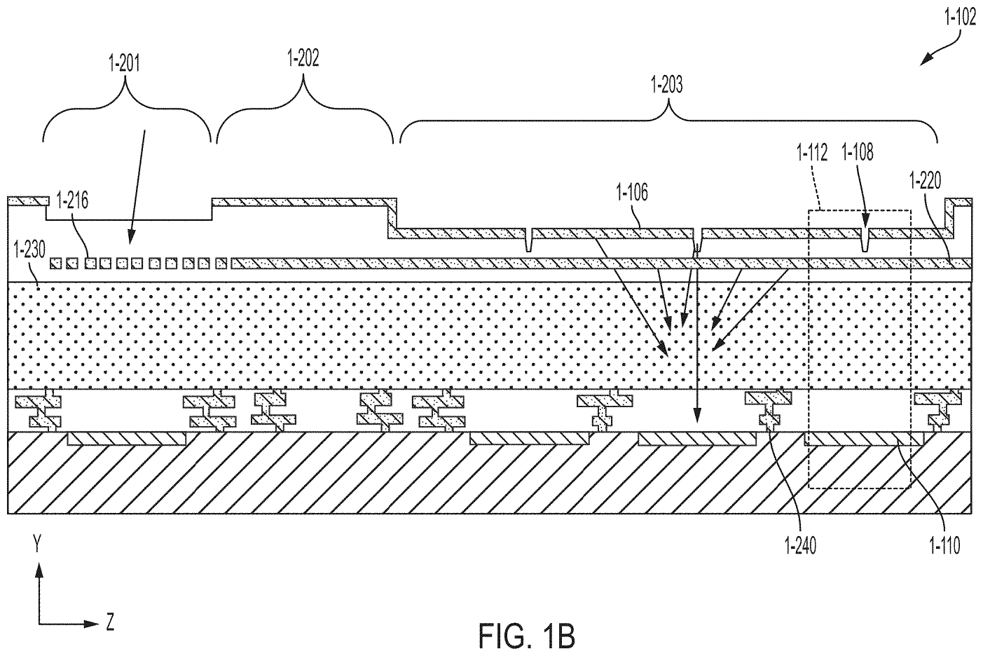

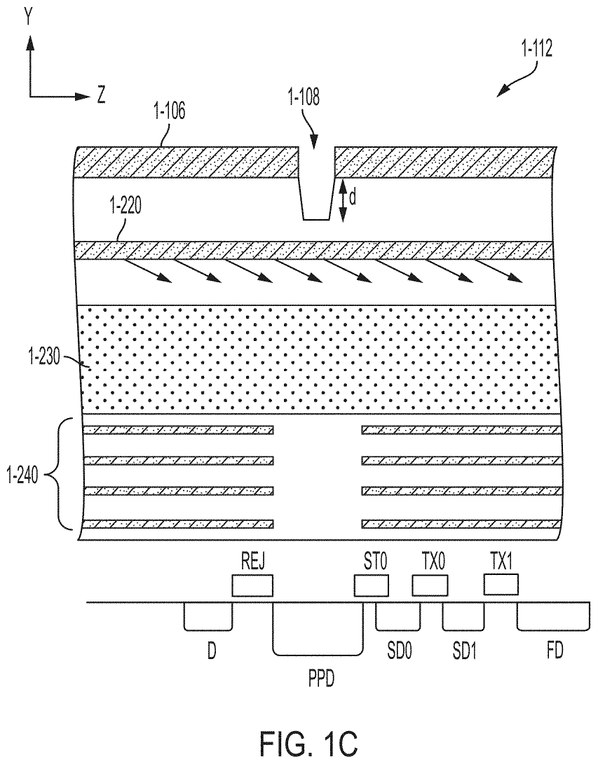

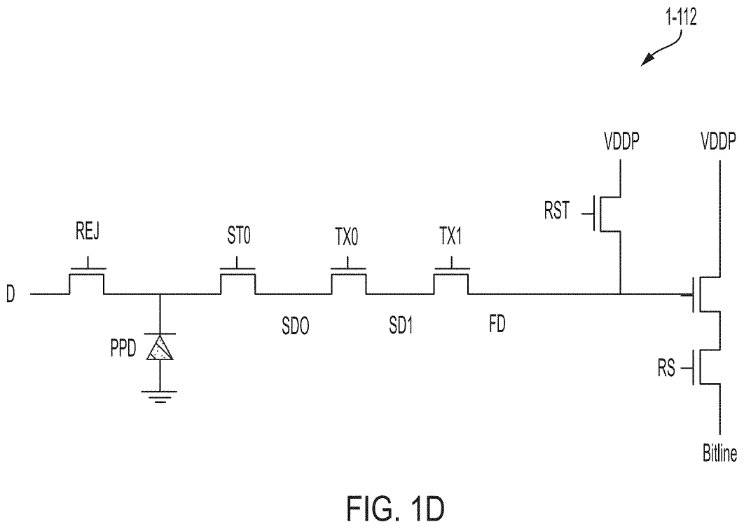

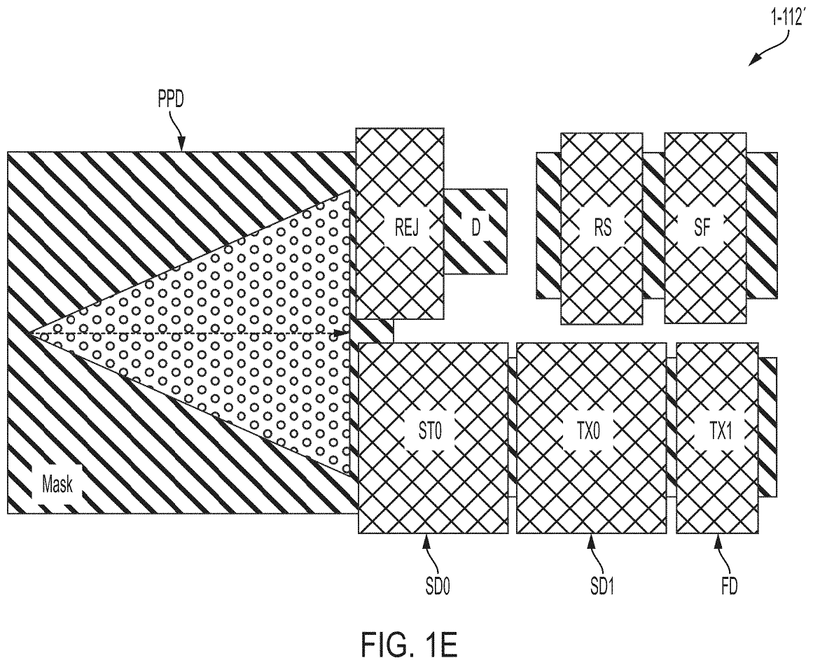

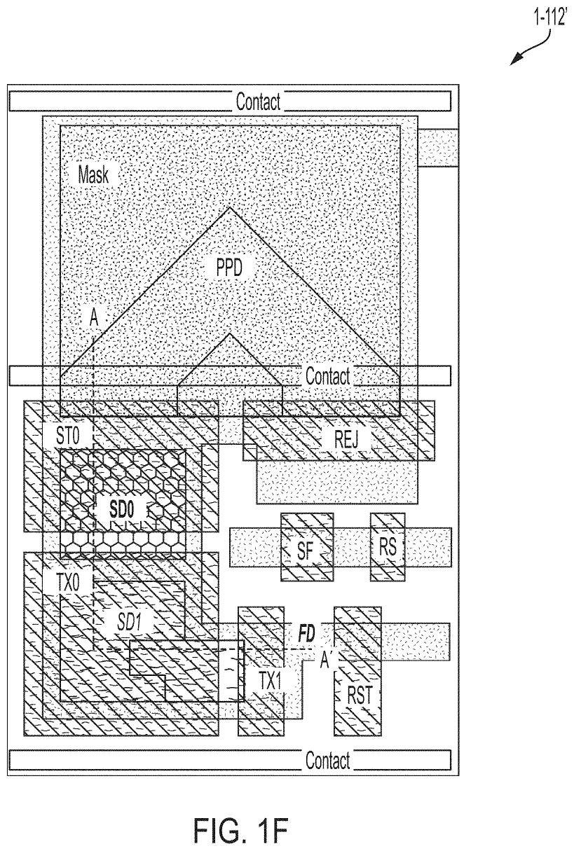

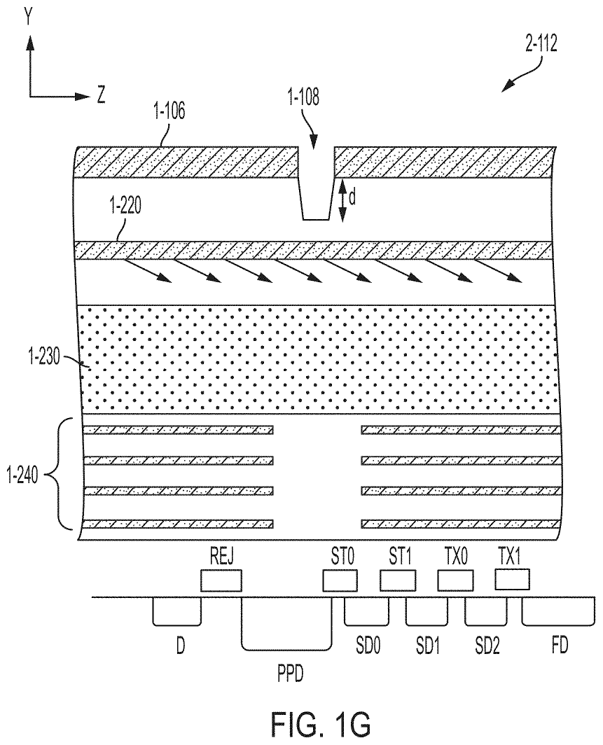

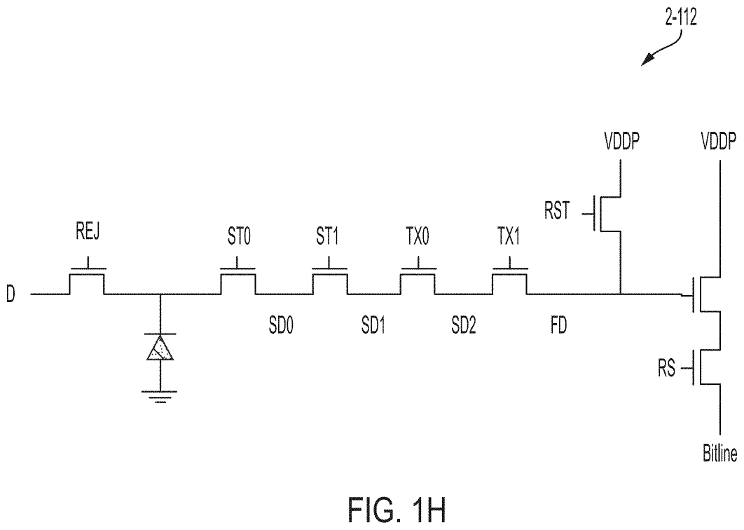

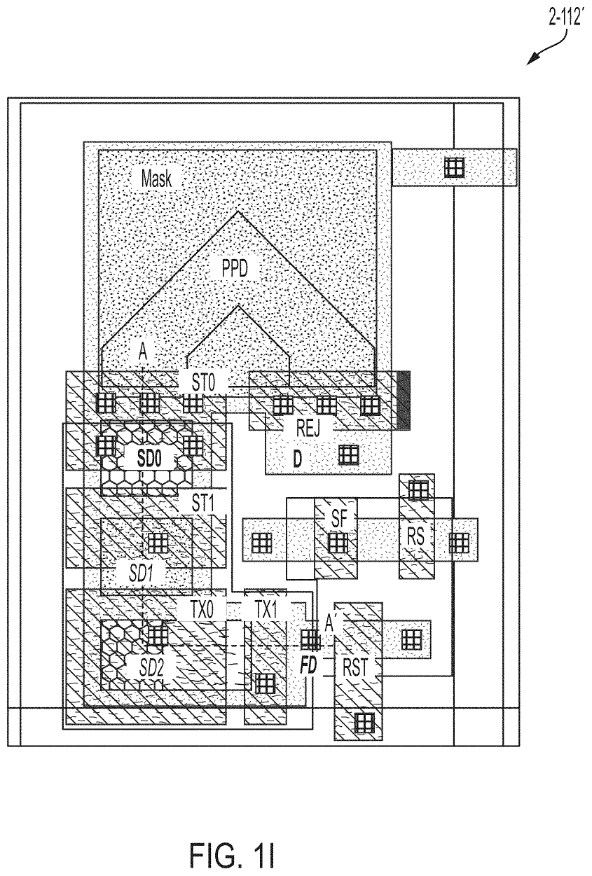

For example, a schematic overview of an exemplary system 1-100 is illustrated in FIG. 1A. The system comprises both an integrated device 1-102 that interfaces with an instrument 1-104. In some embodiments, instrument 1-104 may include one or more excitation sources 1-116 integrated as part of instrument 1-104. In some embodiments, an excitation source may be external to both instrument 1-104 and integrated device 1-102, and instrument 1-104 may be configured to receive excitation light from the excitation source and direct excitation light to the integrated device. The integrated device may interface with the instrument using any suitable socket for receiving the integrated device and holding it in precise optical alignment with the excitation source. The excitation source 1-116 may be configured to provide excitation light to the integrated device 1-102. As illustrated schematically in FIG. 1A, the integrated device 1-102 has a plurality of pixels 1-112, where at least a portion of pixels may perform independent analysis of a sample of interest. Such pixels 1-112 may be referred to as “passive source pixels” since a pixel receives excitation light from a source 1-116 separate from the pixel, where excitation light from the source excites some or all of the pixels 1-112. Excitation source 1-116 may be any suitable light source. Examples of suitable excitation sources are described in U.S. patent application Ser. No. 14/821,688, filed Aug. 7, 2015, titled “INTEGRATED DEVICE FOR PROBING, DETECTING AND ANALYZING MOLECULES” under Attorney Docket Number R0708.70004US02, which is incorporated by reference in its entirety. In some embodiments, excitation source 1-116 includes multiple excitation sources that are combined to deliver excitation light to integrated device 1-102. The multiple excitation sources may be configured to produce multiple excitation energies or wavelengths.

Referring to FIG. 1B, a pixel 1-112 has a reaction chamber 1-108 configured to receive a at least one sample of interest and a photodetector 1-110 for detecting emission light emitted from the reaction chamber in response to illuminating the sample and at least a portion of the reaction chamber 1-108 with excitation light provided by the excitation source 1-116. In some embodiments, reaction chamber 1-108 may retain the sample in proximity to a surface of integrated device 1-102, which may ease delivery of excitation light to the sample and detection of emission light from the sample or a reaction component (e.g., a luminescent label). As shown in the illustrated embodiment of FIG. 1B, the reaction chamber 1-108 and the photodetector 1-110 have a one-to-one correspondence. In some embodiments, as described herein, each pixel may comprise multiple reaction chambers per photodetector.

Optical elements for coupling excitation light from excitation light source 1-116 to integrated device 1-102 and guiding excitation light to the reaction chamber 1-108 may be located on one or both of the integrated device 1-102 and the instrument 1-104. Source-to-chamber optical elements may comprise one or more grating couplers located on integrated device 1-102 to couple excitation light to the integrated device and waveguides to deliver excitation light from instrument 1-104 to reaction chambers in pixels 1-112. One or more optical splitter elements may be positioned between a grating coupler and the waveguides. The optical splitter may couple excitation light from the grating coupler and deliver excitation light to at least one of the waveguides. In some embodiments, the optical splitter may have a configuration that allows for delivery of excitation light to be substantially uniform across all the waveguides such that each of the waveguides receives a substantially similar amount of excitation light. Such embodiments may improve performance of the integrated device by improving the uniformity of excitation light received by reaction chambers of the integrated device.

Reaction chamber 1-108, a portion of the excitation source-to-chamber optics, and the reaction chamber-to-photodetector optics are located on integrated device 1-102. Excitation source 1-116 and a portion of the source-to-chamber components are located in instrument 1-104. In some embodiments, a single component may play a role in both coupling excitation light to reaction chamber 1-108 and delivering emission light from reaction chamber 1-108 to photodetector 1-110. Examples of suitable components, for coupling excitation light to a reaction chamber and/or directing emission light to a photodetector, to include in an integrated device are described in U.S. Pat. application Ser. No. 14/821,688, filed Aug. 7, 2015, titled “INTEGRATED DEVICE FOR PROBING, DETECTING AND ANALYZING MOLECULES” under Attorney Docket Number R0708.70004US02 and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES” under Attorney Docket Number R0708.70005US00, both of which are incorporated by reference in their entirety.

Pixel 1-112 is associated with its own individual reaction chamber 1-108 and at least one photodetector 1-110. The plurality of pixels of integrated device 1-102 may be arranged to have any suitable shape, size, and/or dimensions. Integrated device 1-102 may have any suitable number of pixels. The number of pixels in integrated device 1-102 may be in the range of approximately 100,000 pixels to 64,000,000 pixels or any value or range of values within that range. In some embodiments, the pixels may be arranged in an array of 1024 pixels by 2048 pixels. Integrated device 1-102 may interface with instrument 1-104 in any suitable manner. In some embodiments, instrument 1-104 may have an interface that detachably couples to integrated device 1-102 such that a user may attach integrated device 1-102 to instrument 1-104 for use of integrated device 1-102 to analyze at least one sample of interest in a suspension and remove integrated device 1-102 from instrument 1-104 to allow for another integrated device to be attached. The interface of instrument 1-104 may position integrated device 1-102 to couple with circuitry of instrument 1-104 to allow for readout signals from one or more photodetectors to be transmitted to instrument 1-104. Integrated device 1-102 and instrument 1-104 may include multi-channel, high-speed communication links for handling data associated with large pixel arrays (e.g., more than 10,000 pixels).

Instrument 1-104 may include a user interface for controlling operation of instrument 1-104 and/or integrated device 1-102. The user interface may be configured to allow a user to input information into the instrument, such as commands and/or settings used to control the functioning of the instrument. In some embodiments, the user interface may include buttons, switches, dials, and a microphone for voice commands. The user interface may allow a user to receive feedback on the performance of the instrument and/or integrated device, such as proper alignment and/or information obtained by readout signals from the photodetectors on the integrated device. In some embodiments, the user interface may provide feedback using a speaker to provide audible feedback. In some embodiments, the user interface may include indicator lights and/or a display screen for providing visual feedback to a user.

In some embodiments, instrument 1-104 may include a computer interface configured to connect with a computing device. Computer interface may be a USB interface, a FireWire interface, or any other suitable computer interface. Computing device may be any general purpose computer, such as a laptop or desktop computer. In some embodiments, computing device may be a server (e.g., cloud-based server) accessible over a wireless network via a suitable computer interface. The computer interface may facilitate communication of information between instrument 1-104 and the computing device. Input information for controlling and/or configuring the instrument 1-104 may be provided to the computing device and transmitted to instrument 1-104 via the computer interface. Output information generated by instrument 5-104 may be received by the computing device via the computer interface. Output information may include feedback about performance of instrument 1-104, performance of integrated device 1-102, and/or data generated from the readout signals of photodetector 1-110.

In some embodiments, instrument 1-104 may include a processing device configured to analyze data received from one or more photodetectors of integrated device 1-102 and/or transmit control signals to excitation source(s) 1-116. In some embodiments, the processing device may comprise a general purpose processor, a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof.) In some embodiments, the processing of data from one or more photodetectors may be performed by both a processing device of instrument 1-104 and an external computing device. In other embodiments, an external computing device may be omitted and processing of data from one or more photodetectors may be performed solely by a processing device of integrated device 1-102.

A cross-sectional schematic of integrated device 1-102 illustrating a row of pixels 1-112 is shown in FIG. 1B. Integrated device 1-102 may include coupling region 1-201, routing region 1-202, and pixel region 1-203. Pixel region 1-203 may include a plurality of pixels 1-112 having reaction chambers 1-108 positioned on a surface at a location separate from coupling region 1-201, which is where excitation light (shown as the dashed arrow) couples to integrated device 1-102. Reaction chambers 1-108 may be formed through metal layer(s) 1-106. One pixel 1-112, illustrated by the dotted rectangle, is a region of integrated device 1-102 that includes a reaction chamber 1-108 and a photodetection region having one or more photodetectors 1-110. In the illustrated embodiment, the pixel comprises a single reaction chamber 1-108. In some embodiments, each pixel may comprise two or more reaction chambers.

FIG. 1B illustrates the path of excitation (shown in dashed lines) by coupling a beam of excitation light to coupling region 1-201 and to reaction chambers 1-108. The row of reaction chambers 1-108 shown in FIG. 1B may be positioned to optically couple with waveguide 1-220. Excitation light may illuminate a sample located within a reaction chamber. The sample or reaction component (e.g., a fluorescent label) may reach an excited state in response to being illuminated by the excitation light. When a sample or reaction component is in an excited state, the sample or reaction component may emit emission light, which may be detected by one or more photodetectors associated with the reaction chamber. FIG. 1B schematically illustrates an optical axis of emission light (shown as the solid line) from a reaction chamber 1-108 to photodetector(s) 1-110 of pixel 1-112. The photodetector(s) 1-110 of pixel 1-112 may be configured and positioned to detect emission light from reaction chamber 1-108. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS” under Attorney Docket Number R0708.70002US02, which is incorporated by reference in its entirety. For an individual pixel 1-112, a reaction chamber 1-108 and its respective photodetector(s) 1-110 may be aligned along a common axis (along the y-direction shown in FIG. 1A). In this manner, the photodetector(s) may overlap with the reaction chamber within a pixel 1-112.

The directionality of the emission light from a reaction chamber 1-108 may depend on the positioning of the sample in the reaction chamber 1-108 relative to metal layer(s) 1-106 because metal layer(s) 1-106 may act to reflect emission light. In this manner, a distance between metal layer(s) 1-106 and a fluorescent marker positioned in a reaction chamber 1-108 may impact the efficiency of photodetector(s) 1-110, that are in the same pixel as the reaction chamber, to detect the light emitted by the fluorescent marker. The distance between metal layer(s) 1-106 and the bottom surface of a reaction chamber 1-108, which is proximate to where a sample may be positioned during operation, may be in the range of 100 nm to 500 nm, or any value or range of values in that range. In some embodiments the distance between metal layer(s) 1-106 and the bottom surface of a reaction chamber 1-108 is approximately 300 nm.

The distance between the sample and the photodetector(s) may also impact efficiency in detecting emission light. By decreasing the distance light has to travel between the sample and the photodetector(s), detection efficiency of emission light may be improved. In addition, smaller distances between the sample and the photodetector(s) may allow for pixels that occupy a smaller area footprint of the integrated device, which can allow for a higher number of pixels to be included in the integrated device. The distance between the bottom surface of a reaction chamber 1-108 and photodetector(s) may be in the range of 1 μm to 15 μm, or any value or range of values in that range. It should be appreciated that, in some embodiments, emission light may be provided through other means than an excitation light source and a reaction chamber. Accordingly, some embodiments may not include reaction chamber 1-108.

Photonic structure(s) 1-230 may be positioned between reaction chambers 1-108 and photodetectors 1-110 and configured to reduce or prevent excitation light from reaching photodetectors 1-110, which may otherwise contribute to signal noise in detecting emission light. As shown in FIG. 1B, the one or more photonic structures 1-230 may be positioned between waveguide 1-220 and photodetectors 1-110. Photonic structure(s) 1-230 may include one or more optical rejection photonic structures including a spectral filter, a polarization filter, and a spatial filter. Photonic structure(s) 1-230 may be positioned to align with individual reaction chambers 1-108 and their respective photodetector(s) 1-110 along a common axis. Metal layers 1-240, which may act as a circuitry for integrated device 1-102, may also act as a spatial filter, or polarization filter, in accordance with some embodiments. In such embodiments, one or more metal layers 1-240 may be positioned to block some or all excitation light from reaching photodetector(s) 1-110.

Coupling region 1-201 may include one or more optical components configured to couple excitation light from an external excitation source, for example, excitation source(s) 1-116 illustrated in FIG. 1A. Coupling region 1-201 may include grating coupler 1-216 positioned to receive some or all of a beam of excitation light. Examples of suitable grating couplers are described in U.S. patent application Ser. No. 15/844,403, filed Dec. 15, 2017, titled “OPTICAL COUPLER AND WAVEGUIDE SYSTEM” under Attorney Docket Number R0708.70021US01, and U.S. patent application Ser. No. 16/861,399, filed Apr. 29, 2020, titled “SLICED GRATING COUPLER WITH INCREASED BEAM ALIGNMENT SENSITIVITY” under Attorney Docket Number R0708.70071US01, each of which are hereby incorporated by reference herein in their entireties. Grating coupler 1-216 may couple excitation light to waveguide 1-220, which may be configured to propagate excitation light to the proximity of one or more reaction chambers 1-108. Alternatively, coupling region 1-201 may comprise other well-known structures for coupling light into a waveguide.

Components located off of the integrated device may be used to position and align the excitation source 1-116 to the integrated device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM” under Attorney Docket Number R0708.70010US02, which is incorporated by reference in its entirety. Another example of a beam-steering module is described in U.S. patent application Ser. No. 15/842,720, filed Dec. 14, 2017, titled “COMPACT BEAM SHAPING AND STEERING ASSEMBLY” under Attorney Docket Number R0708.70024US01, which is incorporated herein by reference.

A sample to be analyzed may be introduced into reaction chamber 1-108 of pixel 1-112. The sample may be a biological sample or any other suitable sample, such as a chemical sample. The sample may include multiple molecules and the reaction chamber may be configured to isolate a single molecule. In some instances, the dimensions of the reaction chamber may act to confine a single molecule within the reaction chamber, allowing measurements to be performed on the single molecule. Excitation light may be delivered into the reaction chamber 1-108, so as to excite the sample or at least one fluorescent marker attached to the sample or otherwise associated with the sample while it is within an illumination area within the reaction chamber 1-108.

In operation, parallel analyses of samples within the reaction chambers are carried out by exciting some or all of the samples within the wells using excitation light and detecting signals from sample emission with the photodetectors. Emission light from a sample may be detected by a corresponding photodetector and converted to at least one electrical signal. Information regarding various characteristics of the emission light (e.g., wavelength, luminescent lifetime, intensity, pulse duration and/or any other suitable characteristic) may be collected and used for subsequent analysis, as described herein. The electrical signals may be transmitted along conducting lines (e.g., metal layers 1-240) in the circuitry of the integrated device, which may be connected to an instrument interfaced with the integrated device. The electrical signals may be subsequently processed and/or analyzed. Processing or analyzing of electrical signals may occur on a suitable computing device either located on or off the instrument.

FIG. 1C illustrates a cross-sectional view of a pixel 1-112 of integrated device 1-102, according to some embodiments. FIG. 1D shows a circuit diagram of pixel 1-112. FIG. 1E shows an exemplary array of pixels 1-112 and processing circuit 1-114, which may be included in integrated device 1-102, according to some embodiments.

In FIGS. 1C and 1D, pixel 1-112 includes a photodetection region, which may be a pinned photodiode (PPD), two charge storage regions, which may be storage diodes (SD0 and SD1), and a readout region, which may be a floating diffusion (FD) region. Also as shown, pixel 1-112 also includes drain region D and transfer gates ST0, TX0, TX1, and REJ.

In some embodiments, photodetection region PPD, charge storage regions SD0 and SD1 and readout region FD may be formed on an integrated circuit substrate by doping parts of the substrate. For example, the substrate may be lightly doped and photodetection region PPD, charge storage regions SD0 and SD1, and readout region FD may be more heavily doped. In this example, the substrate may be lightly p-type doped and photodetection region PPD, charge storage regions SD0 and SD1, and readout region FD may be n-type doped. Alternatively, the substrate may be lightly n-type doped and photodetection region PPD, charge storage regions SD0 and SD1, and readout region FD may be p-type doped, as embodiments described herein are not so limited.

In some embodiments photodetection region PPD may be configured to generate charge carriers (e.g., photo-electrons) when incident photons are received therein. In some embodiments, charge storage regions SD0 and SD1 may be electrically coupled to photodetection region PPD and/or to one another. For example, pixel 1-112 may include one or more transfer channels electrically coupling charge storage regions SD0 and SD1 to photodetection region PPD and/or to one another. In some embodiments, the transfer channels may be formed by doping portions of the integrated circuit substrate disposed between the regions. For example, the portions may be doped with a same conductivity type as the regions (e.g., an n-type doped channel disposed between an n-type doped PPD and SD0). Referring to FIG. 1D, for example, a channel of a transistor coupled between photodetection region PPD and charge storage region SD0 is a transfer channel electrically coupling photodetection region PPD to charge storage region SD0. Similarly, a channel of a transistor coupled between charge storage regions SD0 and SD1 is a transfer channel electrically coupling charge storage region SD0 to SD1, a channel of a transistor coupled between charge storage region SD1 and readout region FD is a transfer channel electrically coupling charge storage region SD1 to readout region FD. A channel of a transistor coupled between photodetection region PPD and drain region D is a transfer channel between photodetection region PPD and drain region D.

In some embodiments, transfer gates ST0, TX0, TX1, and REJ may be configured to control the transfer of charge carriers from photodetection region PPD to storage regions SD0 and SD1, between charge storage regions SD0 and SD1, and/or between charge storage regions SD0 and SD1 and readout region FD. For example, transfer gates ST0, TX0, TX1, and REJ may be electrically coupled to and configured to bias the transfer channels electrically coupling the regions of pixel 1-112 to transfer the charge carriers between the regions when appropriate control signals are applied to the transfer gates. The transfer gates may me conductively (e.g., physically) coupled to the transfer channels, and/or may be positioned close enough to the transfer channels and/or separated by a thin enough insulator to capacitively couple to the transfer channels, according to various embodiments. In some embodiments, transfer gates described herein may be formed using a conductive material such as metal. Alternatively or additionally, in some embodiments, transfer gates described herein may be formed using a semiconductor material such as polysilicon. In some embodiments, materials used to form transfer gates described herein may be at least partially opaque.

In some embodiments, when a control signal is received at a transfer gate, the transfer gate may electrically couple the control signal to the transfer channel and bias the transfer channel, thereby increasing the conductivity of the transfer channel. In some embodiments, the transfer channel may be doped with a same conductivity type but a lower dopant concentration than the regions of pixel 1-112 electrically coupled by the transfer channel, thereby generating an intrinsic electric potential barrier between the regions. The intrinsic electric potential barrier may exist between the regions even when no external electric field is applied to the transfer gate or transfer channel. For example, the dopant concentration of the transfer channel between photodetection region PPD and charge storage region SD0 may generate an intrinsic electric potential barrier between photodetection region PPD and charge storage region SD0. In some embodiments, a control signal may be applied to the transfer gate, the control signal being configured to lower the intrinsic electric potential barrier between the regions electrically coupled by the transfer channel, thereby increasing the conductivity of the transfer channel, and causing a transfer of charge carriers between the regions. For example, for an n-type doped transfer channel, the control signal may have a voltage that is greater than a voltage at one of the regions (e.g., at the source terminal of the transfer channel) by at least a threshold voltage of the transfer channel, the threshold voltage being dependent on the size of the transfer channel, a substrate voltage of the integrated device 1-102 proximate the transfer channel, and other such parameters. Similarly, for a p-type doped transfer channel, the control signal may have a voltage that is lower than the voltage at the one of the regions by at least the threshold voltage. In some embodiments, a control circuit of integrated device 1-102 may be configured to generate and provide such control signals to the transfer gates, as described further herein.

In FIG. 1C, pixel 1-112 is shown in a configuration configured to receive incident photons in a direction in which photodetection region PPD, charge storage regions SD0 and SD1, and readout region FD are spaced from transfer gates REJ, ST0, TX0, and TX1 (e.g., front-side illumination). It should be appreciated, however, that in some embodiments, photodetection region PPD may be configured to receive incident photons in a direction in which transfer gates REJ, ST0, TX0, and TX1 are spaced from photodetection region PPD, charge storage regions SD0 and SD1, and readout region FD (e.g., back-side illumination). In some embodiments, such a configuration may improve the electrical characteristics of the transfer gates because the optical characteristics of the transfer gates have a reduced impact on the incident photons.

In FIG. 1D, pixel 1-112 further includes a reset (RST) transfer gate coupled to readout region FD and configured for coupling to a high voltage VDDP, and a row select (RS) transfer gate coupled between readout region FD and a bitline. When the integrated device 1-102 is coupled to a power source (e.g., at least a DC power supply), transfer gate RST may be coupled to high voltage VDDP, which is supplied by the power source and/or regulated by a voltage regulator of integrated device 1-102.

In some embodiments, transfer gate RST may be configured to reset a voltage of readout region FD. For example, when a reset signal is applied to transfer gate RST, transfer gate RST may bias the transfer channel electrically coupling readout region FD to high voltage VDDP, thereby increasing the conductivity of the transfer channel and transferring charge carriers from readout region FD to high voltage VDDP. In some embodiments, reset transfer gate RST may be further configured to reset the voltage of charge storage region SD0 and/or SD1. For example, when a reset signal is applied to reset transfer gate RST and a control signal is applied to transfer gate TX1, transfer gate TX1 may transfer charge carriers in charge storage region SD1 to readout region FD and transfer gate RST may transfer the charge carriers to high voltage VDDP. Similarly, when a reset signal is applied to reset transfer gate RST and control signals are applied to transfer gates TX1 and TX0, transfer gate TX0 may transfer charge carriers in charge storage region SD0 to SD1, transfer gate TX1 may transfer the charge carriers in charge storage region SD1 to readout region FD, and transfer gate RST may transfer the charge carriers to high voltage VDDP. In some embodiments, integrated device 1-102 may be configured to reset readout region FD and charge storage regions SD0 and SD1 before collecting and reading out charge carriers. For example, integrated device 1-102 may be configured to reset readout region FD, then reset charge storage region SD1, and then reset charge storage region SD0, before collecting and reading out charge carriers.

In some embodiments, the bitline may be coupled to processing circuitry on the integrated device 1-102 and/or an external circuit configured to receive a voltage level indicative of charge carriers read out to readout region FD. In some embodiments, processing circuitry 1-114 may include an analog-to-digital converter (ADC). In some embodiments, integrated device 1-102 may be configured to reset the voltage of readout region FD of each pixel before reading out charge carriers. For example, integrated device 1-102 may be configured to reset the voltage of readout region FD, sample the voltage, transfer charge carriers into readout region FD, and sample the voltage again. In this example, the second sampled voltage may be indicative of a number of the charge carriers transferred into readout region FD when compared to the first sampled voltage. In some embodiments, integrated device 1-102 may be configured to read out charge carriers from each pixel 1-112 to the bitline sequentially, such as row by row and/or column by column. It should be appreciated that some arrays of pixels 1-112 may have multiple bitlines electrically coupled to different ones and/or groups of pixels 1-112. In some embodiments, pixels of multiple columns may be read out to respective processing circuitry at the same time. For example, a first pixel of each column (e.g., pixels (1,1) and (1,2) and so on) may be read out to the respective processing circuitry at the same time, and then a second pixel of each column (e.g., pixels (2,1) and (2,2,) and so on) may be read out to the respective processing circuitry at the same time. It should be appreciated that, in some embodiments, processing circuitry may be provided for each row of the array as an alternative or in addition to each column. In some embodiments, integrated device 1-102 may include multiple units of processing circuitry, such as each being electrically coupled to a bitline.

It should be appreciated that, in accordance with various embodiments, transfer gates described herein may include semiconductor material(s) and/or metal, and may include a gate of a field effect transistor (FET), a base of a bipolar junction transistor (BJT), and/or the like. It should also be appreciated that control signals described herein applied to the various transfer gates may vary in shape and/or voltage, such as depending on the electric potential of the semiconductor region and of the regions electrically coupled to the semiconductor region (e.g., neighboring regions).

In some embodiments, pixels described herein may include more than two charge storage regions. For example, pixel 2-112 described herein in connection with FIGS. 1G-1I includes three charge storage regions.

FIG. 1E is a plan view of alternative pixel 1-112′, according to some embodiments. In some embodiments, pixel 1-112′ may be configured in the manner described for pixel 1-112. In FIG. 1E, drain region D of pixel 1-112′ is positioned on a same side of photodetection region PPD as charge storage regions SD0 and SD1 and readout region FD. Also shown in FIG. 1E, photodetection region PPD may include a mask with a triangular opening, with a base of the triangular opening on a side of photodetection region proximate charge storage regions SD0 and SD1 and drain region D, and a corresponding apex of the triangular opening on a side of photodetection region PPD opposite drain region D and charge storage regions SD0 and SD1.

In some embodiments, photodetection region PPD may be configured to induce an intrinsic electric field in a direction from photodetection region PPD toward charge storage regions SD0 and SD1 and drain region D. For example, photodetection region PPD may be formed by doping a substrate of integrated device 1-102 through the opening, resulting in a higher dopant concentration in the region of the substrate exposed through the opening than in the region covered by the mask during doping. In this example, the larger quantity of dopants (e.g., n-type dopants) at the base end of the triangular opening may cause the electric potential at the base end of photodetection region PPD proximate drain region D and charge storage region SD0 to be lower than the electric potential at the apex end of photodetection region PPD on the opposite side of photodetection region PPD. The intrinsic electric field in photodetection region PPD may be present even in the absence of an external electric field being applied to pixel 1-112. The inventors recognized that the intrinsic electric field of photodetection region PPD increases the rate of charge transfer from photodetection region PPD to drain region D and/or storage regions SD0 and SD1, increasing the efficiency with which charge carriers are drained and/or collected during operation of pixel 1-112. In the example of FIG. 1E, the intrinsic electric field may be directed along the dotted arrow between drain region and charge storage region SD0. For example, the intrinsic electric field may cause charge carriers to flow along the dotted arrow, and an extrinsic electric field induced by a control signal being applied to transfer gate REJ or ST0 may cause the charge carriers to flow to drain region D or charge storage region SD0, respectively.

FIG. IF is a top schematic view of the pixel 1-112′, according to some embodiments. As shown in FIG. 1F, contacts may be disposed over portions of pixel 1-112′. In some embodiments, the contacts may be configured to block incident photons from reaching portions of pixel 1-112′ other than photodetection region PPD and/or from reaching photodetection regions of neighboring pixels at oblique angles of incidence. For example, the contacts may be elongated in a direction parallel to the optical axis along which photodetection PPD is configured to receive incident photons. In some embodiments, the contacts may be formed using an opaque material such as tungsten. The inventors have recognized that contacts described herein prevent many or all incident photons from reaching charge storage regions SD0 and SD1 along optical paths other than the optical axis, thereby preventing the incident photons from generating noise charge carriers in charge storage regions SD0 and SD1.

In FIG. 1F, a pair of contacts is disposed on opposite sides of photodetection region PPD, with a first contact of the pair disposed closer to the apex of the triangular opening of the mask and a second contact of the pair disposed closer to the base of the triangular opening of the mask. The second contact may be configured to block incident photons from reaching charge storage regions SD0 and SD1. A third contact is disposed at an end of pixel 1-112 opposite the end at which photodetection region PPD is disposed. The first and third contacts are disposed between the pixel 1-112 and respective neighboring pixels, and the second contact is positioned between photodetection region PPD and transfer gates ST0 and REJ. It should be appreciated that, in some embodiments, the pair of contacts on opposite sides of photodetection region PPD may be replaced with at least one contact wall that at least partially surrounds photodetection region PPD, such as a single cylindrical contact wall.

FIG. 1G is a cross-sectional view of an alternative example pixel 2-112, which may be included in integrated device 1-102, according to some embodiments. In some embodiments, pixel 2-112 may be configured in the manner described for pixel 1-112 in connection with FIGS. 1A-1F. For example, as shown in FIG. 1G, region FD, and drain region D, and transfer gates, each of which may be configured in the manner described for pixel 1-112. Pixel 2-112 further includes charge storage region SD2 electrically coupled between charge storage region SD1 and readout region FD. For example, transfer channels may electrically couple charge storage region SD1 to charge storage region SD2 and charge storage region SD2 to readout region FD. In FIG. 1G, transfer gate TX0 is configured to control a transfer of charge carriers from charge storage region SD1 to charge storage region SD2, and transfer gate TX1 is configured to control a transfer of charge carriers from charge storage region SD2 to readout region FD.

FIG. 1H is a circuit diagram of pixel 2-112, according to some embodiments. As shown in FIG. 1H, the transfer channel electrically coupling charge storage region SD1 to charge storage region SD2 is a channel of a transistor having transfer gate TX0 and the transfer channel electrically coupling charge storage region SD2 to readout region FD is a channel of a transistor having transfer gate TX1. The other transistors of pixel 2-112 shown in FIG. 1H, such as the transistor having reset gate RST and the transistor having row select transfer gate RS may be configured in the manner described for pixel 1-112 FIGS. For example, an array of pixels 2-112 may be arranged in a configuration with processing circuitry as described herein for pixel 1-112 FIGS.

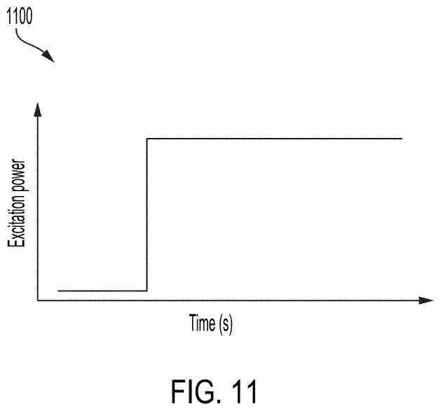

FIG. 11 is a top view of pixel 2-112′, which may be included in integrated device 1-102 of FIG. 1B, according to some embodiments. In some embodiments, pixel 2-112′ may be configured in the manner described herein for pixel 1-112′. For example, photodetection region PPD of pixel 2-112′ may be configured to induce an intrinsic electric field in the direction from photodetection region PPD toward charge storage region SD0 and drain region D.

In some embodiments, the integrated device comprises a sample preparation module capable of performing processes for preparing a biological sample, e.g., for detection and/or analysis. In some embodiments, a process for preparing a biological sample described herein may be used to identify properties or characteristics of a sample, including the identity or sequence (e.g., nucleotide sequence or amino acid sequence) of one or more target molecules in a biological sample. In some embodiments, the biological sample is a single cell, mammalian cell tissue, animal sample, fungal sample, or plant sample. In some embodiments, the biological sample is a blood sample, saliva sample, sputum sample, fecal sample, urine sample, buccal swab sample, amniotic sample, seminal sample, synovial sample, spinal sample, or pleural fluid sample. In some embodiments, a biological sample is from a human, a non-human primate, a rodent, a dog, a cat, a horse, or any other mammal. In some embodiments, a biological sample is from a bacterial cell culture (e.g., an E. coli bacterial cell culture). In some embodiments, the one or more target molecules are nucleic acids. In some embodiments, the one or more target molecules are proteins.

In some embodiments, a process for preparing a biological sample may include one or more sample transformation steps, such as sample lysis, sample purification, sample fragmentation, purification of a fragmented sample, library preparation (e.g., nucleic acid library preparation), purification of a library preparation, sample enrichment (e.g., using affinity SCODA), and/or detection/analysis of a target molecule.

In some embodiments, a sample (e.g., a sample comprising cells or tissue), may be prepared, e.g., lysed (e.g., disrupted, degraded and/or otherwise digested). In some embodiments, a sample comprising cells or tissue is lysed using any one of known physical or chemical methodologies to release a target molecule (e.g., a target nucleic acid or a target protein) from said cells or tissues. In some embodiments, a sample may be lysed using any method known to a person of ordinary skill in the art. For example, a sample may be lysed using electrolytic method, an enzymatic method, a detergent-based method, and/or mechanical homogenization. In some embodiments, a sample (e.g., complex tissues, gram positive or gram-negative bacteria) may require multiple lysis methods performed in series. In some embodiments, a sample is prepared, e.g., lysed, in the presence of a buffer system. This buffer system may be used to make a slurry of the sample, to suspend the sample, and/or to stabilize the sample during any known lysis methodology, including those methods described herein. In some embodiments, methods that cause lysis by mechanical homogenization include, but are not limited to bead-beating, heating (e.g., to high temperatures sufficient to disrupt cell walls, e.g., greater than 50° C., 60° C., 70° C., 80° C., 90° C., or 95° C.), syringe/needle/microchannel passage (to cause shearing), sonication, or maceration with a grinder. In some embodiments, sample preparation comprises cell disruption (i.e., subsequent removal of unwanted cell and tissue elements following lysis). In some embodiments, cell disruption involves protein and/or nucleic acid precipitation. In some embodiments, following precipitation, the lysed and disrupted sample is subjected to centrifugation. In some embodiments, following centrifugation, the supernatant is discarded. In some embodiments, proteins or peptides are immunoprecipitated.

In some embodiments, a sample (e.g., a sample comprising a target nucleic acid or a target protein) may be purified, e.g., following lysis, in a process in accordance with the instant disclosure. In some embodiments, a sample may be purified using chromatography (e.g., affinity chromatography that selectively binds the sample) or electrophoresis. In some embodiments, a sample may be purified in the presence of precipitating agents. In some embodiments, after a purification step or method, a sample may be washed and/or released from a purification matrix (e.g., affinity chromatography matrix) using an elution buffer. In some embodiments, a purification step or method may comprise the use of a reversibly switchable polymer, such as an electroactive polymer. In some embodiments, a sample may be purified by electrophoretic passage of a sample through a porous matrix (e.g., cellulose acetate, agarose, acrylamide).

In some embodiments, a sample (e.g., a sample comprising a target nucleic acid or a target protein) may be fragmented (i.e., digested) in a process in accordance with the instant disclosure. In some embodiments, a nucleic acid sample may be fragmented to produce small (<1 kilobase) fragments for sequence specific identification to large (up to 10+ kilobases) fragments for long read sequencing applications. Fragmentation of nucleic acids or proteins may, in some embodiments, be accomplished using mechanical (e.g., fluidic shearing), chemical (e.g., iron (Fe+) cleavage) and/or enzymatic (e.g., restriction enzymes, tagmentation using transposases) methods. In some embodiments, a protein sample may be fragmented to produce peptide fragments of any length. Fragmentation of proteins may, in some embodiments, be accomplished using chemical and/or enzymatic (e.g., proteolytic enzymes such as trypsin) methods. In some embodiments, mean fragment length may be controlled by reaction time, temperature, and concentration of sample and/or enzymes (e.g., restriction enzymes, transposases). In some embodiments, a nucleic acid may be fragmented by tagmentation such that the nucleic acid is simultaneously fragmented and labeled with a fluorescent molecule (e.g., a fluorophore). In some embodiments, a fragmented sample may be subjected to a round of purification (e.g., chromatography or electrophoresis) to remove small and/or undesired fragments as well as residual payload, chemicals and/or enzymes (e.g., transposases) used during the fragmentation step.

In some embodiments, a sample (e.g., a sample comprising a target nucleic acid or a target protein) may be enriched for a target molecule in a process in accordance with the instant disclosure. Enrichment is typically used when the complexity of the un-enriched sample exceeds the capacity of the sequencing platform, or when the target molecule is present in the sample at a low abundance (e.g., such that it cannot be easily detected by the sequencing platform). Enrichment involves the use of a mechanism that selectively amplifies the target molecule. This enrichment may involve the use of antibodies, aptamers, size-based selection, or electrostatic charge-based selection in order to selectively amplify the target molecule(s) (e.g., target protein(s) or target nucleic acid(s)). Enrichment may typically be used when the intent of the sample preparation is to sequence specific target molecules. Enrichment may be used to perform or conduct a proteomic, genomic, or metagenomic analysis or survey, when the target molecules are related or homologous to one another. In some embodiments, a sample is enriched for a target molecule using an electrophoretic method. In some embodiments, a sample is enriched for a target molecule using affinity SCODA. In some embodiments, a sample is enriched for a target molecule using field inversion gel electrophoresis (FIGE). In some embodiments, a sample is enriched for a target molecule using pulsed field gel electrophoresis (PFGE). In some embodiments, the matrix used during enrichment (e.g., a porous media, electrophoretic polymer gel) comprises immobilized affinity agents (also known as ‘immobilized capture probes’) that bind to target molecule present in the sample. In some embodiments, a matrix used during enrichment comprises 1, 2, 3, 4, 5, or more unique immobilized capture probes, each of which binds to a unique target molecule and/or bind to the same target molecule with different binding affinities.

In addition to amplification of the target molecule, or as an alternative to amplification of the target molecule, a sample may be enriched (e.g., for a low abundance target molecule) by depletion of unwanted non-target molecules (e.g., high-abundance proteins (e.g. albumin)). Depletion of unwanted non-target molecules may be performed using similar capture strategies as discussed above. When using a depletion strategy, the capture probes will bind to unwanted, non-target molecules and allow for target molecules to remain in solution. This strategy equally enables enrichment of the target molecule (i.e., increased relative concentrations of the target molecule(s)).

In some embodiments, the target nucleic acid is enriched (e.g., enriched using electrophoretic methods, e.g., affinity SCODA) prior to determining the sequence of the target nucleic acid. In some embodiments, provided herein are methods of determining the sequences of a plurality of target nucleic acids (e.g., at least 2, 3, 4, 5, 10, 15, 20, 30, 50, or more) present in a sample (e.g., a purified sample, a cell lysate, a single-cell, a population of cells, or a tissue). In some embodiments, a sample is prepared as described herein (e.g., lysed, purified, fragmented, and/or enriched for a target nucleic acid) prior to determining the sequence of a target nucleic acid or a plurality of target nucleic acids present in a sample. In some embodiments, a target nucleic acid is an enriched target nucleic acid (e.g., enriched using electrophoretic methods, e.g., affinity SCODA).

In some embodiments, a target molecule or target molecules may be detected after enrichment and subsequent release to enable analysis of said target molecule(s) and its upstream sample, in a process in accordance with the instant disclosure. In some embodiments, a target nucleic acid may be detected using gene sequencing, absorbance, fluorescence, electrical conductivity, capacitance, surface plasmon resonance, hybrid capture, antibodies, direct labeling of the nucleic acid (e.g., end-labeling, labeled tagmentation payloads), non-specific labeling with intercalating dyes (e.g., ethidium bromide, SYBR dyes), or any other known methodology for nucleic acid detection. In some embodiments, a target protein or peptide fragment may be detected using absorbance, fluorescence, mass spectroscopy, amino acid sequencing, or any other known methodology for protein or peptide detection.

In some embodiments, a device further comprises a pump (e.g., a peristaltic pump) configured to transport one or more fluids into, within, or out of any one of modules of the device. In some embodiments, a device further comprises a pump (e.g., a peristaltic pump) configured to transport one or more fluids within, or through any of the microfluidic channels of cartridges received by the device or modules of the device. In some embodiments, a device is configured to transport fluids with a fluid flow resolution of less than or equal to 1000 microliters, less than or equal to 100 microliters, less than or equal to 50 microliters, or less than or equal to 10 microliters. In some embodiments, the device is configured to receive two or more cartridges at the same time. In some embodiments, the device is configured to establish fluidic communication between two or more cartridges received by the device at the same time. In some embodiments, the device is configured to receive two or more cartridges sequentially.

In some embodiments, the peristaltic pump comprises an apparatus comprising a roller and a fluidic device (e.g., a cartridge). In some embodiments, the peristaltic pump comprises an apparatus comprising a roller and a crank-and-rocker mechanism connected to the roller. In some embodiments, the device comprises a sample preparation module, the sample preparation module comprising a peristaltic pump comprising a fluidic cartridge comprising a base layer having a surface comprising channels, wherein at least a portion of at least some of the channels have a substantially triangularly-shaped cross-section having a single vertex at a base of the channel and having two other vertices at the surface of the base layer. A detection module may be downstream of the sample preparation module. In some embodiments, the sample preparation region comprises more than one fluidic cartridge.

According to some aspects, a fluidic system is provided. The fluidic system, according to some embodiments, can be used for transferring fluid to a fluidic receptacle, e.g., using peristaltic pumping. According to some embodiments, the fluidic system comprises a cartridge or module configured to receive a fluidic receptacle using alignment features. According to certain embodiments, the cartridge or module is configured to be fluidically connected to a fluidic receptacle via alignment features. This may allow cartridge or module of a device to be simultaneously aligned with and fluidically connected to the fluidic receptacle. Connections of this variety advantageously reduce a dead volume of a fluidic connection between the cartridge or module of the device and the fluidic receptacle, (e.g., by reducing the need for excess channel length), according to certain embodiments.

Two components are fluidically connected if, under some configurations of an embodiment, fluid may pass between them. For example, a first fluidic component and a second fluidic component may be in fluidic communication if they are connected by a channel, a microchannel, or a tube. As another example, two components separated by a valve would still be considered fluidically connected, as long as the valve could be configured to permit fluid flow between the two components. In contrast, two components that are only connected mechanically, without a fluidic pathway between them, would not be considered to be fluidically connected. Fluidically connected components may be directly fluidically connected (i.e., connected by a fluidic pathway that does not pass through any intervening components). However, fluidically connected components may in some cases be connected by a fluidic pathway through 1, 2, 3, 4, 5, 8, 10, 15, 20, or more intervening components.

The device, according to some embodiments, comprises a channel. The channel, in some embodiments, is fluidically connected to an alignment feature of the device. The alignment feature may be a first alignment feature of the device. According to some embodiments, the device further comprises a second alignment feature. The second alignment feature is connected to the channel, according to certain embodiments, e.g., via a fluidic connection established by a fluidic pathway passing through a fluidic receptacle. However, in some embodiments or configurations, the second alignment feature is not connected to the channel. According to some embodiments, the device is configured to receive a fluidic receptacle. For example, the device is configured to hold the fluidic receptacle, according to some embodiments. The device comprises a cartridge, according to some embodiments. In some embodiments, the device comprises a solid substrate (e.g., a solid substrate of cartridge). The solid substrate may comprise a recess configured to receive the fluidic receptacle.

The fluidic receptacle may have any of a variety of appropriate geometries. For example, the fluidic receptacle may have a substantially rectangular lateral profile, according to certain embodiments. In some embodiments, the fluidic receptacle has a narrow transverse dimension, when compared to a minimum lateral dimension of the fluidic receptacle. In some embodiments, the fluidic receptacle may be configured to house a substrate (e.g., an integrated device, a chip). According to some embodiments, the fluidic receptacle is a flow cell (e.g., a cell comprising an interior, an inlet, and an outlet, configured to receive a fluid through an inlet and transmit a fluid through an outlet). For example, the fluidic receptacle may be a flow cell comprising an integrated device. In some embodiments, the fluidic receptacle has an interior (e.g., an interior chamber). In some embodiments, the interior of the device is capable of being at least partially or completely filled with a fluid. The interior of the fluidic device has a volume, in some embodiments. In some embodiments, the fluidic receptacle has more than one interior. For example, the fluidic receptacle may comprise 1, 2, 3, 4, 5, or more interiors. According to some embodiments, at least some of these interiors are fluidically connected to one another. In some embodiments, these interiors are divided from one another, such that fluid cannot flow directly from one interior to another. The inclusion of multiple interiors of the fluidic receptacle is advantageous, according to some embodiments, because it can allow multiple integrated devices or integrated device portions to be filled simultaneously. The simultaneous filling of multiple integrated device or integrated device portions may allow greater uniformity of fluid flow, and/or may allow the analysis of multiple samples that are simultaneously allowed to interact with separate integrated devices or integrated device portions.

Each interior of the fluidic receptacle may comprise an inlet and an outlet. According to some embodiments, the inlet and/or the outlet can be connected to the fluidic device (e.g., via one or more alignment features). In some embodiments, fluid (e.g., a fluid sample comprising a biomolecules such as a peptide or polynucleotide) is flowed through one or more interiors of the fluidic receptacle. Flowing fluid through an interior can, in some embodiments, at least partially or completely fill the interior of the fluidic receptacle.

In some embodiments, a cartridge or module of a device comprises a base layer having a surface comprising channels. In some embodiments, at least a portion of at least some of the channels have a substantially triangularly-shaped cross-section having a single vertex at a base of the channel and having two other vertices at the surface of the base layer. In some embodiments, at least a portion of at least some of the channels have a surface layer. The surface layer may comprise an elastomer. The surface layer may be configured to substantially seal off a surface opening of the channel.

As used herein, the term “channel” will be known to those of ordinary skill in the art and may refer to a structure configured to contain and/or transport a fluid. A channel generally comprises: walls; a base (e.g., a base connected to the walls and/or formed from the walls); and a surface opening that may be open, covered, and/or sealed off at one or more portions of the channel. In some embodiments, a surface portion that is sealed off is completely sealed off. In some embodiments, a surface portion that is sealed off is substantially sealed off. A surface opening may be substantially sealed off if more than 50%, more than 60%, more than 75%, more than 90%, or more than 95% of the surface opening is sealed off. In some embodiments, a surface opening is sealed off by an elastomer.

As used herein, the term “microchannel” refers to a channel that comprises at least one dimension less than or equal to 1000 microns in size. For example, a microchannel may comprise at least one dimension (e.g., a width, a height) less than or equal to 1000 microns (e.g., less than or equal to 100 microns, less than or equal to 10 microns, less than or equal to 5 microns) in size. In some embodiments, a microchannel comprises at least one dimension greater than or equal to 1 micron (e.g., greater than or equal to 2 microns, greater than or equal to 10 microns). Combinations of the above-referenced ranges are also possible (e.g., greater than or equal to 1 micron and less than or equal to 1000 microns, greater than or equal to 10 micron and less than or equal to 100 microns). Other ranges are also possible. In some embodiments, a microchannel has a hydraulic diameter of less than or equal to 1000 microns. As used herein, the term “hydraulic diameter” (DH) will be known to those of ordinary skill in the art and may be determined as: DH=4A/P, wherein A is a cross-sectional area of the flow of fluid through the channel and P is a wetted perimeter of the cross-section (a perimeter of the cross-section of the channel contacted by the fluid).

In some embodiments, the fluidic components can operate to move liquids to, from, or between reservoirs and/or channels (e.g., an incubation channel) of a device. In some embodiments, the device components can operate to move liquids through channel(s) of a device, e.g., to, from, or between reservoirs and/or other channels (e.g., an incubation channel) of a fluidic device. In some embodiments, the device components move liquids via a peristaltic pumping mechanism (e.g., apparatus) that is configured to interact with an elastomeric component (e.g., surface layer comprising an elastomer) associated with a channel of a cartridge or module to pump fluid through the channel.

In some embodiments, fluidic exchange occurs during loading of a module or cartridge. For example, in some embodiments, fluidic exchange occurs during loading of a biological sample onto a sample preparation module of an integrated device. Fluidics exchange during loading a sample may involve addition or removal of solutions (e.g., buffered solutions or solutions for lysis of the sample) into a reservoir that contains the sample. In some embodiments, fluidics exchange during loading a sample may involve fluidic transfer of the sample from a first channel, microchannel or reservoir (e.g., a channel, microchannel or reservoir used for sample input) to a second channel, microchannel or reservoir (e.g., for lysis, fragmentation, digestion, enrichment, etc.). Further, in some embodiments, fluidic exchange occurs during loading of one or more target molecules into a detection module or device (e.g., a sequencing module or sequencing device as described herein). In some embodiments, fluidics exchange that occurs during loading of one or more target molecules into a detection module or device involves addition or removal of solutions involved in detection (e.g., a solution comprising a fluorophore or reference molecule).

In some embodiments, fluidic exchange occurs during detection of one or more target molecules (e.g., a target protein, polypeptide, or nucleic acid to be sequenced) or acquisition of data. For example, in some embodiments, fluidic exchange occurs during detection and/or data acquisition in a sample well (e.g., a sample well comprising one or more target molecules) or reaction chamber. In some embodiments, fluidic exchange occurring during detection and/or data acquisition involves addition or removal of a solution used for detection and/or data acquisition (e.g., a buffer comprising a fluorophore for detection). In some embodiments, fluidic exchange occuring during detection and/or data acquisition involves addition or removal of a solution used for stripping, reloading, and/or unloading a sample well or reaction chamber (a solution used for stripping coating molecules from a reaction chamber surface).

In some embodiments, devices, methods of using same, and associated fluidics, among other disclosure are as described in U.S. patent application Ser. No. 17/083,106, filed Oct. 28, 2020, entitled “PERISTALTIC PUMPING OF FLUIDS AND ASSOCIATED METHODS, SYSTEMS, AND DEVICES”; U.S. patent application Ser. No. 17/083,126, filed Oct. 28, 2020, entitled “PERISTALTIC PUMPING OF FLUIDS FOR BIOANALYTICAL APPLICATIONS AND ASSOCIATED METHODS, SYSTEMS, AND DEVICES;” U.S. patent application Ser. No. 17/082,223, filed Oct. 28, 2020, entitled “SYSTEMS AND METHODS FOR SAMPLE PREPARATION”; U.S. patent application Ser. No. 17/082,226, filed Oct. 28, 2020, entitled “METHODS AND DEVICES FOR SEQUENCING”; U.S. patent application Ser. No. 17/153,490, filed Jan. 20, 2021, entitled “COMPOUNDS AND METHODS FOR SELECTIVE C-TERMINAL LABELING;” and International Patent Application No. PCT/US2021/028471, filed Apr. 21, 2021, entitled “DEVICES AND METHODS FOR SEQUENCING;” the entire contents of each of which are incorporated herein by reference.

III. Techniques for Chip Reuse

As described herein, the inventors have developed techniques for reusing a sensor chip of an integrated device to process successive portions of a sample. The inventors have recognized that simply loading the integrated device with an additional sample aliquot, without more, leads to inaccurate results, as previously loaded sample may interfere with signal collection of newly loaded sample. Thus, the techniques described herein relate to methods for unloading previously loaded sample from reaction chambers of an integrated device and reusing the integrated device after a subsequent sample is loaded.

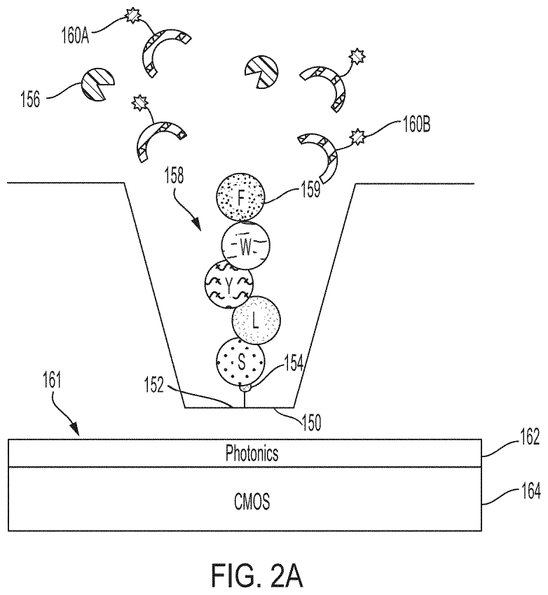

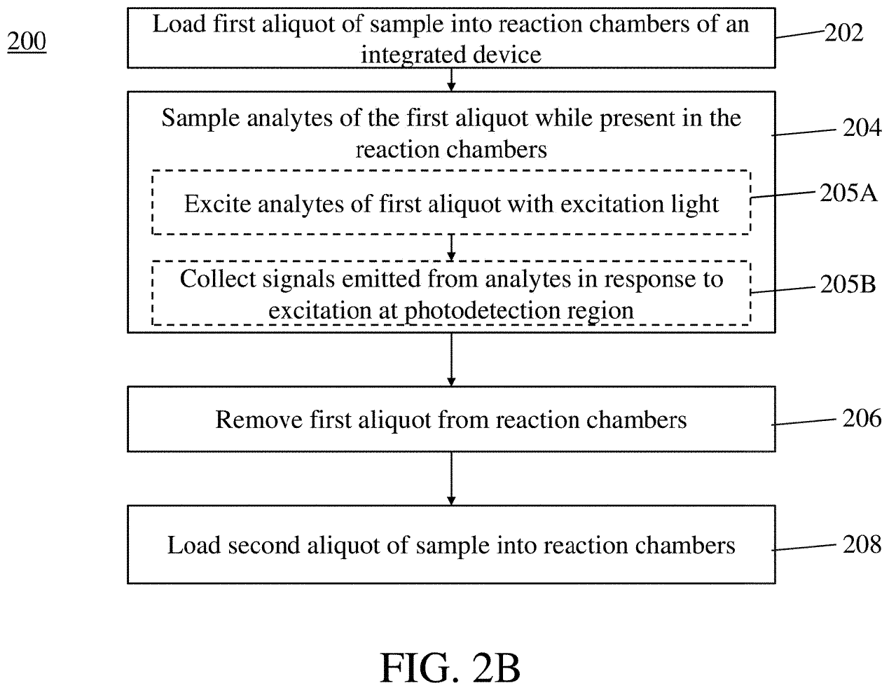

FIG. 2A is an example diagram of an analyte 159 having been immobilized in a chamber 158 of an integrated device, according to some embodiments. The analyte 159 may comprise a biomolecule, such as a polypeptide or polynucleotide, for example. FIG. 2A provides an example of a reaction chamber 158 with an analyte 159 (such as a chain of peptides) to be sampled. The analyte 159 may be bound to a surface 150 of the reaction chamber 158 via a bond between a coating molecule 152 coating the surface 150 of the reaction chamber 158 (e.g., biotin) and a coupling moiety 154 (e.g., streptavidin) bound to the analyte 159. The bond between the coating molecule 152 and the coupling moiety 154 immobilizes the analyte 149 in the reaction chamber 158 while sampling of the analyte 159 is performed. The analyte 159 may be labeled with a fluorescent dye. Labeling the analyte may be facilitated with a recognition molecule 160A, 160B configured to bind to a particular analyte. The reaction chamber 158 may be disposed proximate to a photodetection region 161 comprising a complimentary metal oxide semiconductor chip 164 and photonic components162 for collecting emission light from the reaction chamber 158. Subsequent to sampling, at least a portion of the analyte 159 may be cleaved by a cutter molecule 156, as described herein.

FIG. 2B is an example process for reusing an integrated device of FIG. 1A to process multiple samples, according to some embodiments. The process 200 begins at act 202, where a first aliquot of sample may be loaded into reaction chambers of an integrated device. For example, a sample comprising a large number of analyte species may be divided into multiple aliquots to be processed successively with the integrated device. A first aliquot may be loaded into reaction chambers of the integrated device. For example, surfaces of the reaction chambers may be coated with a coating molecule, such as biotin. Analytes of the first aliquot of sample may be bound to coupling moieties, such as streptavidin. When the first aliquot is loaded into the reaction chambers, the analytes are fixed (or affixed) to the surfaces of the reaction chambers by the bonding of the coupling moiety (of the sample) to the coating molecule (immobilized to the reaction chamber surface).

At act 204 of FIG. 2B, analytes of the first aliquot may be sampled while the analytes are present in the reaction chambers. Sampling an analyte may comprise delivering excitation light to the reaction chamber to excite the analyte and/or a fluorescent label attached thereto at act 205A. The excited sample emits signals (e.g., photons) which may be collected by a photodetection region of the integrated device at act 205B. The emitted signals may be used to identify the analyte, in some embodiments.

In some embodiments, the analyte comprises one peptide, or multiple peptides, to be sampled. In some embodiments, the analyte comprises one or more peptides, and the fluorescent label to be excited is conjugated to an amino acid recognition molecule. In some embodiments, peptide analyte identification can proceed by contacting the polypeptide with one or more amino acid recognition molecules that associate with one or more types of terminal amino acids. In some embodiments, cleaving molecules, or cutters, as shown in FIG. 2A may be used to remove a portion of the analyte (e.g., by removing a peptide from the chain of peptides) and a next portion of the analyte may be sampled.

Once sufficient signal from the analyte has been collected, the first aliquot may be removed from the reaction chambers of the integrated device at act 206. For example, it may be desired to load additional sample (i.e., additional aliquots of sample) into the integrated device in order to collect information regarding the additional sample. However, as described herein, previously processed analytes present in the reaction chambers may interfere with signal collection of newly loaded analytes. Thus, the first aliquot may be removed from the integrated device before loading additional sample into the integrated device.

Exemplary techniques for unloading the sensor chip include (a) disrupting the bonded anchoring between the coupling moiety and coating molecule (e.g., between streptavidin and biotin); (b) stripping the molecule (e.g., biotin) coating from the surface of the sensor chip; (c) enzymatically digesting the sample analyte in occupied reaction chambers; and (d) melting a bond between the sample analyte and the coupling moiety (e.g., streptavidin, biotin). Such techniques will be further described herein.

Subsequent to removing the first aliquot from the integrated device at act 206, a second aliquot of sample may be loading into the reaction chambers at act 208. Analytes of the second aliquot may likewise be bound to coupling moieties configured to bind to coating molecules coating (e.g., bound to) the surfaces of the reaction chambers. Analytes of the second aliquot may be sampled to collect information regarding the analytes.

The above-described process of loading and unloading an integrated device with multiple aliquots of a sample may be repeated as desired. The process allows for processing samples with a large number of analyte species by dividing the sample into multiple aliquots and processing the aliquots consecutively. The unloading techniques described herein prevent interference which may result from previously loaded analytes emitting signal during sampling of newly loaded aliquots. Any of the unloading techniques described below may be used in combination (e.g., in series) to accomplish the researcher's objectives.

Disrupting Bonds Between Coating Molecules and Coupling Moieties

A first exemplary technique for unloading a portion of a sample from reaction chambers of the integrated device involves disrupting covalent bonds from between the coating molecules coating surfaces of the reaction chambers and coupling moieties bound to analytes of the sample. Disrupting the bond between coating molecules and coupling moieties releases the fixed analyte from the surface of a reaction chamber, allowing the analyte to be flushed from the integrated device. In some embodiments, disrupting a covalent bond between a coating molecule and a coupling moiety comprises dissociating the coupling moiety and the coating molecule.

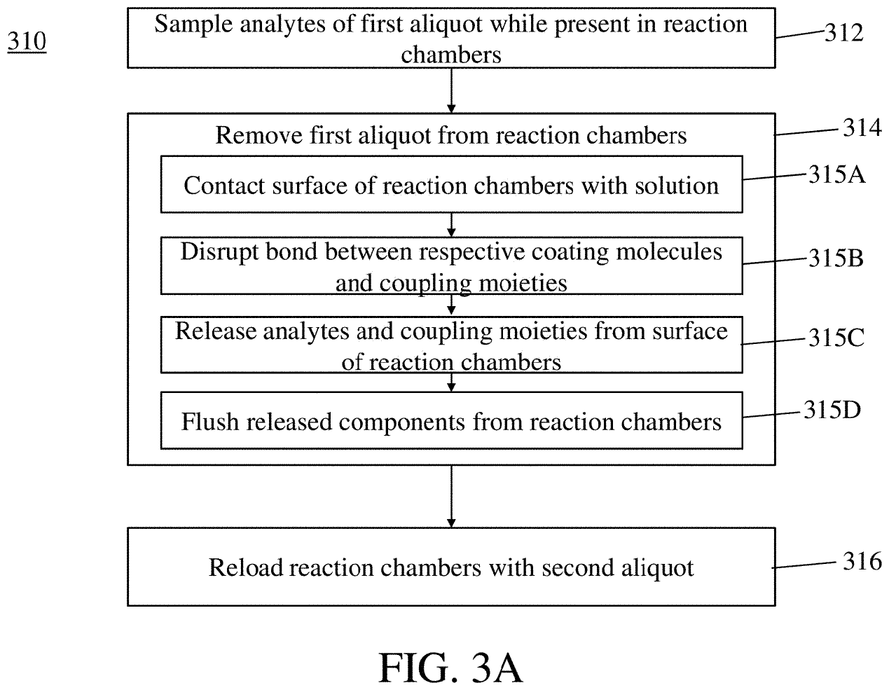

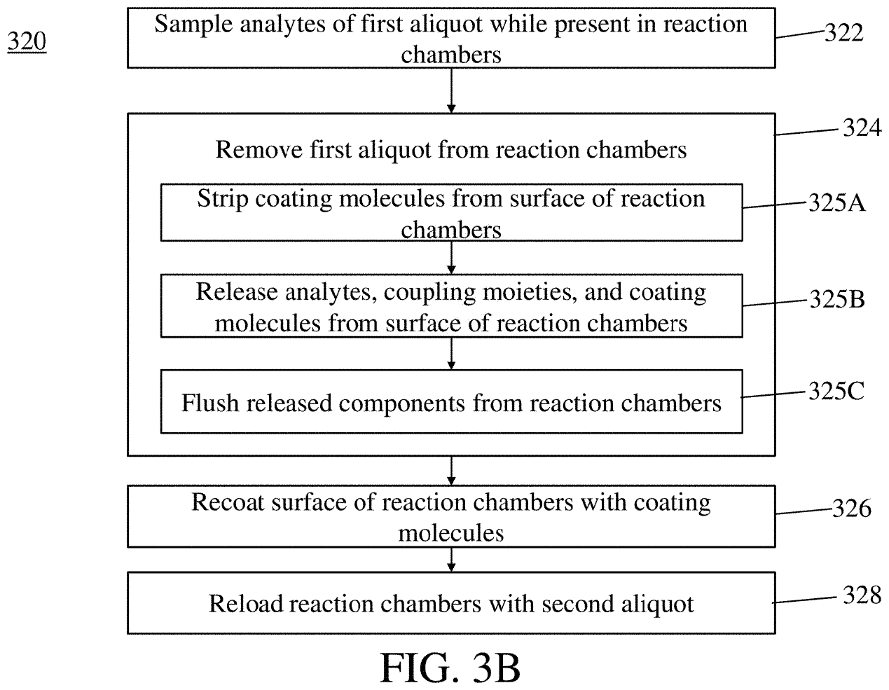

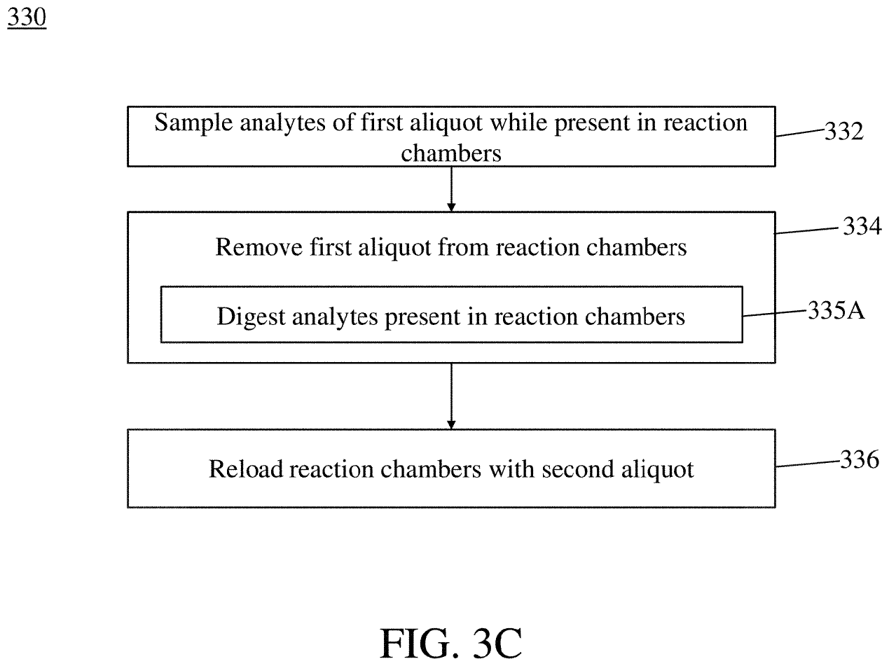

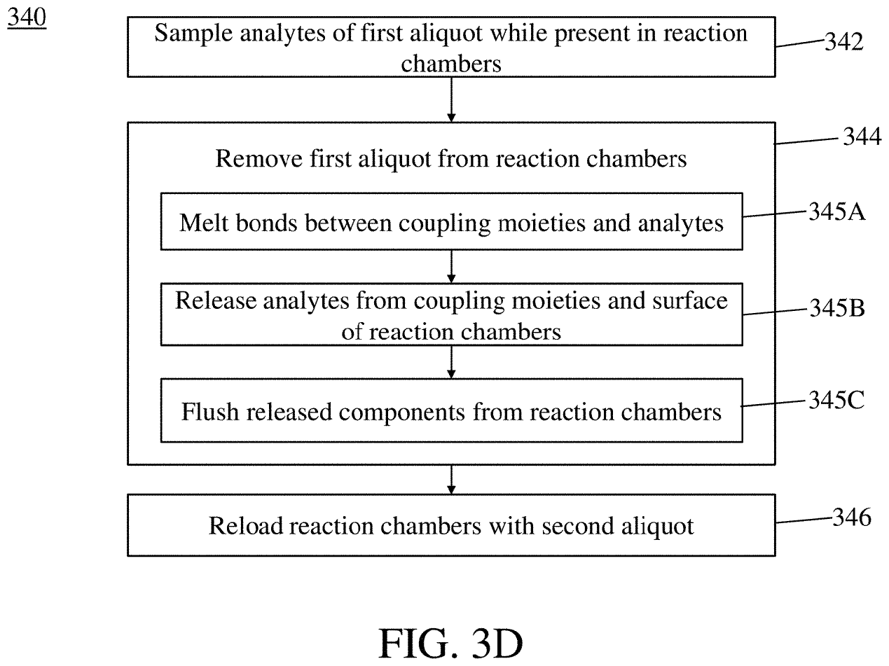

FIG. 3A is an example process 310 for regenerating the surface of a chamber of the integrated device of FIG. 1A, according to some embodiments. Process 310 begins at act 312 where analytes of a first aliquot are sampled while present in reaction chambers of the integrated device. For example, excitation light from at least one light source may be delivered to the reaction chambers and signal emitted from the reaction chambers (e.g., from the analytes of the first aliquot and/or fluorescent labels attached thereto) may be detected at a photodetection region of the integrated device.

Subsequent to sampling, at act 314, the first aliquot may be removed from the reaction chambers. For example, the first aliquot may be removed from the integrated device to allow for loading of a second aliquot of the sample without signal interference from the first aliquot. Act 314 provides one example of unloading the first aliquot from the integrated device which involves disrupting a bond between a coating molecule (e.g., biotin, streptavidin) coating the surface of a reaction chamber and a coupling moiety (e.g., streptavidin, biotin) bound to a sample analyte which has been fixed to the surface of the reaction chamber.

The inventors have recognized that conventional techniques for disrupting a bond between molecules such as biotin and streptavidin are inadequate. Such techniques typically require harsh conditions, including high temperatures (e.g., 90 degrees Celsius) and chemicals which may cause damage to the integrated device. Such techniques may take long periods of time to perform (e.g., greater than 60 minutes) which is incompatible with the desire to continuously process successive aliquots of a sample on a single integrated device.

Thus, the inventors have developed improved techniques for disrupting the bond between a coating molecule and a coupling moiety. An example process for disrupting the coating molecule-coupling moiety bond begins at act 315A. At act 315A, surfaces of the reaction chambers may be contacted with (e.g., soaked in) a regeneration solution. The regeneration solution facilitates disruption of bonds between coating molecules and coupling moieties.

In some embodiments, the regeneration solution comprises an acetate and water. In some embodiments, the acetate is ammonium acetate. Ammonium acetate may improve the solubility of the analyte (e.g., peptide or other biological sample) such that once the analyte is released from the surface of the reaction chamber, the analyte may be dissolved and/or flushed away. In other embodiments, the acetate is sodium acetate. As described herein, existing solutions for disrupting bonds between binding and coating molecules contain harsh chemical which may damage components of the integrated device. Water, by contrast, is compatible with integrated device components and does not carry a risk of causing damage to the integrated device.

The regeneration solution may further comprise a compound (e.g., an organic compound) adapted for disrupting of the coupling moiety and the coating molecule. For example, in some embodiments, the regeneration solution comprises an organic compound for disrupting the biotin-streptavidin complex, such as a fluorinated alcohol (or fluoroalcohol), such as hexafluoro-2-propanol (i.e., 1,1,1,3,3,3-hexafluoro-2-propanol, hexafluoro-iso-propanol, or “HFIP”), or a hexafluoro-2-propanol analog. Such a compound is able to break the bond between the coating molecule and the coupling moiety, thereby allowing the analyte to be released from the surface of the reaction chamber. In some embodiments, the compound may further disrupt the coupling moiety (e.g., streptavidin) complex itself, breaking the bond between the bonding molecule and the analyte.

In some embodiments, the regeneration solution comprises an alcohol, an acetate (e.g., ammonium acetate) and water. In some embodiments, the regeneration solution comprises a fluorinated alcohol, an acetate and water. In some embodiments, the regeneration solution comprises a fluorinated ketone, an acetate and water. In some embodiments, the regeneration solution comprises an alcoholic irritant, an acetate and water. In some embodiments, the regeneration solution further comprises free biotin or an analog thereof, for example, mono-biotin, iminobiotin and/or desthiobiotin.

Free biotin analogs may, in some embodiments, include biotin having a polyethylene glycol (PEG) linker, or another linker, for increasing the solubility of the regeneration solution. Chemical structures of exemplary free biotin analogs are provided below.

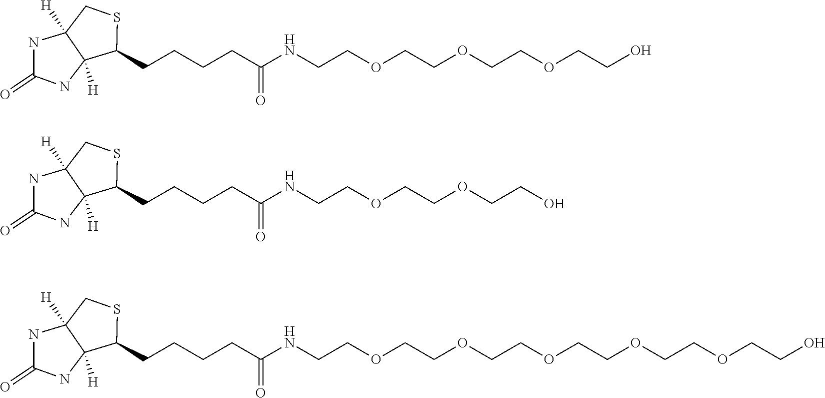 [SMILES: O=C(CCCC[C@@H]1SC[C@@H]2NC(=O)N[C@@H]21)NCCOCCOCCOCCO ; O=C(CCCC[C@@H]1SC[C@@H]2NC(=O)N[C@@H]21)NCCOCCOCCO ; O=C(CCCC[C@@H]1SC[C@@H]2NC(=O)N[C@@H]21)NCCOCCOCCOCCOCCOCCO]

As described herein, existing techniques for disrupting molecules are lengthy, which is not compatible with the desire to continuously process samples. Regeneration solutions having a composition as described herein are capable of or adapted for disrupting coating molecules and coupling moieties in 60 minutes or less, in some embodiments. In some embodiments, the soaking is performed for a discrete period of time of 30 minutes or less. Shorter soaking durations allow for processing multiple aliquots of a sample with a continuous flow. In some embodiments, the regeneration solutions described herein are suitable for automated methods of chip regeneration.

As described herein, existing techniques for disrupting molecules must be performed at high temperatures, for example, 90 degrees Celsius. Regeneration solutions having a composition as described herein are capable of disrupting coating molecules and coupling moieties at room temperature (e.g., at least 20 degrees Celsius and no more than 22 degrees Celsius). Lower operating temperatures reduce the risk of damage to the sample, integrated device, and/or operators which may result from use of high temperatures.

In some embodiments, the regeneration solution comprises Ethylenediaminetetraacetic acid (EDTA). In some embodiments, the regeneration solution comprises formamide. In some embodiments, the regeneration solution comprises EDTA and formamide. In some embodiments, the regeneration solution comprises sodium acetate and formamide. In some embodiments, the regeneration solution contains a fluoroalcohol selected from hexafluoro-2-propanol, nonafluoro-tert-butyl alcohol, 2,2,3,3,4,4,4-heptafluoro-1-butanol, and octafluoro-2-butanone. In some embodiments, the regeneration solution comprises hexafluoro-2-propanol. In some embodiments, the regeneration solution comprises an analog of a hexafluoro-2-propanol, such as nonafluoro-tert-butyl alcohol, 2,2,3,3,4,4,4-heptafluoro-1-butanol, octafluoro-2-butanol, or octafluoro-2-butanone. In some embodiments, the regeneration solution comprises one or more of nonafluoro-tert-butyl alcohol, 2,2,3,3,4,4,4-heptafluoro-1-butanol, and octafluoro-2-butanone, an acetate (e.g., ammonium acetate), and water.

At act 315B, respective coupling moieties are disrupted from coating molecules by breaking a bond between the binding and coating molecules, e.g., by applying a regeneration solution to the chip. For example, as described herein, the regeneration solution may contain a compound for disrupting the bonding molecules from the coupling moieties, such as hexafluoro-2-proponol (e.g., HFIP). HFIP is a non-toxic alcohol having an aromatic odor. HFIP is an irritant, as it is corrosive to the skin and mucous membranes. In some embodiments, the solution contains a hexafluoro-2-propanol in an amount of about 1-5 mL, 3-7 mL, 4-8 mL, 5-9 mL, 5-10 mL, 8-10 mL, 9-11 mL, 10-12.5 mL, or more than 12.5 mL.

In some embodiments, the solution contains HFIP in an amount of about 1, 2, 3, 4, 5, 5.5, 6, 6.5, 7, 7.5, 8, 8.5, 9, 9.5, 10, 10.5, 11, 11.5, 12, 12.5, or more than 12.5 mL. In some embodiments, the solution contains HFIP in an amount of about 1, 2, 3, 4, 5, 5.5, 6, 6.5, 7, 7.5, 8, 8.5, 9, 9.5, 10, 10.5, 11, 11.5, 12, 12.5, or more than 12.5 mL. In some embodiments, the solution contains HFIP in an amount of about 1-15 mL, 1-12.5 mL, 1-5 mL, 3-7 mL, 4-8 mL, 6-9 mL, 5-10 mL, 7-11 mL, 8-10 mL, 9-11 mL, 10-12.5 mL, or more than 12.5 mL. In some embodiments, the solution contains HFIP in an amount of about 9 mL. In some embodiments, the solution contains HFIP in an amount of 9 mL.

The regeneration solution may further contain ammonium acetate (C₂H₄O₂.H₃N). In some embodiments, the solution contains 5M ammonium acetate in an amount of 0.1, 0.2, 0.25, 0.35, 0.4, 0.45, 0.5, 0.55, 0.6, 0.7, 0.8, 0.9, or 1.0 mL. In some embodiments, the solution contains 5M ammonium acetate in an amount of 0.5 mL.

In some embodiments, the solution contains a biotin compound. Addition of a biotin compound may enhance the competition of the regeneration solution for the streptavidin binding sites at the functionalized sample well surface. Addition of the biotin compound may (further or otherwise) facilitate the dissociation of biotin-streptavidin interactions at the surface of the sample well, as mediated by the hexafluoro-2-propanol component of the solution. The solution may contain mono-biotin or bis-biotin. In some embodiments, the solution PEGylated biotin, such as biotin-PEG₃. The PEG linker may enhance the solubility of biotin in the solution. In some embodiments, the solution contains biotin-PEG₃, biotin-PEG₄, biotin-PEG₅, biotin-PEG₆, biotin-PEG₇, or biotin-PEG₈. In some embodiments, the regeneration solution contains a biotin compound at a concentration of between 10 and 500 between 50 and 250 between 100 and 250 between 100 and 500 between 100 and 200 or between 100 and 150 In some embodiments, the regeneration solution contains a biotin compound at a concentration of between 100 μM and 200 μM.

In exemplary embodiments, the regeneration solution contains 9 mL HFIP, 0.5 mL 5M ammonium acetate, 1 mL water, and biotin-PEG₃ (bis-biotin-PEG₄) at a concentration of between 100 μM and 200 μM.

In some embodiments of the regeneration solution, e.g., embodiments for use in identification and/or sequencing of nucleic acid analytes, the regeneration solution contains a DNase enzyme. The DNase enzyme may be provided as part of a nucleoside digestion mix.

In exemplary embodiments, the DNase enzyme (or a nucleoside digestion mix) is applied to the chip separately from the regeneration solution. Thus, in some embodiments, chip regeneration methods provided herein comprise application of the regeneration solution and DNase enzyme together or separately. In some embodiments, the DNase enzyme is applied to the chip before the regeneration solution. Accordingly, in these embodiments, prior to contacting the surface of the sample well with the regeneration solution, a nucleic acid (DNA) analyte is at least partially enzymatically digested with DNase.

In some embodiments, soaking surfaces of the reaction chambers in the regeneration solution further causes the covalent linkage between the coupling moiety and the analyte to be disrupted. As a result of this disrupting, the analytes and coupling moieties are released from the surface of respective reaction chambers at act 315C. The released analytes and coupling moieties may be flushed from the reaction chambers at act 315D. As described herein, the regeneration solution may contain components for increasing the solubility of the analyte, such as ammonium acetate. Thus, in some embodiments, the analyte may additionally or alternatively be dissolved in the regeneration solution.

After removing the first aliquot from the reaction chambers of the integrated device, the process 310 may proceed to act 316. The removal process described herein ensures that there is no interference arising from signals emitted from previously loaded aliquots occurring when sampling subsequently loaded aliquots. Thus, once the first aliquot is removed, a second aliquot may be loaded into reaction chambers of the integrated device at act 316. The second aliquot may comprise analytes, the analytes being bound to respective coupling moieties. The coating molecules coating the surfaces of the reaction chambers may remain in the reaction chambers subsequent to the removal of the first aliquot. Thus, process 310 may not require a recoating step of loading additional coating molecules into the reaction chambers of the integrated device before loading the second aliquot. The coupling moieties of the second aliquot may bind to the remaining coating molecules to fix analytes of the second aliquot to surfaces of reaction chambers such that sampling may be performed.

Subsequent to act 316, analytes of the second aliquot may be sampled. For example, excitation light from at least one light source may be delivered to the reaction chambers and signal emitted from the reaction chambers (e.g., from the analytes of the second aliquot and/or fluorescent labels attached thereto) may be detected at a photodetection region of the integrated device. The above-described process of loading and unloading an integrated device via a technique for disrupting coating molecules and coupling moiety bonds may be repeated as many times as desired.

In some embodiments, the methods comprise disrupting one or more bonds between a coupling moiety and multiple coating molecules, such as a first coating molecule and a second coating molecule, wherein the first and second coating molecule is functionalized to the well surface. In some embodiments, the first and the second coating molecule are different. In some embodiments, the first coating molecule is a streptavidin protein and the second coating molecule is not a streptavidin protein (e.g., is an avidin protein or an azide moiety). In some embodiments, the methods comprise disrupting bonds between a coating molecule and multiple types of coupling moieties, such as a first and a second coupling moiety, wherein the coating molecule is functionalized to the well surface. In some embodiments, the first and the second coupling moiety are different. In some embodiments, the first coupling moiety is a biotin moiety and the second coupling moiety is not a biotin moiety (e.g., is a SNAP-tag or an azide moiety). In all such embodiments, use of a regeneration solution in accordance with the disclosed methods may achieve disruption of the bonds between combinations of multiple types of coating molecules and multiple types of coupling moieties.

In some embodiments, prior to contacting the surfaces of the reaction chambers with the regeneration solution at act 315A in order to disrupt the bond between coating molecules and coupling moieties, the analyte may be at least partially enzymatically digested. For example, in some embodiments (e.g., in DNA sequencing applications), the analyte being sampled is considerably large such that the regeneration solution, including the compound adapted for disrupting of the coupling moiety and the coating molecule, may be prevented from contacting the surface of a reaction chamber due to the size of the analyte. As such, in some embodiments, at least a portion of the analyte being sampled in a reaction chamber may be enzymatically digested in order to allow the compound adapted for disrupting of the coupling moiety and the coating molecule to contact the surface of the reaction chamber. For example, where the analyte comprises a DNA strand (e.g., in DNA sequencing), at least a portion of the DNA strand may be enzymatically digested (e.g., using endonuclease (e.g., a DNase) and/or exonuclease digestion) before the surfaces of the reaction chambers are contacted with the regeneration solution.

Stripping Reaction Chamber Coating

A second exemplary technique for unloading a portion of a sample from reaction chambers of the integrated device involves stripping coating molecules from the reaction chamber surfaces. Stripping the coating molecules from the reaction chamber surfaces releases the coupling moieties bound to the coating molecules and analytes attached thereto from the reaction chamber and allows for dissolving and/or flushing the released components from the integrated device.

For example, FIG. 3B is an example process for regenerating the surface of a chamber of the integrated device of FIG. 1A which involves stripping coatings from reaction chambers surfaces, according to some embodiments. Process 320 begins at act 322 where analytes of a first aliquot are sampled while present in reaction chambers of the integrated device. For example, excitation light from at least one light source may be delivered to the reaction chambers and signal emitted from the reaction chambers (e.g., from the analytes of the first aliquot and/or fluorescent labels attached thereto) may be detected at a photodetection region of the integrated device.

Subsequent to sampling, at act 324, the first aliquot may be removed from the reaction chambers. For example, the first aliquot may be removed from the integrated device to allow for loading of a second aliquot of the sample without signal interference from the first aliquot. Act 324 provides a second example of unloading the first aliquot from the integrated device which involves removing a coating comprising one or more coating molecules (e.g., biotin, streptavidin) from a surface of a reaction chamber.

In particular, at act 325A, one or more coating molecules coating surfaces of the reaction chambers are stripped from the reaction chamber surfaces. In some embodiments, the step of stripping one or more coating molecules comprises the addition of any regeneration solution as disclosed above. In some embodiments, this step comprises the addition of HFIP, or 1,1,1,3,3,3-Hexafluoro-2-Propanol, solution.

As described herein, coupling moieties are bound to the coating molecules in the reaction chambers to fix a sample analyte of the first aliquot which are bound to the coupling moieties to a surface of the reaction chamber to enable sampling. Thus, as a result of removing the coating, the analytes and coupling moieties are released from the surface of respective reaction chambers as well, at act 325B. The released analytes and coupling moieties may be flushed from the reaction chambers at act 325C. In some embodiments, the released components may additionally or alternatively be dissolved, for example, by soaking the surface of the chip in a solution, such as any of the regeneration solutions described herein. In some embodiments, the disclosed methods comprise the stripping of a first coating molecule and a second coating molecule from the reaction chamber surface, wherein the first and second coating molecule are different. In such embodiments, the first coating molecule may comprise a streptavidin protein and the second coating molecule may comprise a moiety that is not a streptavidin protein (e.g., may be an avidin protein or an azide moiety).

After removing the first aliquot from the reaction chambers of the integrated device, the process 320 may proceed to act 326. At act 326, surfaces of the reaction chambers are recoated by loading additional coating molecules (e.g., biotin, streptavidin) into the reaction chambers to prepare for the loading of a second aliquot.

At act 328, the reaction chambers of the integrated device may be reloaded with a second aliquot of the sample. Subsequent to act 328, analytes of the second aliquot may be sampled. The above-described process of loading and unloading an integrated device via a technique for stripping reaction chamber coatings may be repeated as desired.

Enzymatically Digesting Analytes

A third exemplary technique for unloading a portion of a sample from reaction chambers of the integrated device involves enzymatically digesting analytes (e.g., peptides) present in the reaction chambers subsequent to sampling. Enzymatically digesting analytes subsequent to sampling allows for removing the previously sampled analytes from reaction chambers without requiring disrupting of coating molecules and coupling moieties or recoating surfaces of the reaction chambers of the integrated device.

For example, FIG. 3C is an example process for regenerating the surface of a chamber of the integrated device of FIG. 1A which involves enzymatically digesting previously sampled analytes, according to some embodiments. Process 330 begins at act 332 where analytes of a first aliquot are sampled while present in reaction chambers of the integrated device. For example, excitation light from at least one light source may be delivered to the reaction chambers and signal emitted from the reaction chambers (e.g., from the analytes of the first aliquot and/or fluorescent labels attached thereto) may be detected at a photodetection region of the integrated device.

Subsequent to sampling, at act 334, the first aliquot may be removed from the reaction chambers. For example, the first aliquot may be removed from the integrated device to allow for loading of a second aliquot of the sample without signal interference from the first aliquot. Act 334 provides a second example of unloading the first aliquot from the integrated device which involves completely enzymatically digesting previously sampled analytes present in the reaction chambers of the integrated device.

In particular, at act 335A, analytes of the first aliquot may be enzymatically digested. For example, cleavage enzymes, also referred to as cutters, and commonly used in protein and DNA/RNA sequencing, shown in FIG. 2A, may be used to cleave an analyte from its fixed position on the surface of a reaction chamber. Typically, cleavage enzymes are used to cleave a single peptide from a chain of peptides to perform sequencing. However, the inventors have recognized that by soaking the reference chambers in a solution containing a concentration of cleavage enzymes for an extended length of time, an entire peptide chain may be cleaved from its fixed point on the reaction chamber surface. The enzymatically digested analyte may be subsequently flushed and/or dissolved to remove the analytes of the first aliquot from the integrated device.

In some embodiments, each reaction chamber may comprise multiple coating molecules coating the surface of the reaction chamber. Thus, even where a previously bound coating molecule are occupied by previously bound analytes enzymatically digested at act 335A of process 330, additional coating molecules may remain in the reaction chamber to bind to coupling moieties of subsequent aliquots of the sample. In some embodiments, additional coating molecules may be loaded into the chip to recoat the surfaces of the reaction chambers subsequent to act 335.

After removing the first aliquot from the reaction chambers of the integrated device, the process 330 may proceed to act 336. At act 336, the reaction chambers of the integrated device may be reloaded with a second aliquot of the sample. Analytes of the second aliquot may bind via coupling moieties to the remaining coating molecules present in the reaction chambers. Subsequent to act 336, analytes of the second aliquot may be sampled. The above-described process of loading and unloading an integrated device via a technique for enzymatically digesting previously sampled analytes may be repeated as desired.

Melting Analyte-Coupling Moiety Bonds

A fourth exemplary technique for unloading a portion of a sample from reaction chambers of the integrated device involves melting a bond between sample analytes and coupling moieties attached thereto while the sample analytes are present in the reaction chambers. For example, FIG. 3D shows an exemplary process for regenerating the surface of a chamber of the integrated device of FIG. 1A which involves melting a bond formed between sample analytes and coupling moieties, according to some embodiments. Process 340 begins at act 342 where analytes of a first aliquot are sampled while present in reaction chambers of the integrated device. For example, excitation light from at least one light source may be delivered to the reaction chambers and signal emitted from the reaction chambers (e.g., from the analytes of the first aliquot and/or fluorescent labels attached thereto) may be detected at a photodetection region of the integrated device. In some embodiments, the bond formed between the sample analytes and coupling moieties comprises a double-stranded nucleic acid molecule (e.g., a DNA molecule linker in protein sequencing applications), and melting the bond comprises denaturating the doubled-stranded nucleic acid molecule into single strands to release the sample analyte.

Subsequent to sampling, at act 344, the first aliquot may be removed from the reaction chambers. For example, the first aliquot may be removed from the integrated device to allow for loading of a second aliquot of the sample without signal interference from the first aliquot. Act 344 provides a second example of unloading the first aliquot from the integrated device which involves melting a bond formed between sample analytes and coupling moieties configured to bond to coating molecules coating a surface of the reaction chambers.

In particular, at act 335A, bonds between sample analytes and coupling moieties may be melted. In some embodiments, act 335A comprises denaturating a DNA strand linking the sample analyte to the coupling moiety from double strand DNA to single strand DNA to release the analyte. Denaturating a DNA molecule linker formed between the sample analyte and the coupling moiety may comprise injecting energy into the system, such as heat, to melt the DNA strand. In some embodiments, this step comprises addition of any regeneration solution as disclosed above and/or adherence of any other chemical solution.

As described herein, sample analytes are configured to be fixed to the reaction chamber surfaces via a bond formed between coating molecules coating the reaction chamber surfaces and coupling moieties attached to the sample analytes. Process 340 contemplates breaking the bond between the coupling moieties and the sample analytes such that the analytes are no longer fixed to the reaction chamber surfaces. Thus, subsequent to melting the covalent bonds between the coupling moieties and the sample analytes, analytes of the first aliquot may be released from the surface of the reaction chambers at act 345B. The released components may be flushed from the reaction chambers at act 345C. In some embodiments, the released components may additionally and/or alternatively be dissolved.

After removing the first aliquot from the reaction chambers of the integrated device, the process 340 may proceed to act 346. At act 346, the reaction chambers of the integrated device may be reloaded with a second aliquot of the sample. Analytes of the second aliquot may bind via coupling moieties to the remaining coating molecules present in the reaction chambers. Subsequent to act 346, analytes of the second aliquot may be sampled. The above-described process of loading and unloading an integrated device via a technique for melting coupling moiety-analyte bonds may be repeated as desired.

In some embodiments, the step of melting a bond between sample analytes and coupling moieties comprises melting bonds between an analyte and multiple types of coupling moieties, such as a first and a second coupling moiety. In some embodiments, the first and the second coupling moiety are different. In some embodiments, the first coupling moiety is a biotin moiety and the second coupling moiety is not a biotin moiety (e.g., is a SNAP-tag or an azide moiety).

### EXAMPLE 1

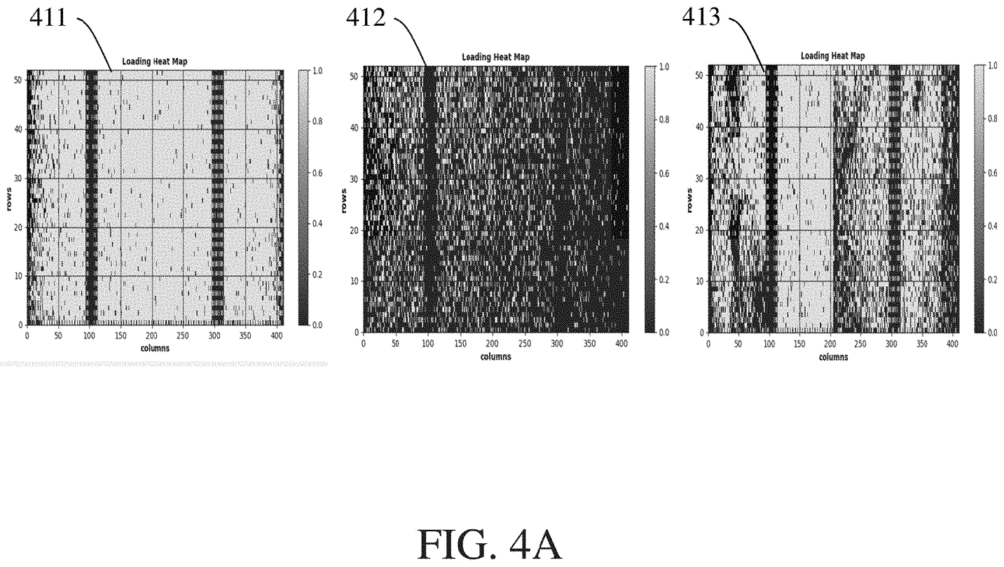

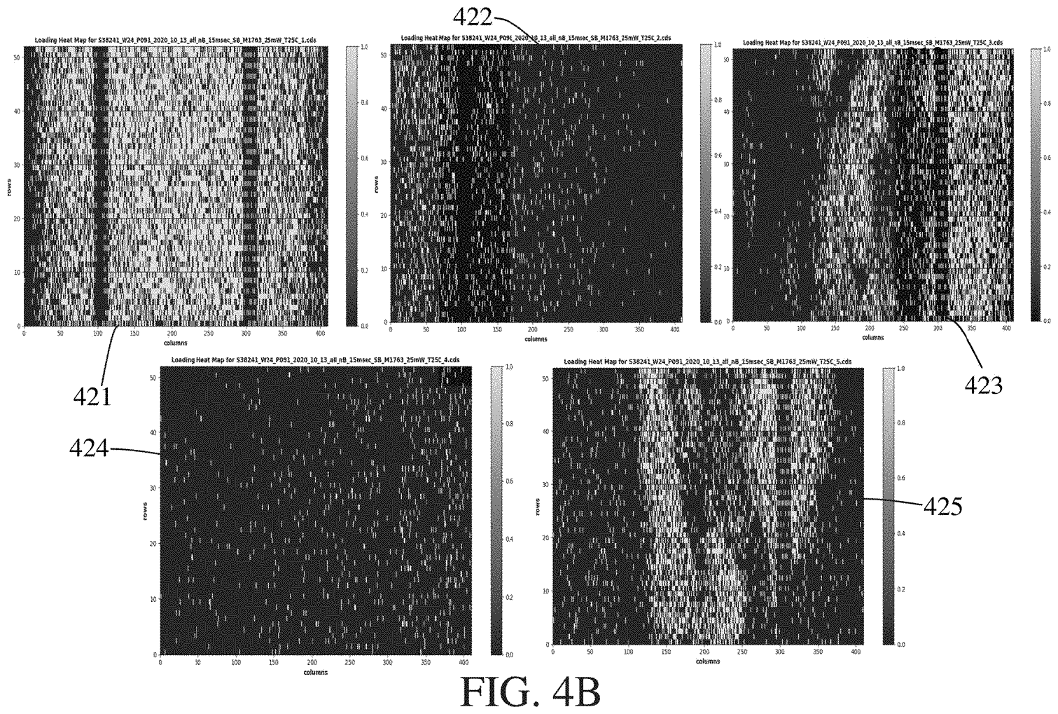

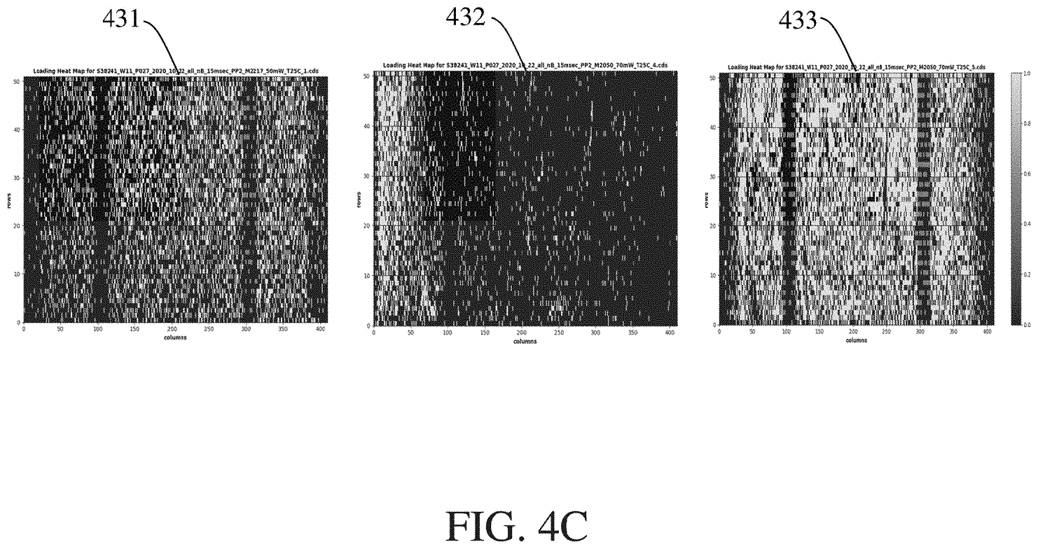

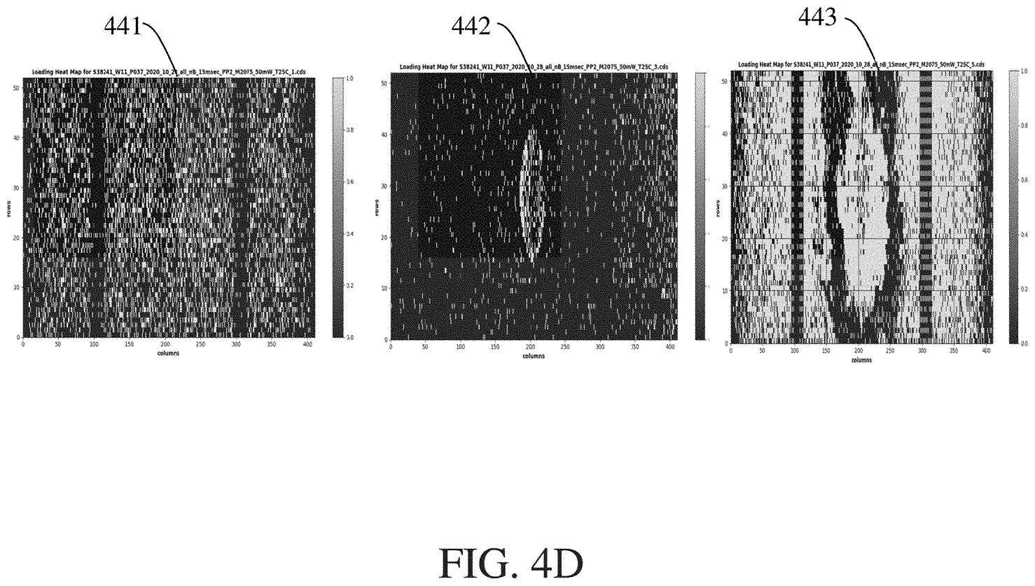

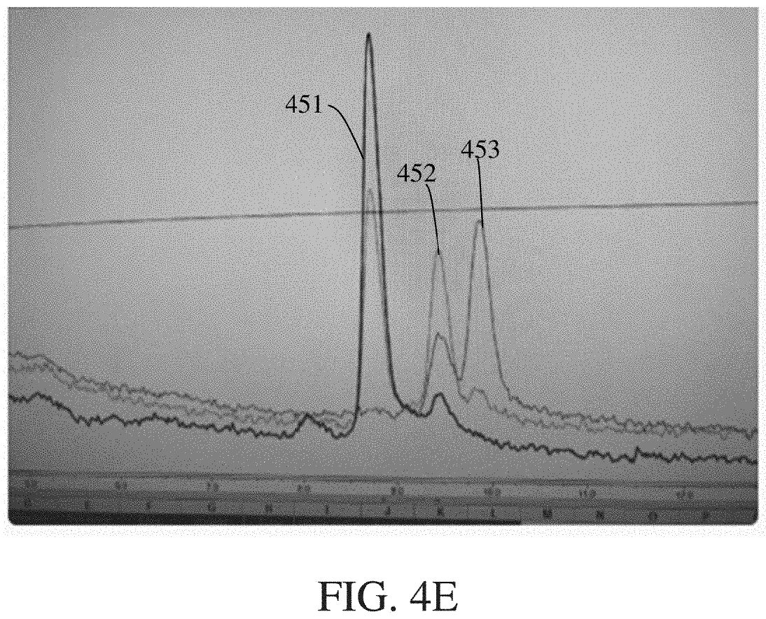

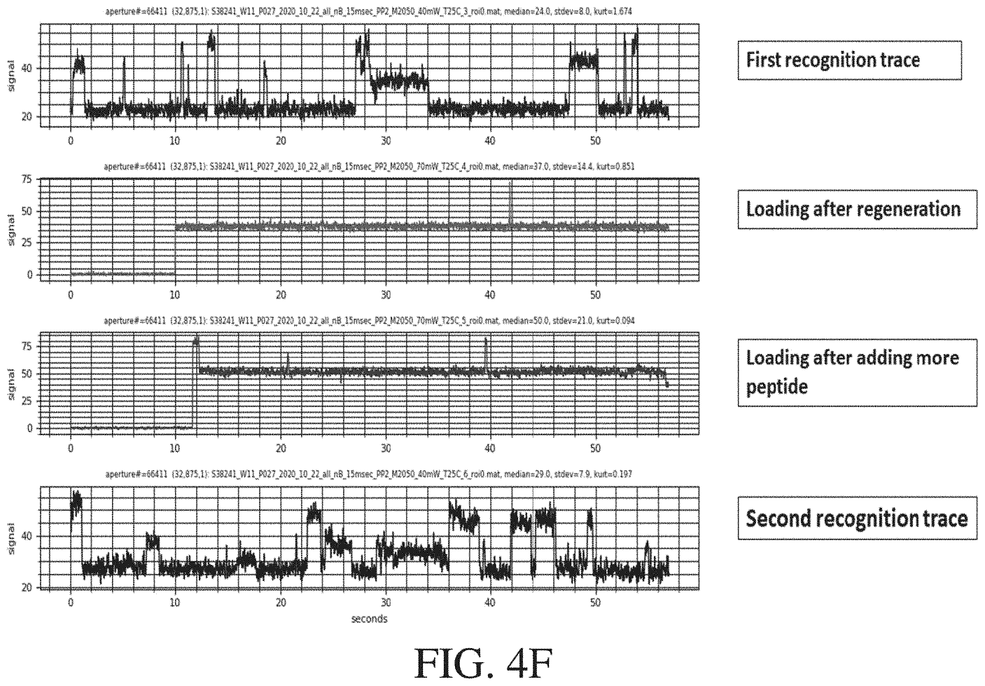

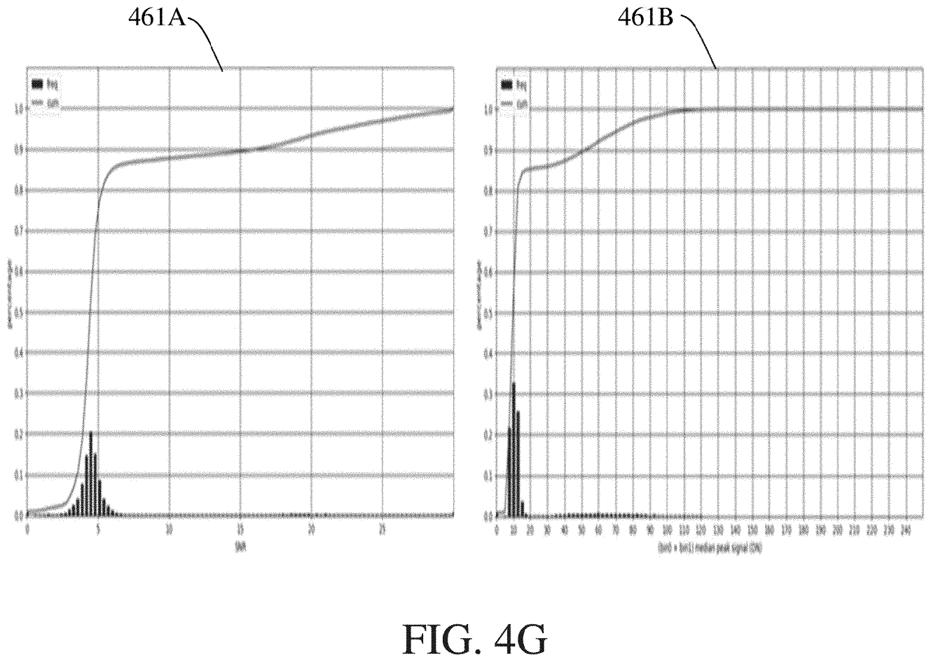

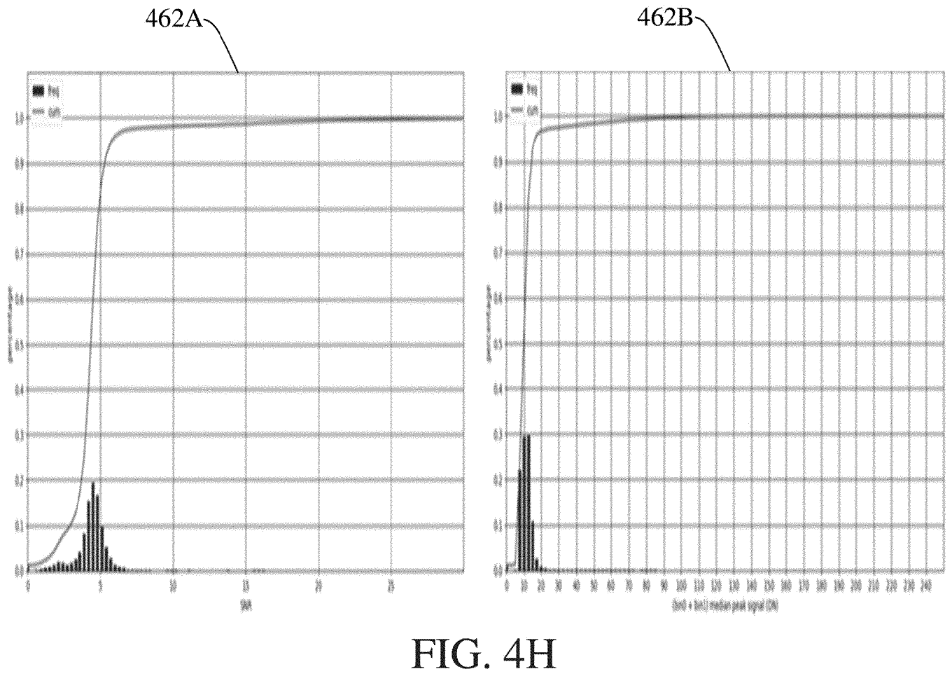

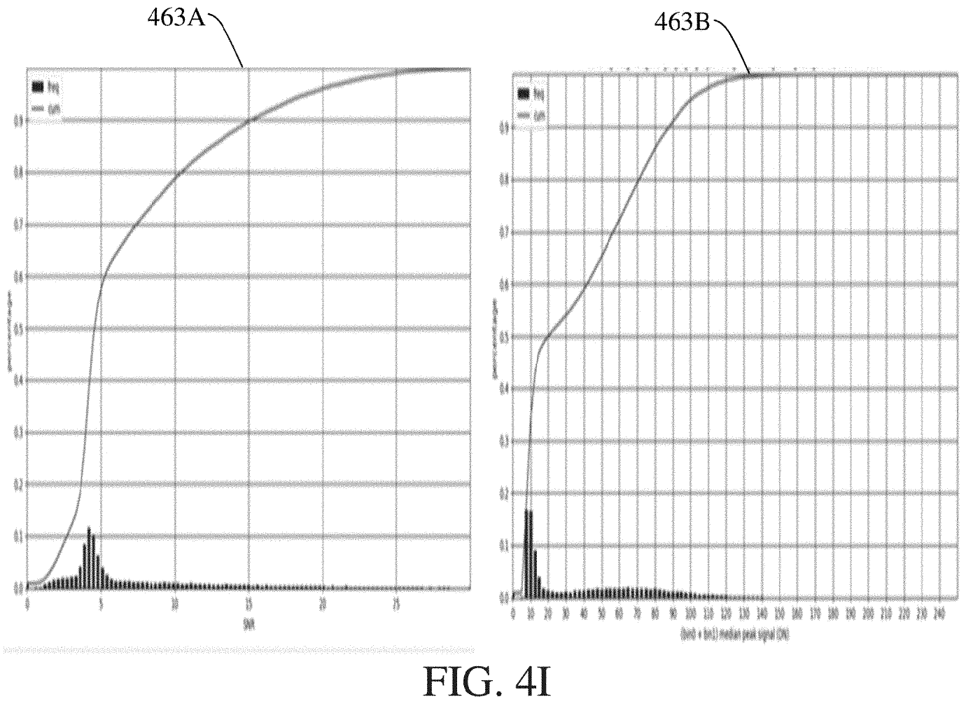

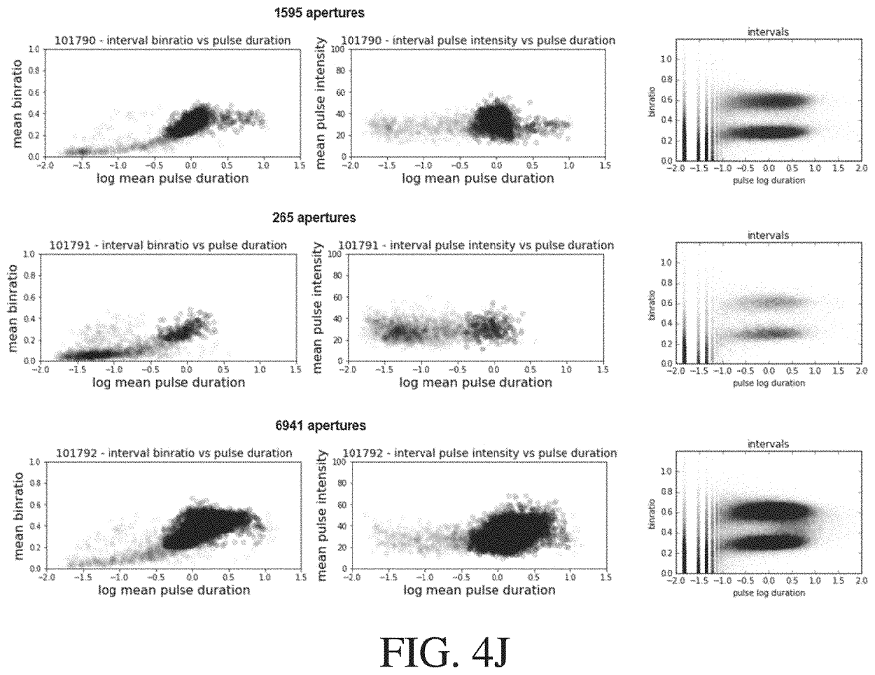

Efficacy of the techniques for removing a portion of a sample from an integrated device is further demonstrated herein, for example, as illustrated in FIGS. 4A-4J. In particular, FIGS. 4A-4J illustrate data accumulated using the chip regeneration techniques for disrupting bonds between coating molecules and coupling moieties. The regeneration solution used in the experiments that generated the following data comprises ammonium acetate, water, and 1,1,1,3,3,3-hexafluoro-2-propanol (HFIP). In particular, the regeneration solution contains 9 mL HFIP, 0.5 mL 5M ammonium acetate, 1mL water, and biotin-PEG₃ at a concentration of between 100 μM and 200 μM.

FIGS. 4A-D illustrate example graphs depicting analyte presence obtained during a process of chip loading and regeneration, according to some embodiments. Taking FIG. 4A for example, a succession of three graphs 411-413 are shown. Graph 411 illustrates signal acquisition while the reaction chambers of the integrated device are 93.6% loaded with analytes. Graph 412 illustrates signal acquisition obtained fifteen minutes after analytes where removed from the reaction chambers according to the disruption techniques described herein (at a 22.5% loading). Graph 413 illustrates signal acquisition subsequent to reloading reaction chambers of the integrated device with additional sample with a 68% loading.

FIGS. 4B-4D illustrate additional example graphs depicting analyte presence obtained during processes of loading and unloading an integrated device. As shown in FIG. 4B, graph 421 illustrates signal acquisition after a first loading has been performed with a 58.7% loading, graph 422 illustrates signal acquisition after a subsequent unloading has been performed (at an 8.5% loading), graph 423 illustrates signal acquisition after a second loading has been performed with a 27.7% loading, graph 424 illustrates signal acquisition after a subsequent unloading has been performed (at a 3.8% loading), and graph 425 illustrates signal acquisition after a third reloading has been performed with a 23.4% loading. As shown in FIG. 4C, graph 431 illustrates signal acquisition after a first loading has been performed with a 30.4% loading, graph 432 illustrates signal acquisition after a subsequent unloading has been performed (at a 13.8% percent loading), and graph 433 illustrates signal acquisition after a second loading has been performed with a 57.7% loading. As shown in FIG. 4D, graph 441 illustrates signal acquisition after a first loading has been performed with a 27.9% loading, graph 442 illustrates signal acquisition after a subsequent unloading has been performed (at a 7.2% loading), and graph 443 illustrates signal acquisition after second loading has been performed with a 64.9% loading.

FIG. 4E illustrates spectra of samples soaked in regeneration solution under different conditions obtained using high-performance liquid chromatography, according to some embodiments. In particular, curve 451 illustrates signal acquisition from a sample first diluted in water and subsequently mixed with a stripping buffer, curve 452 illustrates signal acquisition from a sample incubated with the stripping buffer for ten minutes and subsequently diluted with water and curve 453 illustrates signal acquisition from a sample incubated with the stripping buffer for 50 minutes and subsequently diluted with water.

FIG. 4F illustrates example graphs illustrating signals acquired from reaction chambers of an integrated device in between regeneration and loading steps, according to some embodiments. In particular, a first graph illustrates signal acquisition in response to a recognition pulse delivered to the integrated device while unloaded. A second graph illustrates signal acquisition subsequent to a first regeneration and loading step. A third graph illustrates signal acquisition subsequent to additional loading of the integrated device. A fourth graph illustrates signal acquisition subsequent to unloading and regeneration of the integrated device.

FIGS. 4G-4I illustrate additional example graphs illustrating signals acquired from reaction chambers of an integrated device in between regeneration and loading steps, according to some embodiments. As shown in FIG. 4G, graphs 461A and 461B illustrate signal acquisition from reaction chambers of an integrated device with a 28% sample loading. Graphs 462A and 462B illustrate signal acquisition from reaction chambers of an integrated device with a 7% sample loading. Graphs 463A and 463B illustrate signal acquisition from reaction chambers of an integrated device with a 65% sample loading.

FIG. 4J illustrates additional example graphs illustrating signals acquired from reaction chambers of an integrated device in between regeneration and loading steps, according to some embodiments. In particular, a first row of graphs illustrates signal acquisition subsequent to a first sample loading of the integrated device, a second row of graphs illustrates signal acquisition subsequent to a first unloading of the integrated device, and a third row of graphs illustrates signal acquisition subsequent to a reloading of the integrated device.

IV. Aspects Related to Chip Reuse Techniques

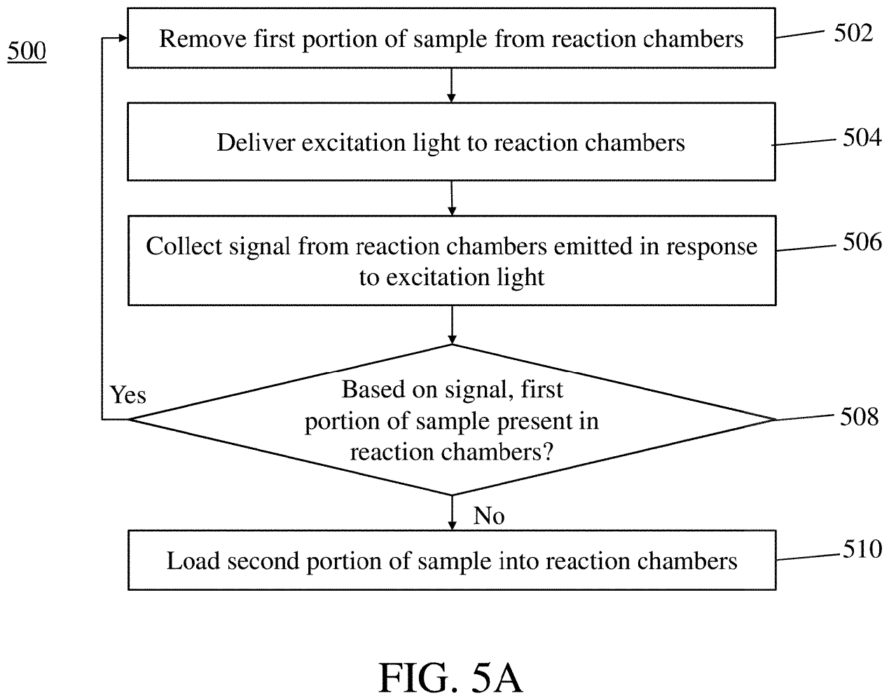

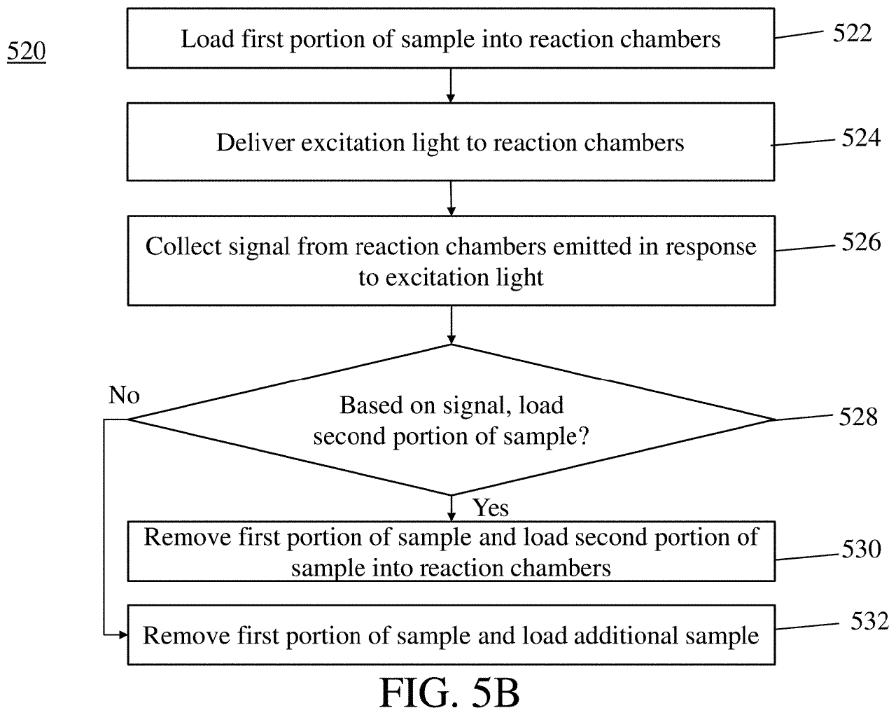

The inventors have further developed additional techniques that may be used in combination with the chip reuse techniques described herein. For example, FIG. 5A is an example process for determining whether a sample is present in a chamber of the integrated device of FIG. 1A, according to some embodiments. The inventors have recognized that it is advantageous to determine whether the unloading techniques described herein have been performed successfully before loading the integrated device with additional sample.

Process 500 shown in FIG. 5A begins at act 502, where a first portion of sample is removed from reaction chambers of an integrated device. For example, act 502 may be performed according to any of the techniques for removing sample from reaction chambers of an integrated device described herein. The first portion of the sample may comprise a first aliquot of a sample having been divided into a plurality of aliquots.

Subsequent to removing the first portion of the sample from the integrated device, a recognition process may be performed whereby recognition pulses of excitation light are used to determine whether there is any sample remaining in the reaction chambers of the integrated device, or whether the sample has been sufficiently removed at act 502. Thus, at act 504, excitation light from at least one light source is delivered to the reaction chambers of the integrated device.

If any analytes of the first portion of the sample remain in the reaction chambers subsequent to act 502, the remaining analytes and/or fluorescent labels attached thereto may become excited in response to the delivery of excitation light from the at least one light source at act 504. The excited analytes and/or fluorescent labels attached thereto may emit photons in response to the excitation which may be collected by a photodetection region of the integrated device at act 504. In addition, the coating molecules, coupling moieties, and/or fluorescent labels attached thereto may emit signal in response to the excitation light. Thus, in some embodiments, any signals received from the reaction chambers by the photodetection region of the integrated device may be used to determine whether any coating molecules and/or coupling moieties are present in the reaction chambers of the integrated device.

At act 508 it is determined whether any of the first portion of the sample is present in the reaction chambers of the integrated device based on the signal collected at act 506. For example, the emitted signal may contain information regarding the analyte, coating molecule, coupling moiety and/or fluorescent label attached thereto from which it was emitted (e.g., luminescent intensity, luminescent lifetime, wavelength, pulse duration, interpulse duration). The integrated device may be configured to use the emissions information to determine what, if anything, is present in the reaction chambers.

If, at act 508, it is determined that at least some of the first portion of the sample is still present in the reaction chambers, the process 500 may proceed through the yes branch to act 502 where a removal process for unloading the first portion of the sample from the reaction chambers is repeated. In some embodiments, the removal process performed upon returning through the yes branch to act 502 may differ from an initial removal process performed at the initiation of process 500.

If, at act 508, it is determined, based on the signal collected at act 506, that the first portion of the sample is no longer present in the reaction chambers, the process 500 may proceed through the yes branch to act 510 where a second portion of the sample is loaded into the reaction chambers of the integrated device. The second portion of the sample may comprise a second aliquot of the plurality of aliquots that the sample is divided into.

The process 500 therefore may be used to determine when reloading of the integrated device with additional sample should be performed. As described herein, the inventors have recognized that previously sampled analytes present in the reaction chamber when newly loaded analytes are being sampled may interfere with signal collection from the newly loaded analytes. Thus, the process 500 may be used to confirm that the previously sampled analytes have been sufficiently removed before proceeding to reload the integrated device.

In some embodiments, at act 508, determining whether the first portion of the sample is present in the reaction chambers comprises determining an amount of the first sample remaining in the reaction chambers. In such embodiments, the determined amount may be compared to a threshold value. If the determined amount is below the threshold value, the process 500 may proceed to act 510 where additional sample is loaded into the reaction chambers of the integrated device. If instead, at act 508, the determined amount is not below the threshold value, the process 500 may refrain from loading additional sample into the reaction chambers.

As described herein, the signal collected at act 506 may be used to determine the presence of coating molecules and/or coupling moieties in the reaction chambers. In such embodiments, the collected signal may be used to determine whether to load additional coating molecules and/or coupling moieties into the reaction chambers of the integrated device, e.g., before loading a second portion of the sample into the reaction chambers of the integrated device.

The inventors have further developed techniques which can be used in combination with the chip reuse techniques described herein for determining whether to continue sampling subsequent aliquots of a sample that has been divided into a plurality of aliquots. For example, the inventors have recognized that some samples may contain information of interest while others need not be fully sampled. Thus, information regarding a sample may be collected from a first aliquot of a plurality of aliquots of a sample and used to determine whether to continue processing the sample by loading a second aliquot of the sample into the integrated device.

For example, FIG. 5B is an example process for determining whether to continue processing a sample with the integrated device of FIG. 1A, according to some embodiments. Process 500 shown in FIG. 5B begins at act 522, where a first portion of a sample is loaded into reaction chambers of an integrated device. For example, the first portion of the sample may comprise a first aliquot of the sample, where the sample has been divided into a plurality of aliquots. The first portion of the sample may comprise a plurality of analytes to be sampled. The analytes may be bound to a coupling moiety as described herein, for fixing the analytes to surfaces of the reaction chambers via bond between the coupling moieties and coating molecules coating the surfaces of the reaction chambers. The analytes may be labeled with a fluorescent dye, in some embodiments.

At acts 524-526 the first portion of the sample loaded into the reaction chambers at act 522 may be sampled. For example, at act 524, excitation light from at least one light source may be delivered to the first portion of the sample while present in the reaction chambers of the integrated device. As a result, analytes of the first portion of the sample and/or fluorescent labels attached thereto may become excited and emit photons collected by a photodetection region of the integrated device at act 526.

At act 528, it is determined, based on the signals collected at act 526, whether to load a second portion of the sample into reaction chambers of the integrated device. For example, as described herein, the emissions signals may comprise information which may be extracted by the integrated device, such as characteristic intensity, wavelength, luminescent lifetime, pulse duration, interpulse duration, etc. In some embodiments, the extracted information may be used to determine an identity of the analytes present in the first portion of the sample. Based on the identity of the analytes, it may be determined that the sample as a whole is of interest, and further processing of additional aliquots of the sample is desirable. In such instances, the process 520 may proceed through the yes branch to act 530.

In other instances, based on the determined identity of the analyte, it may be determined that the sample as a whole is not of interest, and that time and other resources need not be spent further processing a sample. Thus, if, at act 528, it is determined not to load a second portion of the sample, the process 520 may proceed through the no branch to act 532. At act 532, a user may refrain from loading a second portion of the sample into reaction chambers of the integrated device. Instead, the first portion of the sample may be removed from the reaction chambers of the integrated device. In some embodiments, at act 532, a first portion of a second, additional sample may be loaded into reaction chambers of the integrated device for sampling. In some embodiments, a sensor chip of the integrated device may additionally or alternatively be disposed of.

If, at act 528, it is determined that a second portion of the sample should be loaded, the process 520 may proceed through the yes branch to act 530 where a first portion of the sample is removed, for example according to any of the techniques described herein, and a second portion of the sample is loaded into reaction chambers of the integrated device. Subsequent to act 530, the process 520 may be repeated as desired, by returning to act 524 where the second portion of the sample is processed.

V. Increasing Longevity of the Chip

As described herein, the inventors have recognized that in reusing the integrated device for multiple experiments, components of the integrated device may deteriorate over the extended use of the sensor chip. Accordingly, some aspects of the technology described herein relate to techniques for increasing the longevity of use of the integrated device. For example, components of the integrated device may be susceptible to corrosion due to the repeated soaking of the reaction chambers in regeneration solution. Therefore, in some embodiments, components of the integrated device may be configured to resist such deterioration (e.g., including a corrosion resistant coating, etc.). In some embodiments, components of the integrated device may be configured to resist interference with the luminescent signal generated the sequencing workflow that may arise with sequential regeneration of the chip and/or flow cell.

For example, in some embodiments, increasing the longevity of the integrated device chip may be facilitated by maintaining the stability of the surface coating of the reaction chambers of the integrated device. Examples of suitable techniques for maintaining the stability of the surface coating of reaction chambers of the integrated device are described in U.S. patent application Ser. No. 17/067,184, filed Oct. 9, 2020, titled “SURFACE MODIFICATION IN THE VAPOR PHASE” under Attorney Docket Number R0708.70066US01, which is incorporated by reference in its entirety. In some embodiments, the PH value of the regeneration solutions described herein may be maintained at 7 to increase the stability of reaction chamber surface coatings.

VI. Additional Device Components

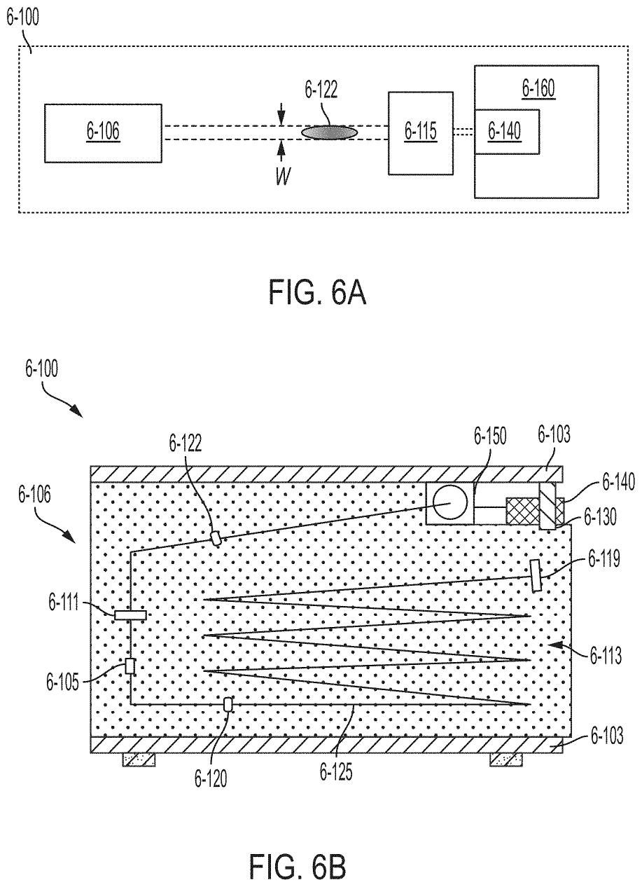

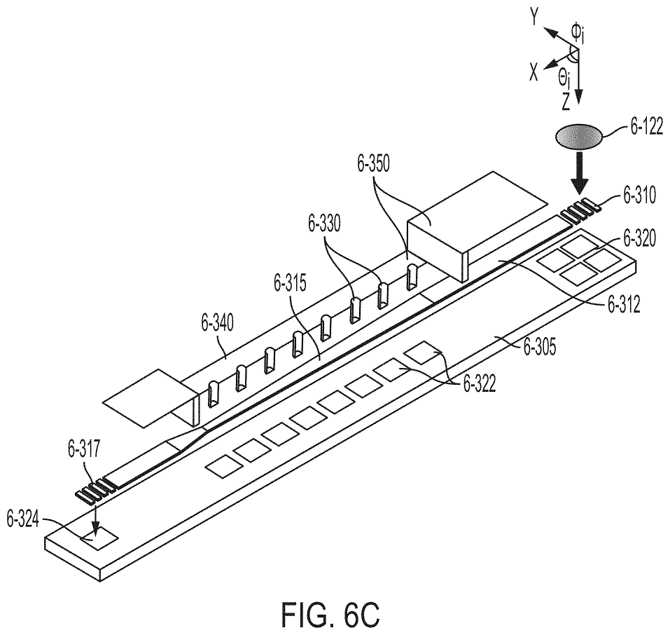

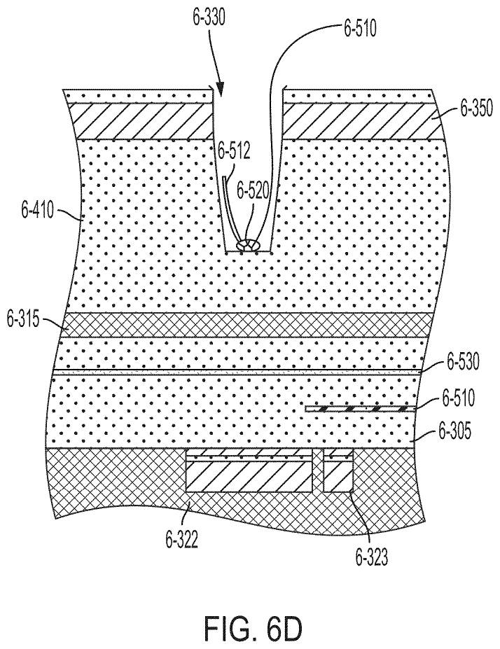

According to some aspects, the integrated device described herein may be configured having one or more additional components. For example, FIG. 6A is a block diagram of an analytical instrument that includes a compact mode-locked laser module, according to some embodiments. As shown in FIG. 6A, a portable, advanced analytic instrument 6-100 can comprise one or more pulsed optical sources 6-106 mounted as a replaceable module within, or otherwise coupled to, the instrument 6-100. The portable analytic instrument 6-100 can include an optical coupling system 6-115 and an analytic system 6-160. The optical coupling system 6-115 can include some combination of optical components (which may include, for example, none, one from among, or more than one component from among the following components: lens, mirror, optical filter, attenuator, beam-steering component, beam shaping component) and be configured to operate on and/or couple output optical pulses 6-122 from the pulsed optical source 6-106 to the analytic system 6-160. The analytic system 6-160 can include a plurality of components that are arranged to direct the optical pulses to at least one reaction chamber for sample analysis, receive one or more optical signals (e.g., fluorescence, backscattered radiation) from the at least one reaction chamber, and produce one or more electrical signals representative of the received optical signals. In some embodiments, the analytic system 6-160 can include one or more photodetectors and may also include signal-processing electronics (e.g., one or more microcontrollers, one or more field-programmable gate arrays, one or more microprocessors, one or more digital signal processors, logic gates, etc.) configured to process the electrical signals from the photodetectors. The analytic system 6-160 can also include data transmission hardware configured to transmit and receive data to and from external devices (e.g., one or more external devices on a network to which the instrument 6-100 can connect via one or more data communications links). In some embodiments, the analytic system 6-160 can be configured to receive a bio-optoelectronic chip 6-140, which holds one or more samples to be analyzed.

FIG. 6B depicts a further detailed example of a portable analytical instrument 6-100 that includes a compact pulsed optical source 6-108. In this example, the pulsed optical source 6-108 comprises a compact, passively mode-locked laser module 6-113. A passively mode-locked laser can produce optical pulses autonomously, without the application of an external pulsed signal. In some implementations, the module can be mounted to an instrument chassis or frame 6-103, and may be located inside an outer casing of the instrument. According to some embodiments, a pulsed optical source 6-106 can include additional components that can be used to operate the optical source and operate on an output beam from the optical source 6-106. A mode-locked laser 6-113 may comprise an element (e.g., saturable absorber, acousto-optic modulator, Kerr lens) in a laser cavity, or coupled to the laser cavity, that induces phase locking of the laser's longitudinal frequency modes. The laser cavity can be defined in part by cavity end mirrors 6-111, 6-119. Such locking of the frequency modes results in pulsed operation of the laser (e.g., an intracavity pulse 6-120 bounces back-and-forth between the cavity end mirrors) and produces a stream of output optical pulses 6-122 from one end mirror 6-111 which is partially transmitting.

In some cases, the analytic instrument 6-100 is configured to receive a removable, packaged, bio-optoelectronic or optoelectronic chip 6-140 (also referred to as a “disposable chip”). The disposable chip can include a bio-optoelectronic chip, for example, that comprises a plurality of reaction chambers, integrated optical components arranged to deliver optical excitation energy to the reaction chambers, and integrated photodetectors arranged to detect fluorescent emission from the reaction chambers. In some implementations, the chip 6-140 can be disposable after a single use, whereas in other implementations the chip 6-140 can be reused two or more times. When the chip 6-140 is received by the instrument 6-100, it can be in electrical and optical communication with the pulsed optical source 6-106 and with apparatus in the analytic system 6-160. Electrical communication may be made through electrical contacts on the chip package, for example.

In some embodiments and referring to FIG. 6B, the disposable chip 6-140 can be mounted (e.g., via a socket connection) on an electronic circuit board 6-130, such as a printed circuit board (PCB) that can include additional instrument electronics. For example, the PCB 6-130 can include circuitry configured to provide electrical power, one or more clock signals, and control signals to the optoelectronic chip 6-140, and signal-processing circuitry arranged to receive signals representative of fluorescent emission detected from the reaction chambers. Data returned from the optoelectronic chip can be processed in part or entirely by electronics on the instrument 6-100, although data may be transmitted via a network connection to one or more remote data processors, in some implementations. The PCB 6-130 can also include circuitry configured to receive feedback signals from the chip relating to optical coupling and power levels of the optical pulses 6-122 coupled into waveguides of the optoelectronic chip 6-140. The feedback signals can be provided to one or both of the pulsed optical source 6-106 and optical system 6-115 to control one or more parameters of the output beam of optical pulses 6-122. In some cases, the PCB 6-130 can provide or route power to the pulsed optical source 6-106 for operating the optical source and related circuitry in the optical source 6-106.

According to some embodiments, the pulsed optical source 6-106 comprises a compact mode-locked laser module 6-113. The mode-locked laser can comprise a gain medium 6-105 (which can be solid-state material in some embodiments), an output coupler 6-111, and a laser-cavity end mirror 6-119. The mode-locked laser's optical cavity can be bound by the output coupler 6-111 and end mirror 6-119. An optical axis 6-125 of the laser cavity can have one or more folds (turns) to increase the length of the laser cavity and provide a desired pulse repetition rate. The pulse repetition rate is determined by the length of the laser cavity (e.g., the time for an optical pulse to make a round-trip within the laser cavity).

In some embodiments, there can be additional optical elements (not shown in FIG. 6B) in the laser cavity for beam shaping, wavelength selection, and/or pulse forming. In some cases, the end mirror 6-119 comprises a saturable-absorber mirror (SAM) that induces passive mode locking of longitudinal cavity modes and results in pulsed operation of the mode-locked laser. The mode-locked laser module 6-113 can further include a pump source (e.g., a laser diode, not shown in FIG. 6B) for exciting the gain medium 6-105. Further details of a mode-locked laser module 6-113 can be found in U.S. patent application Ser. No. 15/844,469, titled “Compact Mode-Locked Laser Module,” filed Dec. 15, 2017 under Attorney Docket Number R0708.70025US01, which is incorporated herein by reference.

When the laser 6-113 is mode locked, an intracavity pulse 6-120 can circulate between the end mirror 6-119 and the output coupler 6-111, and a portion of the intracavity pulse can be transmitted through the output coupler 6-111 as an output pulse 6-122. Accordingly, a train of output pulses 6-122 can be detected at the output coupler as the intracavity pulse 6-120 bounces back-and-forth between the output coupler 6-111 and end mirror 6-119 in the laser cavity.

According to some implementations, a beam-steering module 6-150 can receive output pulses from the pulsed optical source 6-106 and is configured to adjust at least the position and incident angles of the optical pulses onto an optical coupler (e.g., grating coupler) of the optoelectronic chip 6-140. In some cases, the output pulses 6-122 from the pulsed optical source 6-106 can be operated on by a beam-steering module 6-150 to additionally or alternatively change a beam shape and/or beam rotation at an optical coupler on the optoelectronic chip 6-140. In some implementations, the beam-steering module 6-150 can further provide focusing and/or polarization adjustments of the beam of output pulses onto the optical coupler. One example of a beam-steering module is described in U.S. patent application Ser. No. 15/161,088 titled “Pulsed Laser and Bioanalytic System,” filed May 20, 2016 under Attorney Docket Number R0708.70010US02, which is incorporated herein by reference. Another example of a beam-steering module is described in a separate U.S. patent application Ser. No. 15/842,720, filed Dec. 14, 2017 under Attorney Docket number R0708.70024US01 and titled “Compact Beam Shaping and Steering Assembly,” which is incorporated herein by reference.

FIG. 6C illustrates an example of parallel reaction chambers that can be excited optically by a pulsed laser via one or more waveguides, according to some embodiments. Referring to FIG. 6C, the output pulses 6-122 from a pulsed optical source can be coupled into one or more optical waveguides 6-312 on a bio-optoelectronic chip 6-140, for example. In some embodiments, the optical pulses can be coupled to one or more waveguides via a grating coupler 6-310, though coupling to an end of one or more optical waveguides on the optoelectronic chip can be used in some embodiments. According to some embodiments, a quad detector 6-320 can be located on a semiconductor substrate 6-305 (e.g., a silicon substrate) for aiding in alignment of the beam of optical pulses 6-122 to a grating coupler 6-310. The one or more waveguides 6-312 and reaction chambers or reaction chambers 6-330 can be integrated on the same semiconductor substrate with intervening dielectric layers (e.g., silicon dioxide layers) between the substrate, waveguide, reaction chambers, and photodetectors 6-322.

Each waveguide 6-312 can include a tapered portion 6-315 below the reaction chambers 6-330 to equalize optical power coupled to the reaction chambers along the waveguide. The reducing taper can force more optical energy outside the waveguide's core, increasing coupling to the reaction chambers and compensating for optical losses along the waveguide, including losses for light coupling into the reaction chambers. A second grating coupler 6-317 can be located at an end of each waveguide to direct optical energy to an integrated photodiode 6-324. The integrated photodiode can detect an amount of power coupled down a waveguide and provide a detected signal to feedback circuitry that controls the beam-steering module 6-150, for example.

The reaction chambers 6-330 or reaction chambers 6-330 can be aligned with the tapered portion 6-315 of the waveguide and recessed in a tub 6-340. There can be photodetectors 6-322 located on the semiconductor substrate 6-305 for each reaction chamber 6-330. In some embodiments, a semiconductor absorber (shown in FIG. 6-5 as an optical filter 6-530) may be located between the waveguide and a photodetector 6-322 at each pixel. A metal coating and/or multilayer coating 6-350 can be formed around the reaction chambers and above the waveguide to prevent optical excitation of fluorophores that are not in the reaction chambers (e.g., dispersed in a solution above the reaction chambers). The metal coating and/or multilayer coating 6-350 may be raised beyond edges of the tub 6-340 to reduce absorptive losses of the optical energy in the waveguide 6-312 at the input and output ends of each waveguide.

There can be a plurality of rows of waveguides, reaction chambers, and time-binning photodetectors on the optoelectronic chip 6-140. For example, there can be 128 rows, each having 512 reaction chambers, for a total of 65,536 reaction chambers in some implementations. Other implementations may include fewer or more reaction chambers, and may include other layout configurations. Optical power from the pulsed optical source 6-106 can be distributed to the multiple waveguides via one or more star couplers or multi-mode interference couplers, or by any other means, located between an optical coupler 6-310 to the chip 6-140 and the plurality of waveguides 6-312.

A non-limiting example of a biological reaction taking place in a reaction chamber 6-330 is depicted in FIG. 6D. The example depicts sequential incorporation of nucleotides or nucleotide analogs into a growing strand that is complementary to a target nucleic acid. The sequential incorporation can take place in a reaction chamber 6-330, and can be detected by an advanced analytic instrument to sequence DNA. The reaction chamber can have a depth between about 150 nm and about 250 nm and a diameter between about 80 nm and about 160 nm. A metallization layer 6-540 (e.g., a metallization for an electrical reference potential) can be patterned above a photodetector 6-322 to provide an aperture or iris that blocks stray light from adjacent reaction chambers and other unwanted light sources. According to some embodiments, polymerase 6-520 can be located within the reaction chamber 6-330 (e.g., attached to a base of the chamber). The polymerase can take up a target nucleic acid 6-510 (e.g., a portion of nucleic acid derived from DNA), and sequence a growing strand of complementary nucleic acid to produce a growing strand of DNA 6-512. Nucleotides or nucleotide analogs labeled with different fluorophores can be dispersed in a solution above and within the reaction chamber.

VII. Applications for Chip Reuse Techniques

Having thus described multiple techniques for reuse of a sensor chip in order to increase the number of samples processed by a single device, example applications of the chip reuse techniques will now be described. For example, the inventors have recognized that identification of one or more molecules in a sample under analysis may be performed in combination with the techniques described herein. In particular, measurements for one or more characteristics of emission light may be obtained by a device, such as the integrated device described herein, and the collected measurements may be compared to known values of the measured characteristics for a fluorescent marker to determine which fluorescent marker is the most likely source of the emission light. In turn, by identifying the fluorescent marker, the identity of the molecule to which the fluorescent marker is attached can be known based on the particular type of molecule to which the fluorescent marker is known to attach.

The techniques described herein may be used in combination with techniques for detection and/or identification of molecules in a sample, for example, including those described in U.S. patent application Ser. No. 16/686,028 titled “METHODS AND COMPOSITIONS FOR PROTEIN SEQUENCING,” filed Nov. 15, 2019 under Attorney Docket No. R0708.70042US02, PCT Application No. PCT/US19/61831 titled “METHODS AND COMPOSITIONS FOR PROTEIN SEQUENCING,” filed Nov. 15, 2019 under Attorney Docket No. R0708.70042WO00, and U.S. patent application Ser. No. 17/190,331 titledl “INTEGRATED SENSOR FOR MULTI-DIMENSIONAL SIGNAL ANALYSIS,” filed Mar. 2, 2021 under Attorney Docket No. R0708.70090US01, each of which are incorporated by reference in their entireties. Such techniques may be used in applications for protein sequencing and/or nucleic acid (e.g., DNA and/or RNA) sequencing, for example.

Nucleic Acid Sequencing

Some aspects of the disclosure are useful for sequencing biological polymers, such as nucleic acids and proteins. In some embodiments, methods, compositions, and devices described in the disclosure can be used to identify a series of nucleotide or amino acid monomers that are incorporated into a nucleic acid or protein (e.g., by detecting a time-course of incorporation of a series of labeled nucleotide or amino acid monomers) . In some embodiments, the methods and devices described in the disclosure can be used to identify a series of nucleotides that are incorporated into a template-dependent nucleic acid sequencing reaction product synthesized by a polymerase enzyme.

In some embodiments, nucleic acid sequencing comprises providing a nucleic acid of interest that is coated to a surface of a solid support (e.g., attached to a bottom surface of a sample well) through a coupling moiety and/or a coating molecule. In some embodiments, the coupling moiety-coating molecule conjugate is formed by a linkage between a biotin coupling moiety and a streptavidin coupling moiety coated to the surface of a well.

In some aspects, a method of sequencing a target nucleic acid is provided. In some embodiments, the method of sequencing a target nucleic acid comprises steps of: (i) providing a mixture comprising (a) said target nucleic acid, (b) a primer complementary to said target nucleic acid, (c) a nucleic acid polymerase, and (d) nucleotides for incorporation into a growing nucleic acid strand complementary to said target nucleic acid, wherein said nucleotides include different types of luminescently labeled nucleotides, wherein said luminescently labeled nucleotides yield detectable signals during sequential incorporation into said growing nucleic acid strand, which detectable signals for said different types of luminescently labeled nucleotides are differentiable from one another in a time domain (e.g., by determining timing and/or frequency of the detectable signals); (ii) subjecting said mixture of (i) to a polymerization reaction under conditions that are sufficient to yield said growing nucleic acid strand by extension of the primer; (iii) measuring said detectable signals from said luminescently labeled nucleotides following a sequential incorporation into said growing nucleic acid strand; and (iv) determining the timing and/or frequency of said measured detectable signals from said luminescently labeled nucleotides upon sequential incorporation into said growing nucleic acid strand to identify a time sequence of incorporation of said luminescently labeled nucleotides into said growing nucleic acid strand, thereby determining a sequence of said target nucleic acid.

In some aspects, methods of sequencing are provided that comprise steps of: (i) exposing a complex in a target volume of a sample well to one or more labeled nucleotides, the complex comprising a target nucleic acid or a plurality of nucleic acids present in a sample, at least one primer complementary to said target nucleic acid, and a DNA polymerase; (ii) subjecting the complex to a polymerization reaction under conditions that are sufficient to yield a growing nucleic acid strand by extension of the primer; (iii) directing a series of pulses of excitation light towards the target volume; (iv) detecting a plurality of emitted photons from the one or more labeled nucleotides following a sequential incorporation of nucleotides into the growing nucleic acid comprising the primer; and (v) identifying the sequence of incorporated nucleotides by determining one or more characteristics of the emitted photons. These characteristics may be selected from luminescent lifetime, retention time, luminescent intensity, luminescent wavelength, pulse duration, and/or interpulse duration. In some embodiments, the characteristic is luminescent lifetime.

In some embodiments, the free nucleotides are conjugated to the same type of luminescent label, such that detectable signals for the luminescently labeled nucleotides are differentiable from one another in a time domain (e.g., by determining timing, intensity and/or frequency of the detectable signals). In exemplary embodiments, free nucleotides are conjugated to the same type of label, and the differences in intensities of the detectable signal is measured within a time domain. Nucleotides incorporated into a growing nucleic acid strand complementary to the target nucleic acid by the polymerase will remain in the detection region for longer durations than free nucleotides, and as such their intensities will be larger and/or longer than unincorporated nucleotides.

In some embodiments, the free nucleotides (e.g., four types of nucleotides) are each conjugated to a different type of luminescent label, such that detectable signals for the luminescently labeled nucleotides are differentiable from one another in a time domain (e.g., by determining timing, intensity and/or frequency of the detectable signals). Nucleotides incorporated into a growing nucleic acid strand complementary to the target nucleic acid by the polymerase will remain in the detection region for longer durations than free nucleotides, and as such their intensities will be larger and/or longer than unincorporated nucleotides.

Embodiments of the chip regeneration methods provided here are capable of sequencing single nucleic acid molecules with high accuracy and long read lengths, such as an accuracy of at least about 50%, 60%, 70%, 75%, 80%, 85%, 90%, 92%, 95%, 96%, 97%, 98%, 99%, 99.9%, 99.99%, 99.999%, or 99.9999%, and/or read lengths greater than or equal to about 10 base pairs (bp), 50 bp, 100 bp, 200 bp, 300 bp, 400 bp, 500 bp, 1000 bp, 2,500 bp, 5,000 bp, 6,000 bp, 7,000 bp, 7,500 bp, 7,750 bp, 8,000 bp, 8,500 bp, 9,000 bp, 10,000 bp, 20,000 bp, 30,000 bp, 40,000 bp, 50,000 bp, or 100,000 bp. In exemplary embodiments, the disclosed methods are capable of sequencing single nucleic acid molecules with accuracies of about 70%, about 72.5%, about 74%, about 75%, about 80%, about 85%, about 90%, or about 92%. In some embodiments, the disclosed methods are capable of sequencing single nucleic acid molecules with an accuracy of about 94%. In exemplary embodiments, the disclosed methods are capable of sequencing single nucleic acid molecules with read lengths of about 8500 base pairs.

In some embodiments, the target nucleic acid or the nucleic acid polymerase is attached to a support. In some embodiments, the time sequence of incorporation is identified subsequent to subjecting the mixture of (i) to the polymerization reaction. In some embodiments, aspects of the present application can be used to assay biological samples, for example to determine the sequence of one or more nucleic acids or polypeptides in the sample and/or to determine the presence or absence of one or more nucleic acid or polypeptide variants (e.g., one or more mutations in a gene of interest) in the sample. In some embodiments, tests can be performed on patient samples (e.g., human patient samples) to provide nucleic acid sequence information or to determine the presence or absence of one or more nucleic acids of interest for diagnostic, prognostic, and/or therapeutic purposes. In some examples, diagnostic tests can include sequencing a nucleic acid molecule in a biological sample of a subject, for example by sequencing cell free deoxyribonucleic acid (DNA) molecules and/or expression products (e.g., ribonucleic acid (RNA)) in a biological sample of the subject.

In some embodiments, one or more analytes that are being analyzed (e.g., interrogated and/or identified) using luminescent lifetime and/or intensity can be labeled molecules (e.g., analytes that have been labeled with one or more luminescent markers). In some embodiments, analytes comprising one or more biomolecules may be identified using markers. In some examples, luminescent markers are used to identify individual subunits of biomolecules. Some embodiments use luminescent markers (also referred to herein as “markers”), which may be exogenous or endogenous markers. Exogenous markers may be external luminescent markers used as a reporter and/or tag for luminescent labeling. Examples of exogenous markers may include, but are not limited to, fluorescent molecules, fluorophores, fluorescent dyes, fluorescent stains, organic dyes, fluorescent proteins, species that participate in fluorescence resonance energy transfer (FRET), enzymes, and/or quantum dots. Other exogenous markers are known in the art. Such exogenous markers may be conjugated to a probe or functional group (e.g., molecule, ion, and/or ligand) that specifically binds to a particular target or component. Attaching an exogenous tag or reporter to a probe allows identification of the target through detection of the presence of the exogenous tag or reporter. Examples of probes may include proteins, nucleic acid (e.g., DNA, RNA) molecules, lipids and antibody probes. The combination of an exogenous marker and a functional group may form any suitable probes, tags, and/or labels used for detection, including molecular probes, labeled probes, hybridization probes, antibody probes, protein probes (e.g., biotin-binding probes), enzyme labels, fluorescent probes, fluorescent tags, and/or enzyme reporters.

In some embodiments, the luminescent labels among a set of four nucleotides can be selected from dyes comprising an aromatic or heteroaromatic compound and can be a pyrene, anthracene, naphthalene, acridine, stilbene, indole, benzindole, oxazole, carbazole, thiazole, benzothiazole, phenanthridine, phenoxazine, porphyrin, quinoline, ethidium, benzamide, cyanine, carbocyanine, salicylate, anthranilate, coumarin, fluoroscein, rhodamine, or other like compound. Exemplary dyes include xanthene dyes, such as fluorescein or rhodamine dyes, naphthalene dyes, coumarin dyes, acridine dyes, cyanine dyes, benzoxazole dyes, stilbene dyes, pyrene dyes, phthalocyanine dyes, phycobiliprotein dyes, squaraine dyes, BODIPY dyes, and the like.

In some embodiments, the luminescent labels among a set of four nucleotides comprise Alexa Fluor® 546, Cy®3B, Alexa Fluor® 555 and Alexa Fluor® 555, and the FRET pair Alexa Fluor® 555 and Cy®3.5. In some embodiments, the luminescent labels among a set of four nucleotides comprise Alexa Fluor® 555, Cy®3.5, Alexa Fluor® 546, and DyLight® 554-R1. In some embodiments, the luminescent labels among a set of four nucleotides comprise Alexa Fluor® 555, Cy®3.5, ATTO Rho6G, and DyLight® 554-R1. In some embodiments, the luminescent labels among a set of four nucleotides comprise Alexa Fluor® 555, Cy®3B, ATTO Rho6G, and DyLight® 554-R1. In some embodiments, the luminescent labels among a set of four nucleotides comprise Alexa Fluor® 555, Cy®3B, ATTO 542, and DyLight® 554-R1. In some embodiments, the luminescent labels among a set of four nucleotides comprise Alexa Fluor® 555, Cy®3B, ATTO 542, and Alexa Fluor® 546. In some embodiments, the luminescent labels among a set of four nucleotides comprise Cy®3.5, Cy®3B, ATTO Rho6G, and DyLight® 554-R1.

In certain embodiments, at least one type, at least two types, at least three types, or at least four of the types of luminescently labeled nucleotides comprise a luminescent dye selected from the group consisting of 6-TAMRA, 5/6-Carboxyrhodamine 6G, Alexa Fluor® 546, Alexa Fluor® 555, Alexa Fluor® 568, Alexa Fluor® 610, Alexa Fluor® 647, Aberrior Star 635, ATTO 425, ATTO 465, ATTO 488, ATTO 495, ATTO 514, ATTO 520, ATTO Rho6G, ATTO 542, ATTO 647N, ATTO Rho14, Chromis 630, Chromis 654A, Chromeo™ 642, CF™ 514, CF™ 532, CF™ 543, CF™ 546, CF™ 546, CF™ 555, CF™ 568, CF™ 633, CF™ 640R, CF™ 660C, CF™ 660R, CF™ 680R, Cy®3, Cy®3B, Cy®3.5, Cy®5, Cy®5.5, Dyomics-530, Dyomics-547P1, Dyomics-549P1, Dyomics-550, Dyomics-554, Dyomics-555, Dyomics-556, Dyomics-560, Dyomics-650, Dyomics-680, DyLight® 554-R1, DyLight® 530-R2, DyLight® 594, DyLight® 635-B2, DyLight® 650, DyLight® 655-B4, DyLight® 675-B2, DyLight® 675-B4, DyLight® 680, HiLyte™ Fluor 532, HiLyte™ Fluor 555, HiLyte™ Fluor 594, LightCycler® 640R, Seta™ 555, Seta™ 670, Seta™ 700, Seta™ u 647, and Seta™ u 665, or are of formulae (Dye 101), (Dye 102), (Dye 103), (Dye 104), (Dye 105), or (Dye 106), as described herein.

In some embodiments, at least one type, at least two types, at least three types, or at least four of the types of luminescently labeled nucleotides each comprise a luminescent dye selected from the group consisting of Alexa Fluor® 546, Alexa Fluor® 555, Cy®3B, Cy®3.5, DyLight® 554-R1, Alexa Fluor® 546, Atto Rho6G, ATTO 425, ATTO 465, ATTO 488, ATTO 495, ATTO 514, ATTO 520, ATTO Rho6G, and ATTO 542.

In some embodiments, a first type of luminescently labeled nucleotide comprises Alexa Fluor® 546, a second type of luminescently labeled nucleotide comprises Cy®3B, a third type of luminescently labeled nucleotide comprises two Alexa Fluor® 555, and a fourth type of luminescently labeled nucleotide comprises Alexa Fluor® 555 and Cy®3.5.

In some embodiments, at least one type, at least two types, at least three types, or at least four of the types of luminescently labeled nucleotides comprise a luminescent dye selected from the group consisting of Alexa Fluor® 532, Alexa Fluor® 546, Alexa Fluor® 555, Alexa Fluor® 594, Alexa Fluor® 610, CF™ 532, CF™ 543, CF™ 555, CF™ 594, Cy®3, DyLight® 530-R2, DyLight® 554-R1, DyLight® 590-R2, DyLight® 594, DyLight® 610-B1, or are of formulae (Dye 101), (Dye 102), (Dye 103), (Dye 104), (Dye 105), or (Dye 106).

In some embodiments, a first and second type of luminescently labeled nucleotide comprise a luminescent dye selected from the group consisting of Alexa Fluor® 532, Alexa Fluor® 546, Alexa Fluor® 555, CF™ 532, CF™ 543, CF™ 555, Cy®3, DyLight® 530-R2, DyLight® 554-R1, and a third and fourth type of luminescently labeled nucleotide comprise a luminescent dye selected from the group consisting of Alexa Fluor® 594, Alexa Fluor® 610, CF™ 594, DyLight® 590-R2, DyLight® 594, DyLight® 610-B1, or are of formulae (Dye 101), (Dye 102), (Dye 103), (Dye 104), (Dye 105), or (Dye 106).

In certain embodiments, at least one type, at least two types, at least three types, or at least four of the types of luminescently-labeled nucleotide molecules comprise a luminescent protein selected from the group consisting of TagBFP, mTagBFP2, Azurite, EBFP2, mKalamal, Sirius, Sapphire, T-Sapphire, ECFP, Cerulean, SCFP3A, mTurquoise, mTurquoise2, monomeric Midoriishi-Cyan, TagCFP, mTFP1, EGFP, Emerald, Superfolder GFP, monomeric Azami Green, TagGFP2, mUKG, mWasabi, Clover, mNeonGreen, EYFP, Citrine, Venus, SYFP2, TagYFP, monomeric Kusabira-Orange, mKOK, mKO2, mOrange, mOrange2, mRaspberry, mCherry, mStrawberry, mTangerine, tdTomato, TagRFP, TagRFP-T, mApple, mRuby, mRuby2, mPlum, HcRed-Tandem, mKate2, mNeptune, NirFP, TagRFP657, IFP1.4, iRFP, mKeima Red, LSS-mKatel, LSS-mKate2, mBeRFP, PA-GFP, PAmCherryl, PATagRFP, Kaede (green), Kaede (red), KikGR1 (green), KikGR1 (red), PS-CFP2, mEos2 (green), mEos2 (red), PSmOrange, or Dronpa.

Although the present disclosure makes reference to luminescent markers, other types of markers may be used with devices, systems and methods provided herein. Such markers may be mass tags, electrostatic tags, electrochemical labels, or any combination thereof.

While exogenous markers may be added to a sample, endogenous markers may be already part of the sample. Endogenous markers may include any luminescent marker present that may luminesce or “autofluoresce” in the presence of excitation energy. Autofluorescence of endogenous fluorophores may provide for label-free and noninvasive labeling without requiring the introduction of exogenous fluorophores. Examples of such endogenous fluorophores may include hemoglobin, oxyhemoglobin, lipids, collagen and elastin crosslinks, reduced nicotinamide adenine dinucleotide (NADH), oxidized flavins (FAD and FMN), lipofuscin, keratin, and/or porphyrins, by way of example and not limitation.

The disclosure provides techniques for detecting single molecules using sets of luminescent tags (e.g., luminescent markers, luminescent labels) to label different molecules. Such single molecules may be nucleotides or amino acids having tags. Tags may be detected while bound to single molecules, upon release from the single molecules, or while bound to and upon release from the single molecules. In some examples, tags are luminescent tags. Each luminescent tag in a selected set is associated with a respective molecule. For example, a set of four tags may be used to “label” the nucleobases present in DNA —each tag of the set being associated with a different nucleobase, e.g., a first tag being associated with adenine (A), a second tag being associated with cytosine (C), a third tag being associated with guanine (G), and a fourth tag being associated with thymine (T). Moreover, each of the luminescent tags in the set of tags has different properties that may be used to distinguish a first tag of the set from the other tags in the set. In this way, each tag is uniquely identifiable using one or more of these distinguishing characteristics. By way of example and not limitation, the characteristics of the tags that may be used to distinguish one tag from another may include the emission energy and/or wavelength of the light that is emitted by the tag in response to excitation energy, the wavelength of the excitation light that is absorbed by a particular tag to place the tag in an excited state, and/or the emission lifetime of the tag.

In certain embodiments, the template-dependent nucleic acid sequencing product is carried out by naturally occurring nucleic acid polymerases. In some embodiments, the polymerase is a mutant or modified variant of a naturally occurring polymerase. In some embodiments, the template-dependent nucleic acid sequence product will comprise one or more nucleotide segments complementary to the template nucleic acid strand. In one aspect, the disclosure provides a method of determining the sequence of a template (or target) nucleic acid strand by determining the sequence of its complementary nucleic acid strand.

The term “polymerase,” as used herein, generally refers to any enzyme (or polymerizing enzyme) capable of catalyzing a polymerization reaction. Examples of polymerases include, without limitation, a nucleic acid polymerase, a transcriptase or a ligase. A polymerase can be a polymerization enzyme.

Embodiments directed towards single molecule nucleic acid extension (e.g., for nucleic acid sequencing) may use any polymerase that is capable of synthesizing a nucleic acid complementary to a target nucleic acid molecule. In some embodiments, a polymerase may be a DNA polymerase, an RNA polymerase, a reverse transcriptase, and/or a mutant or altered form of one or more thereof.

Examples of polymerases include, but are not limited to, a DNA polymerase, an RNA polymerase, a thermostable polymerase, a wild-type polymerase, a modified polymerase, E. coli DNA polymerase I, T7 DNA polymerase, bacteriophage T4 DNA polymerase φ29 (psi29) DNA polymerase, Taq polymerase, Tth polymerase, Tli polymerase, Pfu polymerase, Pwo polymerase, VENT polymerase, DEEPVENT polymerase, EX-Taq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tca polymerase, Tih polymerase, Tfi polymerase, Platinum Taq polymerases, Tbr polymerase, Tfl polymerase, Tth polymerase, Pfutubo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof. In some embodiments, the polymerase is a single subunit polymerase. Non-limiting examples of DNA polymerases and their properties are described in detail in, among other places, DNA Replication 2nd edition, Kornberg and Baker, W. H. Freeman, New York, N.Y. (1991).

Upon base pairing between a nucleobase of a target nucleic acid and the complementary dNTP, the polymerase incorporates the dNTP into the newly synthesized nucleic acid strand by forming a phosphodiester bond between the 3′ hydroxyl end of the newly synthesized strand and the alpha phosphate of the dNTP. In examples in which the luminescent tag conjugated to the dNTP is a fluorophore, its presence is signaled by excitation and a pulse of emission is detected during or after the step of incorporation. For detection labels that are conjugated to the terminal (gamma) phosphate of the dNTP, incorporation of the dNTP into the newly synthesized strand results in release the beta and gamma phosphates and the detection label, which is free to diffuse in the sample well, resulting in a decrease in emission detected from the fluorophore.

In some embodiments, the polymerase is a polymerase with high processivity. However, in some embodiments, the polymerase is a polymerase with reduced processivity. Polymerase processivity generally refers to the capability of a polymerase to consecutively incorporate dNTPs into a nucleic acid template without releasing the nucleic acid template. In some embodiments, the polymerase is a polymerase with low 5′-3′ exonuclease activity and/or 3′-5′ exonuclease. In some embodiments, the polymerase is modified (e.g., by amino acid substitution) to have reduced 5′-3′ exonuclease activity and/or 3′-5′ activity relative to a corresponding wild-type polymerase. Further non-limiting examples of DNA polymerases include 9° Nm™ DNA polymerase (New England Biolabs), and a P680G mutant of the Klenow exopolymerase (Tuske et al. (2000) JBC 275(31):23759-23768). In some embodiments, a polymerase having reduced processivity provides increased accuracy for sequencing templates containing one or more stretches of nucleotide repeats (e.g., two or more sequential bases of the same type). In some embodiments, the polymerase is a polymerase that has a higher affinity for a labeled nucleotide than for a non-labeled nucleic acid.

In other aspects, the disclosure provides methods of sequencing target nucleic acids by sequencing a plurality of nucleic acid fragments, wherein the target nucleic acid comprises the fragments. In certain embodiments, the method comprises combining a plurality of fragment sequences to provide a sequence or partial sequence for the parent target nucleic acid. In some embodiments, the step of combining is performed by computer hardware and software. The methods described herein may allow for a set of related target nucleic acids, such as an entire chromosome or genome to be sequenced.

During sequencing, a polymerizing enzyme may couple (e.g., attach) to a priming location of a target nucleic acid molecule. The priming location can be a primer that is complementary to a portion of the target nucleic acid molecule. As an alternative the priming location is a gap or nick that is provided within a double stranded segment of the target nucleic acid molecule. A gap or nick can be from 0 to at least 1, 2, 3, 4, 5, 6, 7, 8, 9, 10, 20, 30, or 40 nucleotides in length. A nick can provide a break in one strand of a double stranded sequence, which can provide a priming location for a polymerizing enzyme, such as, for example, a strand displacing polymerase enzyme.

In some cases, a sequencing primer can be annealed to a target nucleic acid molecule that may or may not be immobilized to a solid support. A solid support can comprise, for example, a sample well (e.g., a nanoaperture, a reaction chamber) on a microchip (or chip) used for nucleic acid sequencing. In some embodiments, a sequencing primer may be immobilized to a solid support and hybridization of the target nucleic acid molecule also immobilizes the target nucleic acid molecule to the solid support. In some embodiments, a polymerase is immobilized to a solid support and soluble primer and target nucleic acid are contacted to the polymerase. However, in some embodiments a complex comprising a polymerase, a target nucleic acid and a primer is formed in solution and the complex is immobilized to a solid support (e.g., via immobilization of the polymerase, primer, and/or target nucleic acid).

Under appropriate conditions, a polymerase enzyme that is contacted to an annealed primer/target nucleic acid can add or incorporate one or more nucleotides onto the primer, and nucleotides can be added to the primer in a 5′ to 3′, template-dependent fashion. Such incorporation of nucleotides onto a primer (e.g., via the action of a polymerase) can generally be referred to as a primer extension reaction. Each nucleotide can be associated with a detectable tag that can be detected and identified (e.g., based on its luminescent lifetime and/or other characteristics) during the nucleic acid extension reaction and used to determine each nucleotide incorporated into the extended primer and, thus, a sequence of the newly synthesized nucleic acid molecule. Via sequence complementarity of the newly synthesized nucleic acid molecule, the sequence of the target nucleic acid molecule can also be determined. In some cases, annealing of a sequencing primer to a target nucleic acid molecule and incorporation of nucleotides to the sequencing primer can occur at similar reaction conditions (e.g., the same or similar reaction temperature) or at differing reaction conditions (e.g., different reaction temperatures).

Accordingly, in some aspects, the disclosure provides methods and compositions for determining nucleotide sequence information from nucleic acid biomolecules (e.g., for sequencing a polynucleotide). In some embodiments, nucleotide sequence information can be determined for single nucleic acid biomolecules. The sequencing methods of the disclosure may comprise “sequencing by synthesis” assays. The sequencing methods of the disclosure may comprise time-course (or time domain) measurement assays. In some embodiments, sequencing by synthesis methods can include the presence of a population of target nucleic acid molecules (e.g., copies of a target nucleic acid) and/or a step of amplification of the target nucleic acid to achieve a population of target nucleic acids. However, in some embodiments sequencing by synthesis is used to determine the sequence of a single molecule in each reaction that is being evaluated (and nucleic acid amplification is not required to prepare the target template for sequencing). In some embodiments, a plurality of single molecule sequencing reactions are performed in parallel (e.g., on a single chip) according to aspects of the present application. For example, in some embodiments, a plurality of single molecule sequencing reactions are each performed in separate reaction chambers (e.g., nanoapertures, sample wells) that have been fabricated onto a single chip.

In some embodiments, the disclosed nucleic acid sequencing methods are performed in sample wells that have been fabricated on a complementary metal oxide semiconductor (CMOS) chip. Each well may be aligned over a CMOS photodiode. In some embodiments, the disclosed nucleic acid sequencing methods are performed on a CMOS chip in conjunction with benchtop integrated device. In some embodiments, the sequencing methods are performed on a CMOS chip in conjunction with a one- or multiple-channel photodetection sequencing-by-synthesis integrated device (see FIG. 2A).

In some embodiments, the nucleic acid of the luminescently labeled nucleotide further comprises a third oligonucleotide strand annealed to at least one of the first and second oligonucleotide strands. In some embodiments, the nucleic acid further comprises a fourth oligonucleotide strand annealed to at least one of the first, second, and third oligonucleotide strands. In some embodiments, the oligonucleotide strands form a Holliday junction.

In some aspects, the disclosure provides methods of determining the sequence of a template nucleic acid. In some embodiments, the methods include a step comprising exposing a complex in a target volume, the complex comprising the template nucleic acid, a primer, and a polymerizing enzyme, to a plurality of types of luminescently labeled nucleotides. In some embodiments, each type of luminescently labeled nucleotide comprises one or more luminescent labels (e.g., comprise identical luminescent labels or comprise different luminescent labels) connected to one or more nucleoside polyphosphates via a nucleic acid. In some embodiments, the nucleic acid comprises a protecting molecule. Accordingly, in some aspects, the disclosure provides methods of nucleic acid sequencing that utilize any of the luminescently labeled nucleoside polyphosphate compositions described herein.

In some embodiments, the methods further comprise a step of directing a series of pulses of one or more excitation energies towards a vicinity of the target volume. In some embodiments, the methods further comprise a step of detecting a plurality of emitted photons from luminescently labeled nucleotides during sequential incorporation into a nucleic acid comprising the primer. In some embodiments, the methods further comprise a step of identifying the sequence of incorporated nucleotides by determining timing and optionally frequency of the emitted photons.

In some embodiments, four different types of nucleotides (e.g., adenine, guanine, cytosine, thymine/uracil) in a reaction mixture can each be labeled with one or more luminescent molecules (e.g., one or more luminescent labels). In some embodiments, each type of nucleotide can be connected to more than one of the same luminescent molecule (e.g., two or more of the same fluorescent dye connected to a nucleotide). In some embodiments, each luminescent molecule can be connected to more than one nucleotide (e.g., two or more of the same nucleotide). In some embodiments, more than one nucleotide can be connected (e.g., via a nucleic acid linker comprising one or more protecting molecules) to more than one luminescent molecule. In some embodiments, all four nucleotides are labeled with luminescent molecules that absorb and emit within the same spectral range (e.g., 520-570 nm).

Embodiments are capable of sequencing single nucleic acid molecules with high accuracy and long read lengths, such as an accuracy of at least about 50%, 60%, 70%, 75%, 80%, 85%, 90%, 95%, 96%, 97%, 98%, 99%, 99.9%, 99.99%, 99.999%, or 99.9999%, and/or read lengths greater than or equal to about 10 base pairs (bp), 50 bp, 100 bp, 200 bp, 300 bp, 400 bp, 500 bp, 1000 bp, 10,000 bp, 20,000 bp, 30,000 bp, 40,000 bp, 50,000 bp, or 100,000 bp. In some embodiments, the target nucleic acid molecule used in single molecule sequencing is a single stranded target nucleic acid (e.g., deoxyribonucleic acid (DNA), DNA derivatives, ribonucleic acid (RNA), RNA derivatives) template that is added or immobilized to a sample well (e.g., nanoaperture) containing at least one additional component of a sequencing reaction (e.g., a polymerase such as, a DNA polymerase, a sequencing primer) immobilized or attached to a solid support such as the bottom or side walls of the sample well. The target nucleic acid molecule or the polymerase can be attached to a sample wall, such as at the bottom or side walls of the sample well directly or through a linker. The sample well (e.g., nanoaperture) also can contain any other reagents needed for nucleic acid synthesis via a primer extension reaction, such as, for example suitable buffers, co-factors, enzymes (e.g., a polymerase) and deoxyribonucleoside polyphosphates, such as, e.g., deoxyribonucleoside triphosphates, including deoxyadenosine triphosphate (dATP), deoxycytidine triphosphate (dCTP), deoxyguanosine triphosphate (dGTP), deoxyuridine triphosphate (dUTP) and deoxythymidine triphosphate (dTTP) dNTPs, that include luminescent tags, such as fluorophores. In some embodiments, each class of dNTPs (e.g., adenine-containing dNTPs (e.g., dATP), cytosine-containing dNTPs (e.g., dCTP), guanine-containing dNTPs (e.g., dGTP), uracil-containing dNTPs (e.g., dUTPs) and thymine-containing dNTPs (e.g., dTTP)) is conjugated to a distinct luminescent tag such that detection of light emitted from the tag indicates the identity of the dNTP that was incorporated into the newly synthesized nucleic acid. Emitted light from the luminescent tag can be detected and attributed to its appropriate luminescent tag (and, thus, associated dNTP) via any suitable device and/or method, including such devices and methods for detection described elsewhere herein. The luminescent tag may be conjugated to the dNTP at any position such that the presence of the luminescent tag does not inhibit the incorporation of the dNTP into the newly synthesized nucleic acid strand or the activity of the polymerase. In some embodiments, the luminescent tag is conjugated to the terminal phosphate (e.g., the gamma phosphate) of the dNTP.

In some embodiments, the single-stranded target nucleic acid template can be contacted with a sequencing primer, dNTPs, polymerase and other reagents necessary for nucleic acid synthesis. In some embodiments, all appropriate dNTPs can be contacted with the single-stranded target nucleic acid template simultaneously (e.g., all dNTPs are simultaneously present) such that incorporation of dNTPs can occur continuously. In other embodiments, the dNTPs can be contacted with the single-stranded target nucleic acid template sequentially, where the single-stranded target nucleic acid template is contacted with each appropriate dNTP separately, with washing steps in between contact of the single-stranded target nucleic acid template with differing dNTPs. Such a cycle of contacting the single-stranded target nucleic acid template with each dNTP separately followed by washing can be repeated for each successive base position of the single-stranded target nucleic acid template to be identified.

In some embodiments, the sequencing primer anneals to the single-stranded target nucleic acid template and the polymerase consecutively incorporates the dNTPs (or other deoxyribonucleoside polyphosphate) to the primer based on the single-stranded target nucleic acid template. The unique luminescent tag associated with each incorporated dNTP can be excited with the appropriate excitation light during or after incorporation of the dNTP to the primer and its emission can be subsequently detected, using, any suitable device(s) and/or method(s), including devices and methods for detection described elsewhere herein. Detection of a particular emission of light (e.g., having a particular emission (luminescent) lifetime, intensity, spectrum and/or combination thereof) can be attributed to a particular dNTP incorporated. The sequence obtained from the collection of detected luminescent tags can then be used to determine the sequence of the single-stranded target nucleic acid template via sequence complementarity.

While the present disclosure makes reference to dNTPs, devices, systems and methods provided herein may be used with various types of nucleotides, such as ribonucleotides and deoxyribonucleotides (e.g., deoxyribonucleoside polyphosphates with at least 4, 5, 6, 7, 8, 9, or 10 phosphate groups). Such ribonucleotides and deoxyribonucleotides can include various types of tags (or markers) and linkers. In some embodiments, the present disclosure provides methods and compositions that may be advantageously utilized in the technologies described in U.S. patent application Ser. Nos. 14/543,865, 14/543,867, 14/543,888, 14/821,656, 14/821,686, 14/821,688, 15/161,067, 15/161,088, 15/161,125, 15/255,245, 15/255,303, 15/255,624, 15/261,697, and 15/261,724, the contents of each of which is incorporated herein by reference.

Signals emitted upon the incorporation of nucleotides can be stored in memory and processed at a later point in time to determine the sequence of the target nucleic acid template. This may include comparing the signals to a reference signals to determine the identities of the incorporated nucleotides as a function of time. Alternatively or in addition to, signal emitted upon the incorporation of nucleotide can be collected and processed in real time (e.g., upon nucleotide incorporation) to determine the sequence of the target nucleic acid template in real time.

The term “nucleic acid,” as used herein, generally refers to a molecule comprising one or more nucleic acid subunits. A nucleic acid may include one or more subunits selected from adenine (A), cytosine (C), guanine (G), thymine (T), and uracil (U), or variants thereof. In some examples, a nucleic acid is deoxyribonucleic acid (DNA) or ribonucleic acid (RNA), or derivatives thereof. A nucleic acid may be single-stranded or double stranded. A nucleic acid may be circular.

The term “nucleotide,” as used herein, generally refers to a nucleic acid subunit, which can include A, C, G, T or U, or variants or analogs thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (e.g., A or G, or variant or analogs thereof) or a pyrimidine (e.g., C, T or U, or variant or analogs thereof). A subunit can enable individual nucleic acid bases or groups of bases (e.g., AA, TA, AT, GC, CG, CT, TC, GT, TG, AC, CA, or uracil-counterparts thereof) to be resolved.

A nucleotide generally includes a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Deoxyribonucleotides are nucleotides in which the sugar is deoxyribose. A nucleotide can be a nucleoside monophosphate or a nucleoside polyphosphate. A nucleotide can be a deoxyribonucleoside polyphosphate, such as, e.g., a deoxyribonucleoside triphosphate, which can be selected from deoxyadenosine triphosphate (dATP), deoxycytidine triphosphate (dCTP), deoxyguanosine triphosphate (dGTP), deoxyuridine triphosphate (dUTP) and deoxythymidine triphosphate (dTTP) dNTPs, that include detectable tags, such as luminescent tags or markers (e.g., fluorophores).

A nucleoside polyphosphate can have ‘n’ phosphate groups, where ‘n’ is a number that is greater than or equal to 2, 3, 4, 5, 6, 7, 8, 9, or 10. Examples of nucleoside polyphosphates include nucleoside diphosphate and nucleoside triphosphate. A nucleotide can be a terminal phosphate labeled nucleoside, such as a terminal phosphate labeled nucleoside polyphosphate. Such label can be a luminescent (e.g., fluorescent or chemiluminescent) label, a fluorogenic label, a colored label, a chromogenic label, a mass tag, an electrostatic label, or an electrochemical label. A label (or marker) can be coupled to a terminal phosphate through a linker. The linker can include, for example, at least one or a plurality of hydroxyl groups, sulfhydryl groups, amino groups or haloalkyl groups, which may be suitable for forming, for example, a phosphate ester, a thioester, a phosphoramidate or an alkyl phosphonate linkage at the terminal phosphate of a natural or modified nucleotide. A linker can be cleavable so as to separate a label from the terminal phosphate, such as with the aid of a polymerization enzyme. Examples of nucleotides and linkers are provided in U.S. Pat. No. 7,041,812, which is incorporated herein by reference.

A nucleotide (e.g., a nucleotide polyphosphate) can comprise a methylated nucleobase. For example, a methylated nucleotide can be a nucleotide that comprises one or more methyl groups attached to the nucleobase (e.g., attached directly to a ring of the nucleobase, attached to a substituent of a ring of the nucleobase). Exemplary methylated nucleobases include 1-methylthymine, 1-methyluracil, 3-methyluracil, 3-methylcytosine, 5-methylcytosine, 1-methyladenine, 2-methyladenine, 7-methyladenine, N6-methyladenine, N6,N6-dimethyladenine, 1-methylguanine, 7-methylguanine, N2-methylguanine, and N2,N2-dimethylguanine.

The term “primer,” as used herein, generally refers to a nucleic acid molecule (e.g., an oligonucleotide), which can include a sequence comprising A, C, G, T and/or U, or variants or analogs thereof. A primer can be a synthetic oligonucleotide comprising DNA, RNA, PNA, or variants or analogs thereof. A primer can be designed such that its nucleotide sequence is complementary to a target strand, or the primer can comprise a random nucleotide sequence. In some embodiments, a primer can comprise a tail (e.g., a poly-A tail, an index adaptor, a molecular barcode, etc.). In some embodiments, a primer can comprise 5 to 15 bases, 10 to 20 bases, 15 to 25 bases, 20 to 30 bases, 25 to 35 bases, 30 to 40 bases, 35 to 45 bases, 40 to 50 bases, 45 to 55 bases, 50 to 60 bases, 55 to 65 bases, 60 to 70 bases, 65 to 75 bases, 70 to 80 bases, 75 to 85 bases, 80 to 90 bases, 85 to 95 bases, 90 to 100 bases, 95 to 105 bases, 100 to 150 bases, 125 to 175 bases, 150 to 200 bases, or more than 200 bases.

Peptide Sequencing

In some embodiments, aspects of the disclosure can be used to assay biological samples, for example to determine the identity or sequence of one or more polypeptides in the sample and/or to determine the presence or absence of one or more variants (e.g., one or more amino acid substitutions in a polypeptide of interest) in the sample. In some embodiments, the compounds described herein may be subjected to peptide sequencing (also referred to as “polypeptide sequencing”) by detecting single molecule binding interactions during a peptide degradation process. In some embodiments, the peptide of interest is covalently or non-covalently attached to a surface.

As used herein, sequencing a polypeptide refers to determining sequence information for a polypeptide. In some embodiments, this can involve determining the identity of each sequential amino acid for a portion (or all) of the polypeptide. However, in some embodiments, this can involve assessing the identity of a subset of amino acids within the polypeptide (and, for instance, determining the relative position of one or more amino acid types without determining the identity of each amino acid in the polypeptide). In some embodiments, amino acid content information can be obtained from a polypeptide without directly determining the relative position of different types of amino acids in the polypeptide. The amino acid content alone may be used to infer the identity of the polypeptide that is present (e.g., by comparing the amino acid content to a database of polypeptide information and determining which polypeptide(s) have the same amino acid content).

In some aspects, the methods for sequencing of a peptide provided herein may be performed by identifying one or more types of amino acids of a polypeptide. In some embodiments, one or more amino acids (e.g., terminal amino acids and/or internal amino acids) of the polypeptide are labeled (e.g., directly or indirectly, for example using a binding agent such as an amino acid recognition molecule) and the relative positions of the labeled amino acids in the polypeptide are determined. In some embodiments, the relative positions of amino acids in a polypeptide are determined using a series of amino acid labeling. In certain embodiments, the relative positions of amino acids in a polypeptide are determined using a series of labeling and cleavage steps. However, in some embodiments, the relative position of labeled amino acids in a polypeptide can be determined without removing amino acids from the polypeptide but by translocating a labeled polypeptide through a pore (e.g., a protein channel) and detecting a signal (e.g., a FRET signal) from the labeled amino acid(s) during translocation through the pore in order to determine the relative position of the labeled amino acids in the polypeptide molecule.

In some embodiments, the identity of a terminal amino acid (e.g., an N-terminal or a C-terminal amino acid) is assessed after which the terminal amino acid is removed and the identity of the next amino acid at the terminus is assessed, and this process is repeated until a plurality of successive amino acids in the polypeptide are assessed. In some embodiments, assessing the identity of an amino acid comprises determining the type of amino acid that is present. In some embodiments, determining the type of amino acid comprises determining the actual amino acid identity, for example by determining which of the naturally-occurring 20 amino acids is the terminal amino acid is (e.g., using a binding agent that is specific for an individual terminal amino acid). In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

However, in some embodiments assessing the identity of a terminal amino acid type can comprise determining a subset of potential amino acids that can be present at the terminus of the polypeptide. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be at the terminus of the polypeptide (e.g., using a binding agent that binds to a specified subset of two or more terminal amino acids). In some embodiments, assessing the identity of a terminal amino acid type comprises determining that an amino acid comprises a post-translational modification.

In some embodiments, methods provided herein comprise contacting a polypeptide with a labeled amino acid recognition molecule that selectively binds one type of terminal amino acid. In some embodiments, a labeled recognition molecule selectively binds one type of terminal amino acid over other types of terminal amino acids. In some embodiments, a labeled recognition molecule selectively binds one type of terminal amino acid over an internal amino acid of the same type. In yet other embodiments, a labeled recognition molecule selectively binds one type of amino acid at any position of a polypeptide, e.g., the same type of amino acid as a terminal amino acid and an internal amino acid.

In some embodiments, polypeptide sequencing can proceed by contacting the polypeptide with one or more amino acid recognition molecules that associate with one or more types of terminal amino acids. In some embodiments, a labeled amino acid recognition molecule interacts with polypeptide by associating with the terminal amino acid.

In some embodiments, one or more types of amino acids are identified by detecting one or more electrical characteristics of a labeled recognition molecule. In some embodiments, a labeled recognition molecule comprises a recognition molecule that selectively binds one type of amino acid and a conductivity label that is associated with the recognition molecule. In this way, the one or more electrical characteristics (e.g., charge, current oscillation color, and other electrical characteristics) may be associated with the selective binding of the recognition molecule to identify an amino acid of a polypeptide. In some embodiments, a plurality of types of labeled recognition molecules may be used in a method according to the application, wherein each type comprises a conductivity label that produces a change in an electrical signal (e.g., a change in conductance, such as a change in amplitude of conductivity and conductivity transitions of a characteristic pattern) that is uniquely identifiable from among the plurality. In some embodiments, the plurality of types of labeled recognition molecules each comprises a conductivity label having a different number of charged groups (e.g., a different number of negatively and/or positively charged groups). Accordingly, in some embodiments, a conductivity label is a charge label. Examples of charge labels include dendrimers, nanoparticles, nucleic acids and other polymers having multiple charged groups. In some embodiments, a conductivity label is uniquely identifiable by its net charge (e.g., a net positive charge or a net negative charge), by its charge density, and/or by its number of charged groups.

Accordingly, in some embodiments, the one or more types of amino acids are identified by detecting luminescent lifetime or luminescent intensity of one or more labeled amino acid recognition molecules that selectively bind the one or more types of amino acids. In some embodiments, methods provided herein comprise contacting a polypeptide with one or more labeled amino acid recognition molecules (or recognition molecules) that selectively bind one or more types of terminal amino acids. As an illustrative and non-limiting example, where four labeled recognition molecules are used in a method of the application, any one reagent selectively binds one type of terminal amino acid that is different from another type of amino acid to which any of the other three selectively binds (e.g., a first reagent binds a first type, a second reagent binds a second type, a third reagent binds a third type, and a fourth reagent binds a fourth type of terminal amino acid).

In some embodiments, a recognition molecule may be engineered by one skilled in the art using conventionally known techniques. In some embodiments, desirable properties may include an ability to bind selectively and with high affinity to one type of amino acid only when it is located at a terminus (e.g., an N-terminus or a C-terminus) of a polypeptide. In yet other embodiments, desirable properties may include an ability to bind selectively and with high affinity to one type of amino acid when it is located at a terminus (e.g., an N-terminus or a C-terminus) of a polypeptide and when it is located at an internal position of the polypeptide. In some embodiments, desirable properties include an ability to bind selectively and with low affinity (e.g., with a K_{D} of about 50 nM or higher, for example, between about 50 nM and about 50 μM, between about 100 nM and about 10 μM, between about 500 nM and about 50 μM) to more than one type of amino acid. For example, in some aspects, the disclosure provides methods of sequencing by detecting reversible binding interactions. Advantageously, such methods may be performed using a recognition molecule that reversibly binds with low affinity to more than one type of amino acid (e.g., a subset of amino acid types).

In some embodiments, polypeptide sequencing comprises providing a polypeptide of interest that is coated to a surface of a solid support (e.g., attached to a bottom surface of a sample well) through a coupling moiety and/or a coating molecule. In some embodiments, the coupling moiety-coating molecule conjugate is formed by a linkage between a biotin coupling moiety and a streptavidin coupling moiety coated to the surface of a well.

In some embodiments, the polypeptide of interest is immobilized to the surface through a coupling moiety at one terminal end such that the other terminal end is free for detecting and cleaving of a terminal amino acid in a sequencing reaction. Accordingly, in some embodiments, the reagents used in certain polypeptide sequencing reactions preferentially interact with terminal amino acids at the non-immobilized (e.g., free) terminus of the polypeptide. In this way, the polypeptide remains immobilized over repeated cycles of detecting and cleaving.

In some embodiments, polypeptide sequencing can proceed by contacting the polypeptide with one or more amino acid recognition molecules that associate with one or more types of terminal amino acids. In some embodiments, a labeled amino acid recognition molecule interacts with polypeptide by associating with the terminal amino acid.

In some embodiments, the method further comprises identifying the amino acid (terminal or internal amino acid) of the polypeptide by detecting the labeled amino acid recognition molecule. In some embodiments, detecting comprises detecting a luminescence from labeled amino acid recognition molecule. In some embodiments, the luminescence is uniquely associated with the labeled amino acid recognition molecule, and the luminescence is thereby associated with the type of amino acid to which the labeled amino acid recognition molecule selectively binds. As such, in some embodiments, the type of amino acid is identified by determining one or more luminescence properties of the labeled amino acid recognition molecule.

In other embodiments, polypeptide sequencing proceeds by removing the terminal amino acid by contacting the polypeptide with an exopeptidase that binds and cleaves the terminal amino acid of the polypeptide. Upon removal of the terminal amino acid by exopeptidase, polypeptide sequencing proceeds by subjecting the polypeptide (having n-1 amino acids) to additional cycles of terminal amino acid recognition and cleavage. In some embodiments, these steps may occur in the same reaction mixture, e.g., as in a dynamic peptide sequencing reaction. In some embodiments, these steps may be carried out using other methods known in the art, such as peptide sequencing by Edman degradation.

In some embodiments, dynamic polypeptide sequencing is carried out in real-time by evaluating binding interactions of terminal amino acids with labeled amino acid recognition molecules, and optionally a cleaving reagent (e.g., an exopeptidase). A labeled amino acid recognition molecule may associate with (e.g., bind to) and dissociate from a terminal amino acid, which gives rise to a series of pulses in signal output which may be used to identify the terminal amino acid. In some embodiments, the series of pulses provide a pulsing pattern (e.g., a characteristic pattern) which may be diagnostic of the identity of the corresponding terminal amino acid.

Edman degradation involves repeated cycles of modifying and cleaving the terminal amino acid of a polypeptide, wherein each successively cleaved amino acid is identified to determine an amino acid sequence of the polypeptide. Peptide sequencing by conventional Edman degradation can be carried out by (1) contacting a polypeptide of interest with one or more amino acid recognition molecules that selectively bind one or more types of terminal amino acids. In some embodiments, step (1) further comprises removing any of the one or more labeled amino acid recognition molecules that do not selectively bind the polypeptide.

In some embodiments, dynamic peptide sequencing is performed by observing different association events, e.g., association events between an amino acid recognition molecule and an amino acid at a terminal end of a peptide, wherein each association event produces a change in magnitude of a signal, e.g., a luminescent signal, that persists for a duration of time.

In some embodiments, observing different association events, e.g., association events between an amino acid recognition molecule and an amino acid at a terminal end of a peptide, can be performed during a peptide degradation process. In some embodiments, a transition from one characteristic signal pattern to another is indicative of amino acid cleavage (e.g., amino acid cleavage resulting from peptide degradation). In some embodiments, amino acid cleavage refers to the removal of at least one amino acid from a terminus of a polypeptide (e.g., the removal of at least one terminal amino acid from the polypeptide). In some embodiments, amino acid cleavage is determined by inference based on a time duration between characteristic signal patterns. In some embodiments, amino acid cleavage is determined by detecting a change in signal produced by association of a labeled cleaving reagent with an amino acid at the terminus of the polypeptide. In some embodiments, wherein a cleaving agent (exopeptidase) is used, as amino acids are sequentially cleaved from the terminus of the polypeptide during degradation, a series of changes in magnitude, or a series of signal pulses, is detected.

In some embodiments, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, different characteristic patterns corresponding to different types of amino acids in a single polypeptide may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). It should be appreciated that, in some embodiments, smaller differences in mean pulse duration between different characteristic patterns may require a greater number of pulse durations within each characteristic pattern to distinguish one from another with statistical confidence.

In some aspects, the disclosure provides methods of immobilizing a peptide, or an amino acid recognition molecule, to a surface by attaching any one of the compounds described herein to a surface of a sample well. In some embodiments, the surface is functionalized with a coupling moiety configured for attachment (e.g., covalent attachment) to a functionalized terminal end of a peptide. In some embodiments, the methods comprise immobilizing a single peptide to a surface of each of a plurality of sample wells. In some embodiments, confining a single peptide per sample well is advantageous for single molecule detection methods, e.g., single molecule peptide sequencing.

In some embodiments, peptide comprising functionalized terminal end is contacted with complementary coupling moiety. In some embodiments, functionalized terminal end and coupling moiety comprise non-covalent binding partners, e.g., which form a non-covalent linkage between peptide and the sample well surface. Examples of non-covalent binding partners include protein-protein binding partners (e.g., barnase and barstar), and protein-ligand binding partners (e.g., biotin and streptavidin).

Sequencing Using Luminescent Lifetime Measurements

Individual pixels on an integrated photodetector may be capable of luminescent lifetime measurements used to identify fluorophores and/or reporters that label one or more targets, such as molecules or specific locations on molecules. Although the following description discusses sample identification based on lifetime measurement, it should be understood that one or more additional or alternative characteristics of a signal emitted from a reaction chamber in response to excitation light may be used to identify a sample. For example, any of the techniques described in U.S. patent application Ser. No. 17/190,331 titled “INTEGRATED SENSOR FOR MULTI-DIMENSIONAL SIGNAL ANALYSIS,” filed Mar. 2, 2021 under Attorney Docket No. R0708.70090US01 which is hereby incorporated by reference in its entirety herein may be used.

Any one or more molecules of interest may be labeled with a fluorophore, including proteins, amino acids, enzymes, lipids, nucleotides, DNA, and RNA. When combined with detecting spectra of the emitted light or other labeling techniques, luminescent lifetime may increase the total number of fluorophores and/or reporters that can be used. Identification based on lifetime may be used for single molecule analytical methods to provide information about characteristics of molecular interactions in complex mixtures that may include protein-protein interactions, enzymatic activity, molecular dynamics, and/or diffusion on membranes. Additionally, fluorophores with different luminescent lifetimes may be used to tag target components in various assay methods that are based on presence of a labeled component. In some embodiments, components may be separated, such as by using microfluidic systems, based on detecting particular lifetimes of fluorophores.

Measuring luminescent lifetimes may be used in combination with other analytical methods. For an example, luminescent lifetimes may be used in combination with fluorescence resonance energy transfer (FRET) techniques to discriminate between the states and/or environments of donor and acceptor fluorophores located on one or more molecules. Such measurements may be used to determine the distance between the donor and the acceptor. In some instances, energy transfer from the donor to the acceptor may decrease the lifetime of the donor. In another example, luminescent lifetime measurements may be used in DNA sequencing applications, such as where four fluorophores having different lifetimes may be used to label the four different nucleotides (A, T, G, C) in a DNA molecule with an unknown sequence of nucleotides. The luminescent lifetimes, instead of emission spectra, of the fluorophores may be used to identify the sequence of nucleotides. By using luminescent lifetime instead of emission spectra for certain techniques, accuracy and measurement resolution may increase because artifacts due to absolute intensity measurements are reduced. Additionally, lifetime measurements may reduce the complexity and/or expense of the system because fewer excitation energy wavelengths are required and/or fewer emission energy wavelengths need be detected.

In some embodiments, an integrated photodetector as described in the present disclosure can measure or discriminate luminescent lifetimes. Luminescent lifetime measurements are based on exciting one or more fluorescent molecules, and measuring the time variation in the emitted luminescence. The probability of a fluorescent molecule to emit a photon after the fluorescent molecule reaches an excited state decreases exponentially over time. The rate at which the probability decreases may be characteristic of a fluorescent molecule, and may be different for different fluorescent molecules. Detecting the temporal characteristics of light emitted by fluorescent molecules may allow identifying fluorescent molecules and/or discriminating fluorescent molecules with respect to one another. Luminescent molecules are also referred to herein as luminescent markers (or simply “markers”), labels and fluorophores.

After reaching an excited state, a marker may emit a photon with a certain probability at a given time. The probability of a photon being emitted from an excited marker may decrease over time after excitation of the marker. The decrease in the probability of a photon being emitted over time may be represented by an exponential decay function p(t)=e^{-t/τ}, where p(t) is the probability of photon emission at a time, t, and τ is a temporal parameter of the marker. The temporal parameter τ indicates a time after excitation when the probability of the marker emitting a photon is a certain value. The temporal parameter, τ, is a property of a marker that may be distinct from its absorption and emission spectral properties. Such a temporal parameter, τ, is referred to as the luminescent lifetime, or simply the “lifetime”, of a marker. Markers may have luminescent lifetimes ranging from 0.1-20 ns, in some embodiments. However, the techniques described herein are not limited as to the lifetimes of the marker(s) used.

The lifetime of a marker may be used to distinguish among more than one marker, and/or may be used to identify marker(s). In some embodiments, luminescent lifetime measurements may be performed in which a plurality of markers having different lifetimes are excited by an excitation source. As an example, four markers having lifetimes of 0.5, 1, 2, and 3 nanoseconds, respectively, may be excited by a light source that emits light having a selected wavelength (e.g., 635 nm, by way of example). The markers may be identified or differentiated from each other based on measuring the lifetime of the light emitted by the markers.

Luminescent lifetime measurements may use relative intensity measurements by comparing how intensity changes over time, as opposed to absolute intensity values. As a result, luminescent lifetime measurements may avoid some of the difficulties of absolute intensity measurements. Absolute intensity measurements may depend on the concentration of fluorophores present and calibration steps may be needed for varying fluorophore concentrations. By contrast, luminescent lifetime measurements may be insensitive to the concentration of fluorophores.

Differentiating between markers by lifetime measurements may allow for fewer wavelengths of excitation light to be used than when the markers are differentiated by measurements of emission spectra. In some embodiments, sensors, filters, and/or diffractive optics may be reduced in number or eliminated when using fewer wavelengths of excitation light and/or luminescent light. In some embodiments, labeling may be performed with markers that have different lifetimes, and the markers may be excited by light having the same excitation wavelength or spectrum. In some embodiments, an excitation light source may be used that emits light of a single wavelength or spectrum, which may reduce the cost. However, the techniques described herein are not limited in this respect, as any number of excitation light wavelengths or spectra may be used. In some embodiments, an integrated photodetector may be used to determine both spectral and temporal information regarding received light. In some embodiments a quantitative analysis of the types of molecule(s) present may be performed by determining a temporal parameter, a spectral parameter, or a combination of the temporal and spectral parameters of the emitted luminescence from a marker.

An integrated device having an integrated photodetector according to aspects of the present application may be designed with suitable functions for a variety of detection and imaging applications. Such an integrated photodetector can have the ability to detect light within one or more time intervals, or “time bins.” To collect information regarding the time of arrival of the light, charge carriers are generated in response to incident photons and can be segregated into respective time bins based upon their time of arrival.

An integrated photodetector that detects the arrival time of incident photons may reduce additional optical filtering (e.g., optical spectral filtering) requirements. As described below, an integrated photodetector according to the present application may include a drain to remove photogenerated carriers at particular times. By removing photogenerated carriers in this manner, unwanted charge carriers produced in response to an excitation light pulse may be discarded without the need for optical filtering to prevent reception of light from the excitation pulse. Such a photodetector may reduce overall design integration complexity, optical and/or filtering components, and/or cost.

In some embodiments, a luminescent lifetime may be determined by measuring the time profile of the emitted luminescence by aggregating collected charge carriers in one or more time bins of the integrated photodetector to detect luminescent intensity values as a function of time. In some embodiments, the lifetime of a marker may be determined by performing multiple measurements where the marker is excited into an excited state and then the time when a photon emits is measured. For each measurement, the excitation source may generate a pulse of excitation light directed to the marker, and the time between the excitation pulse and subsequent photon event from the marker may be determined. Additionally or alternatively, when an excitation pulse occurs repeatedly and periodically, the time between when a photon emission event occurs and the subsequent excitation pulse may be measured, and the measured time may be subtracted from the time interval between excitation pulses (i.e., the period of the excitation pulse waveform) to determine the time of the photon absorption event.

By repeating such experiments with a plurality of excitation pulses, the number of instances a photon is emitted from the marker within a certain time interval after excitation may be determined, which is indicative of the probability of a photon being emitted within such a time interval after excitation. The number of photon emission events collected may be based on the number of excitation pulses emitted to the marker. The number of photon emission events over a measurement period may range from 50-10,000,000 or more, in some embodiments, however, the techniques described herein are not limited in this respect. The number of instances a photon is emitted from the marker within a certain time interval after excitation may populate a histogram representing the number of photon emission events that occur within a series of discrete time intervals or time bins. The number of time bins and/or the time interval of each bin may be set and/or adjusted to identify a particular lifetime and/or a particular marker. The number of time bins and/or the time interval of each bin may depend on the sensor used to detect the photons emitted. The number of time bins may be 1, 2, 3, 4, 5, 6, 7, 8, or more, such as 16, 32, 64, or more. A curve fitting algorithm may be used to fit a curve to the recorded histogram, resulting in a function representing the probability of a photon to be emitted after excitation of the marker at a given time. The above decay function, p(t)=e^{-t/τ}, may be used to approximately fit the histogram data. From such a curve fitting, the temporal parameter or lifetime may be determined. The determined lifetime may be compared to known lifetimes of markers to identify the type of marker present.

A lifetime may be calculated from the intensity values obtained at two time intervals. In some embodiments, the photodetector measures the intensity over at least two time bins. The photons that emit luminescence energy between times t1 and t2 are measured by the photodetector as intensity I1 and luminescence energy emitted between times t3 and t4 are measured as I2. Any suitable number of intensity values may be obtained. Such intensity measurements may then be used to calculate a lifetime. When one fluorophore is present at a time, then the time binned luminescence signal may be fit to a single exponential decay. In some embodiments, only two time bins may be needed to accurately identify the lifetime for a fluorophore. When two or more fluorophores are present, then individual lifetimes may be identified from a combined luminescence signal by fitting the luminescence signal to multiple exponential decays, such as double or triple exponentials. In some embodiments two or more time bins may be needed in order to accurately identify more than one luminescent lifetime from such a signal. However, in some instances with multiple fluorophores, an average luminescent lifetime may be determined by fitting a single exponential decay to the luminescence signal.

In some instances, the probability of a photon emission event and thus the lifetime of a marker may change based on the surroundings and/or conditions of the marker. For example, the lifetime of a marker confined in a volume with a diameter less than the wavelength of the excitation light may be smaller than when the marker is not in the volume. Lifetime measurements with known markers under conditions similar to when the markers are used for labeling may be performed. The lifetimes determined from such measurements with known markers may be used when identifying a marker.

### EXAMPLE 2

An on-chip evaluation of sequencing accuracy following performance of an exemplary method of chip regeneration in an DNA sequencing application according to the disclosure was performed. An array of sample wells on a CMOS chip was loaded with aliquots of a sample containing a known DNA molecule of interest. In this evaluation, three sequential runs of end DNA sequencing performance on aliquots of the sample were performed: the first run was performed on a fresh, clean chip; the second run was performed after applying an exemplary regeneration solution of the disclosure; and the third run was performed after applying the regeneration solution to the chip a second time. As such, the results of the third run represented an evaluation of a second regeneration step. The regeneration solution used in this experiment contained 9 mL HFIP, 0.5 mL 5M ammonium acetate, 1 mL water, biotin-PEG₃ (bis-biotin-PEG₄) at a concentration of between 100 μM and 200 μM.

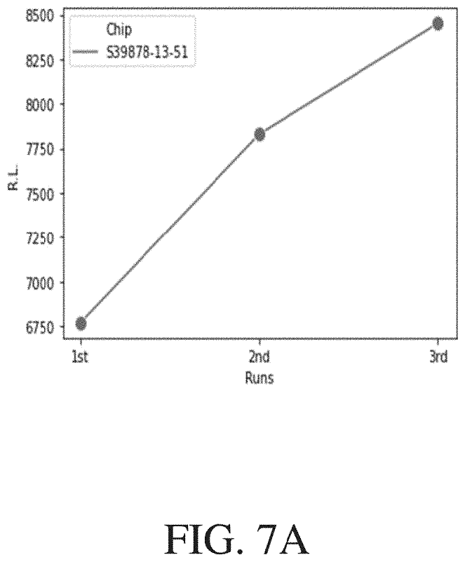

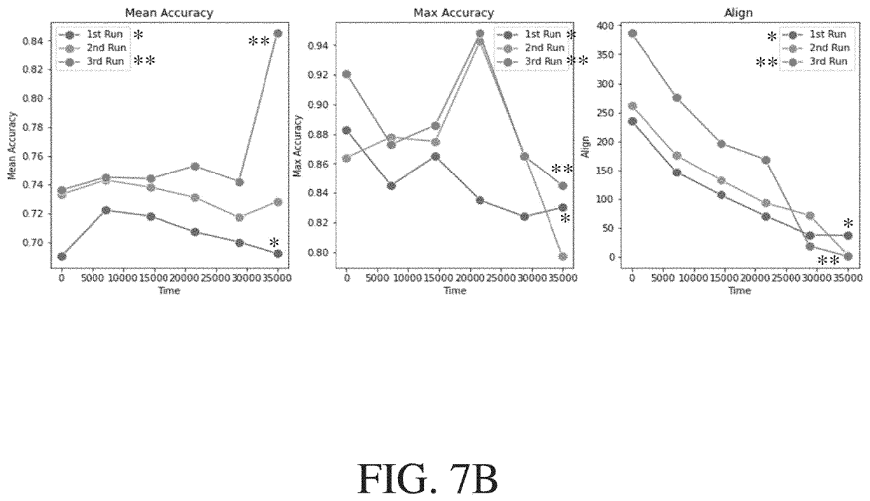

The workup for the regeneration steps of this experiment is as follows: following the first sequencing run, the chip is cleaned of any residues, and then a nucleoside digestion mix (NEB #M0649) was applied to the chip. Then the chip is incubated at 37° C. for 40 minutes. Subsequently, the regeneration solution is applied to the chip, and the chip is further incubated at room temperature for 30 minutes. Lastly, the chip is again cleaned of any residues. Application of the regeneration solution prior to the second and third runs appeared to have successfully dissociated the streptavidin-biotin interactions at the surface of the sample wells. Nucleotide sequencing read length and accuracy for each of the three runs were measured and charted in the graphs shown in FIGS. 7A and 7B. As shown in FIG. 7B, a mean accuracy of ˜74%, and a maximum (or “best”) accuracy of over 94% was observed in the second and third runs. In fact, surprisingly, the second and third runs exhibited comparable to superior sequencing performance to the first run. Thus, in this experiment, chip regeneration and reuse may have improved the readout. FIG. 7A shows mean read lengths among signals acquired from the three runs, which included a read length during the third run of about 8500 bp. FIG. 7B also shows the “alignment score” of each of sequence readout against the known sequence of the molecule.

Subsequently, an on-chip evaluation of sequencing accuracy following performance of an exemplary method of chip regeneration in a peptide sequencing application according to the disclosure was performed. An array of sample wells on a CMOS chip was loaded with aliquots of a sample containing a known peptide molecule of interest. In this evaluation, eight sequential runs of aliquots of the sample were performed: the first run was performed on a fresh, clean chip; and each successive run was performed after applying the regeneration solution as above. In this experiment, a labeled amino acid recognition molecule that binds to a terminal amino acid was used to detect the presence of the peptide of interest. Luminescence of the labeled recognition molecule gives rise to a series of pulses in signal output. The series of pulses provide a characteristic pulsing pattern that is diagnostic of the identity of the corresponding terminal amino acid. This experiment was repeated using two different chips, S42906-16-14 and S42906-16-19.

The workup for the regeneration steps of this experiment is as follows: following the first sequencing run, the chip is cleaned of any residues, and then the regeneration solution is applied to the chip, and the chip is incubated at room temperature for 30 minutes. Lastly, the chip is again cleaned of any residues.

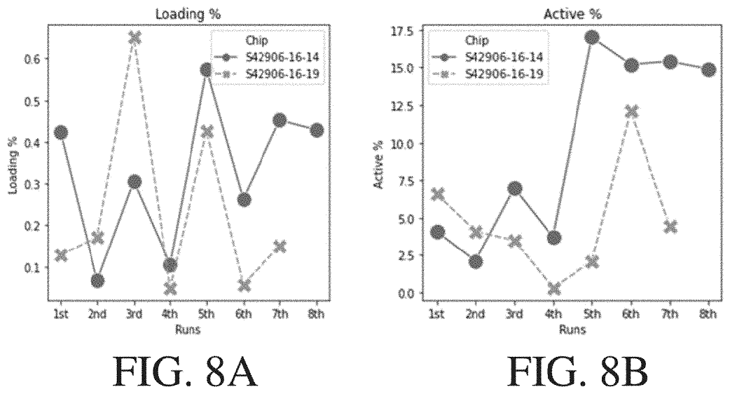

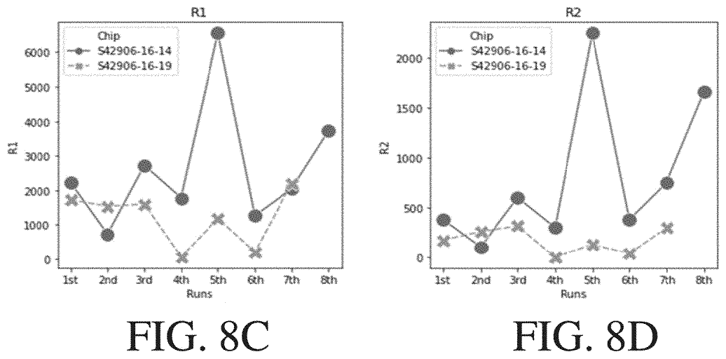

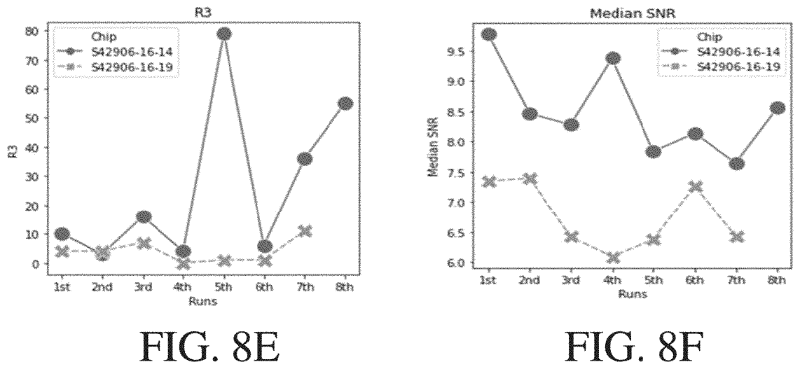

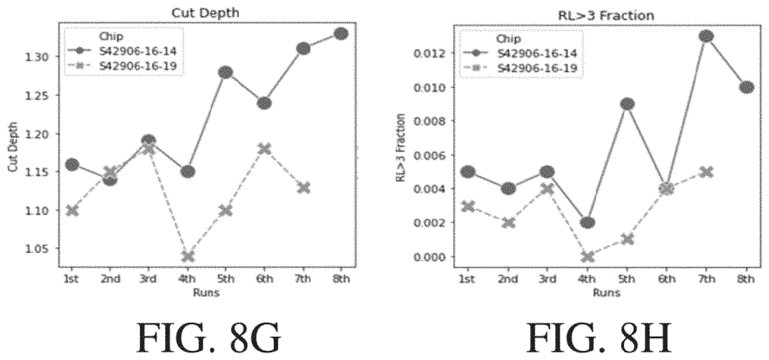

As demonstrated in the signal output results shown in FIGS. 8A-8H, the accuracy and read length results of the peptide molecules for runs beyond the first run exceeded expectations. Percentages of wells loaded for each run are plotted in FIG. 8A. Loading percentages for runs 5 through 8 exceeded 25%. Percentages of actively loaded and pulsing sample wells (active %) of the total number of wells on the chip are plotted in FIG. 8B. This figure indicates between ˜2.4% and 17.5% of samples were actively exhibiting pulses between run 2 and run 8. Between run 5 and run 8, the active % frequency was about 15%.

Accuracy of sequencing results of read lengths 1, 2 and 3 (R1, R2, and R3) are plotted in FIGS. 8C-8E for each of the eight runs. The y-axis reflects a count of sample wells (apertures) that provide a sequencing readout for the read length being plotted. These counts reduce in number as one goes from read length 1 to read length 3. FIG. 8E illustrates a median signal-to-noise ratio (SNR) across each of the eight run. FIG. 8G illustrates an average cut depth across all read lengths. FIG. 8H illustrates a fraction of active wells of the chip for each run having a read length greater than 3.

The results of both experiments indicates that the regeneration process did not generate appreciable interference arising from signals emitted from previously loaded aliquots occurring when sampling subsequently loaded aliquots and thus presents a viable solution for removing samples from the chip.

VIII. Integrated Device Calibration Using Photobleaching Information

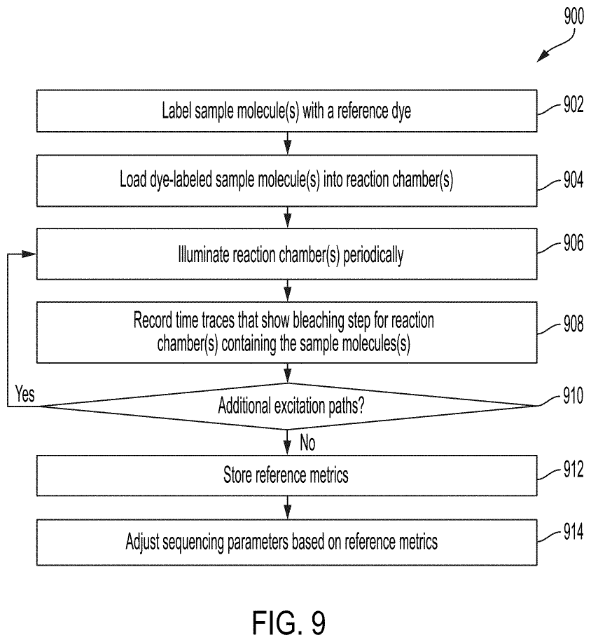

Aspects of the technology described herein further provide for techniques for calibrating an integrated device and/or one or more components that interact with the integrated device using photobleaching information obtained from a reference dye. For example, the integrated device may be part of a system that further comprises at least one excitation source. The calibration techniques described herein may be used to calibrate the system comprising the integrated device. FIG. 9 illustrates an example process 900 for calibrating a system comprising an integrated device using photobleaching information, according to some embodiments.

At act 902, a sample molecule may be labeled with a reference dye. For example, the reference dye may comprise a fluorescent molecule that emits emission light in response to excitation by light from at least one light source. The reference dye molecule may be of a type that binds to a particular sample molecule such that characteristics of the emission light emitted by the reference dye molecule may be used to identify the sample molecule to which the reference dye molecule is bound.

At act 904, the dye-labeled sample is loaded into one or more reaction chambers. For example, in some embodiments, multiple reference dyes of the same type are loaded into a plurality of reaction chambers to evaluate differences in signals received from the plurality of reaction chambers. In some embodiments, the sample molecule may be labeled with the reference dye after the sample molecule is loaded into the reaction chambers, as aspects of the technology described herein are not limited in this respect.

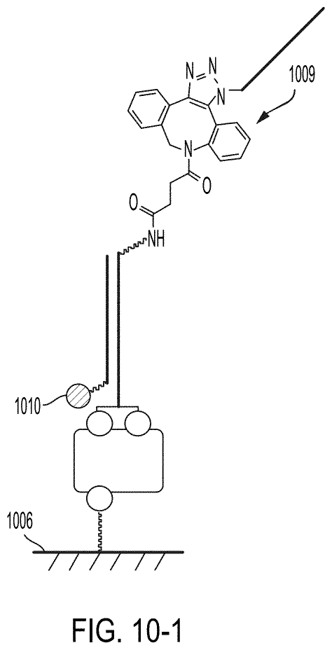

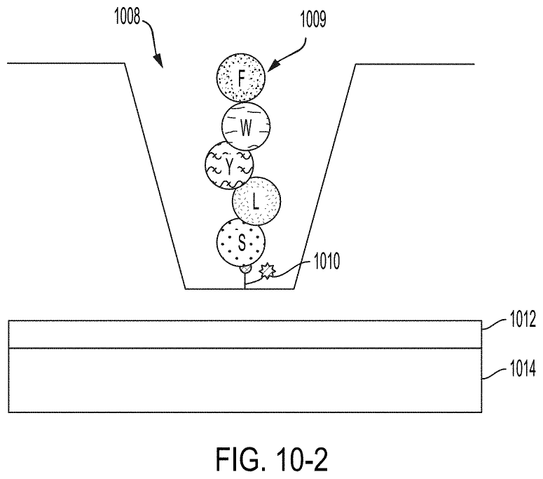

FIG. 10-1 illustrates a sample molecule 1009 labeled with a reference dye 1010 attached to a surface 1006, according to some embodiments. FIG. 10-2 illustrates a dye-labeled sample attached in a reaction chamber, according to some embodiments. In particular, in FIG. 10-2, a sample molecule 1009 comprising a chain of amino acids (phenylalanine (F), tryptophan (W), tyrosine (Y), leucine (L), and serine (S)) is loaded into the reaction chamber 1008. The sample molecule 1009 is labeled with a reference dye molecule 1010. A CMOS chip 1014 comprising photonic components 1012 for collecting signals emitted by the reference dye molecule 1010 may be provided, as described herein with reference to the integrated device overview.

Act 904 may be performed while the reaction chamber(s) is not being illuminated with excitation light. It should be appreciated that, in some embodiments, multiple dye-labeled sample molecules may be loaded into a plurality of reaction chambers at act 904.

In some embodiments, the sample molecule with the reference dye may be loaded into a reaction chamber alone, and a molecule to be sequenced may be loaded into the reaction chamber thereafter. In some embodiments, the molecule with the reference dye may be loaded into the reaction chamber together with the molecule to be sequenced subsequent to performing the calibration process. In some embodiments, the reference dye may be attached to the molecule to be sequenced itself, and the reference dye may be cleaved prior to performing sequencing. In some embodiments, the reference dye may be a freely diffusing dye molecule which need not be attached or otherwise immobilized in the reaction chamber. In some embodiments, the reference dye may be loaded into a reaction chamber as part of the surface chemistry preparation of the chip. Thus, aspects of the technology are not limited to the particular configuration of the reference dye in the reaction chamber. According to some embodiments, act 902 may include techniques are described in U.S. patent application Ser. No. 17/082,906, filed Oct. 28, 2020, titled “METHODS OF PREPARING SAMPLES FOR MULTIPLEX POLYPEPTIDE SEQUENCING” under Attorney Docket Number R0708.70077US01, which is incorporated by reference in its entirety.

At act 906, the reaction chamber(s) is illuminated with excitation light from a light source (e.g., a laser). In particular, at act 906 the reaction chamber(s) may be illuminated with excitation light periodically (e.g., by unblocking the light source, recoupling the light source into the chip, and/or increasing the power delivered by the light source in a time that is fast relative to a characteristic bleaching time of the system). For example, FIG. 11 illustrates an example graph 1100 illustrating light source excitation power over time, according to some embodiments. The excitation light may be delivered to the reaction chambers until the reference dye has been bleached in some embodiments. In some embodiments, delivery of the excitation light to the reaction chambers may be controlled to avoid bleaching the reference dye molecules. Further discussion of photobleaching reference dye molecules is provided herein.

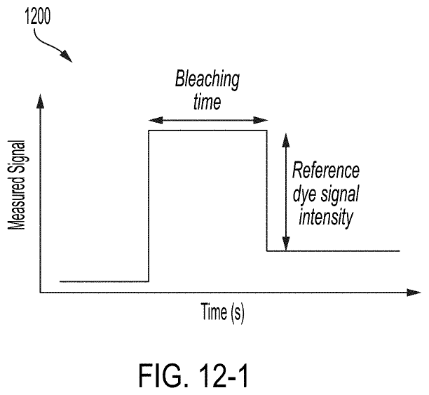

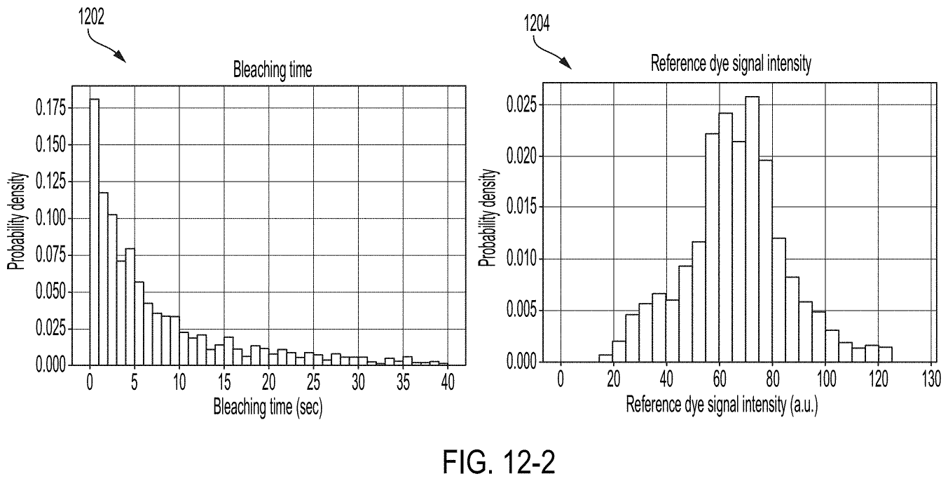

At act 908, time traces are recorded which show the bleaching step for the reaction chambers containing the sample molecule(s) illuminated at act 906. For example, FIG. 12-1 illustrates an example graph 1200 illustrating measured signal from a reference dye over time, according to some embodiments. As shown in FIG. 12-1, the measured signal increases in intensity and subsequently drops in intensity after a period of time. The time period between the measured signal's rise and drop in intensity may be equated to the bleaching time of the reference dye molecule.

At act 910, it is determined whether there are additional excitation paths to excite. If, at act 910, it is determined that there are additional excitation paths to excite, the process 900 returns through the yes branch to act 906 to illuminate additional reaction chambers with the additional excitation paths. If, at act 910, it is determined that there are no additional excitation paths, the process 900 may proceed through the no branch to act 912.

At act 912, reference metrics may be stored. For example, the reference metrics may include the time traces that show a bleaching step obtained at act 908. For example, FIG. 12-2 illustrates example histograms of measured bleaching time and dye intensity for a collection of single molecules in reaction chambers, according to some embodiments. Graph 1902 illustrates a histogram of measured bleaching times for a collection of single molecule reference dyes loaded in the reaction chambers of the integrated device. As shown in FIG. 12-2, the median bleaching time for the collection of single molecule reference dyes is 5.8 seconds. Graph 1904 illustrates a histogram of measured reference dye signal intensity for a collection of single molecule reference dyes loaded in the reaction chambers of the integrated device. In some embodiments, the bleaching times may be determined based at least in part on measured signal intensities. For example, the measured signal intensity may indicate a peak, and the time period between reaching the peak intensity and a drop from the peak intensity may be equated to the bleaching time.

The reference metrics may include information derived from information obtained at act 908. In some embodiments, the reference metrics may include a reference excitation intensity for molecule(s) in a reaction chamber(s) which may be determined based on the characteristic bleaching time. For example, an exponential distribution of bleaching time follows as exp(−t/τ) wherein τ is a characteristic bleaching time. For a collection of single molecules, the median bleaching time divided by n(2) provides an estimate of the characteristic bleaching time τ. A faster characteristic bleaching time may result from a higher excitation intensity, and vice versa.

At act 914, sequencing parameters may be adjusted based on the reference metrics (e.g., characteristic bleaching time, excitation intensity). In some embodiments, the power of a laser delivering excitation light to the reaction chambers may be adjusted. For example, in some embodiments, laser power may be decreased in view of a relatively fast characteristic bleaching time. The laser power may be decreased, in some embodiments, to ensure that excitation intensity during subsequent sequencing applications does not exceed a target value. As such, the read length for sequencing applications may be improved where photodamage may be a factor in the longevity of the sequencing reaction.

In some embodiments, configuration of the integrated device may be adjusted based on the reference metrics. For example, the inventors have recognized that differences in reaction chambers may cause characteristic bleaching times of reference dye to vary between different reaction chambers. As such, the configuration of reaction chambers, photodetectors, and/or a light source delivering excitation light to the pixels may be adjusted based on the reference metrics. For example, for reaction chambers with relatively fast bleaching times, the intensity and/or duration of the excitation light delivered to the reaction chambers may be decreased.

In some embodiments, the bleaching information may be used to identify a number of reference dye molecules in a reaction chamber. For example, in embodiments where excitation light is delivered to the one or more reference dye molecules until the reference dye molecules has photobleached, a bleaching step may be observed by viewing the change in intensity of the signal collected from the reaction chamber (e.g., emitted from one or more reference dye molecules) over time. Photobleaching of one reference dye molecule may be represented by a single step in intensity (e.g., a characteristic drop in the intensity of the signal at a point in time). In some embodiments, the characteristic drop in the intensity of the signal may be substantially equal to the intensity of the signal emitted by the reference dye molecule prior to bleaching. Specifically, after bleaching, the reference dye molecule no longer emits a signal, resulting in the single step decrease in intensity substantially equal to the intensity of the signal emitted by the reference dye molecule prior to bleaching.

If a single bleaching step is observed (e.g., as represented by the change in signal intensity observed over time), it may be determined that a single reference dye molecule has bleached, and therefore that a single reference dye molecule is present in the reaction chamber from which signal is collected. If two or more bleaching steps are observed, it may be determined that multiple reference dye molecules have been loaded into a reaction chamber. The number of reference dye molecules determined to be loaded in the reaction chamber may be considered as a proxy for determining the number of biomolecules of the sample present in the reaction chamber. In some embodiments, the measure of the number of sample molecules in a reaction chamber may be used to include or exclude certain reaction chambers from subsequent analysis. Further description of using photobleaching to determine a measure of quantitative loading is provided herein.

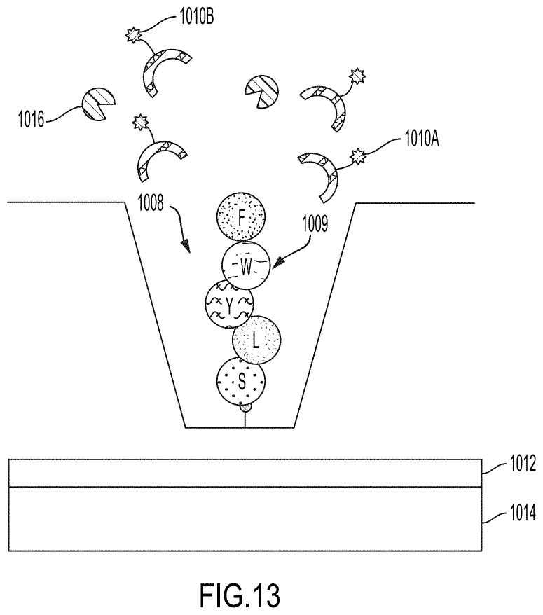

Subsequent to performing the calibration process 900, the integrated device may be used to collect and analyze signals from molecules disposed in the reaction chamber(s). For example, FIG. 13 illustrates a sequencing reaction in a reaction chamber which may be performed subsequent to the calibration process 900, according to some embodiments. The sequencing reaction shown in FIG. 13 may include attachment of reference dye molecules 1010A, 1010B to a sample 1009 in a reaction chamber 1008. As described herein, the reference dye molecules may be of a particular type, for example, binding to a particular type of sample molecule. For example, reference dye molecule 1010A selectively binds only to a first type of amino acids (e.g., Leucine, denoted L) while reference dye molecule 1010B selectively binds to a second type of amino acid (e.g., Phenylalanine, Tyrosine, and Tryptophan, respectively denoted F, Y, and W). Subsequent to sampling the sample molecule 1009 (e.g., by delivering excitation light to the reaction chamber 1008 and collecting signals emitted from the bound reference dye molecule 1010A, 1010B), cleavage of the sample molecule and/or the reference dye molecules 1010A, 1010B bound thereto may be performed with cleaving molecules 1016. The sample molecules may be identified based on signals emitted by the reference molecules. The process of binding, receiving emission signals, and cleaving may be repeated multiple times for a single sample molecule and/or reaction chamber. In some embodiments, the calibration techniques described herein may be used to calibrate a system comprising an integrated device that is configured for use in protein and/or DNA/RNA sequencing applications.

The calibration techniques described herein may be used in combination with any suitable sampling technique to be performed subsequent to calibration. For example, in some embodiments, the calibration techniques described herein may be used in combination with techniques for sample multiplexing as described in in U.S. patent application Ser. No. 17/508,783, filed Oct. 22, 2021, titled “SYSTEMS AND METHODS FOR SAMPLE PROCESS SCALING” under Attorney Docket Number R01408.70112US02, which is incorporated by reference in its entirety.

IX. Quantitative Loading

Some aspects relate to systems and methods for determining a measure of quantitative loading of one or more reaction chambers of the integrated device. For example, the inventors have recognized that it would be advantageous to determine in real-time (e.g., during the loading process) a measure of quantitative loading of one or more reaction chambers of the integrated device. The measure of quantitative loading may comprise an indication of how many biomolecules are present in a reaction chamber (e.g., whether the reaction chamber is empty, singly loaded, doubly loaded, or multi-loaded with more than two biomolecules). In some embodiments, the measure of quantitative loading may comprise an indication of the percentage reaction chambers being singly loaded (or any other degree loaded desired).

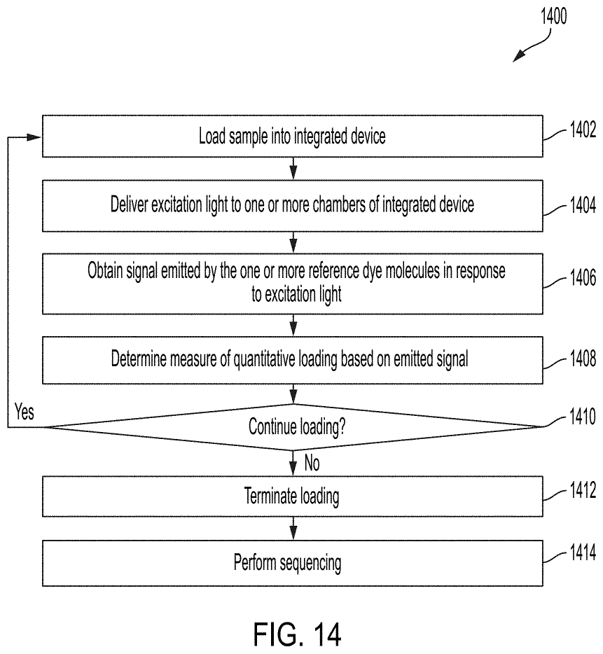

FIG. 14 illustrates an example process 1400 for quantifying loading of one or more reaction chambers, according to some embodiments. The process 1400 may begin at act 1402 where the integrated device is loaded with a sample. The sample may comprise a plurality of biomolecules. The plurality of biomolecules may be of any suitable type. For example, the plurality of biomolecules may comprise biomolecules desired to be identified through sequencing. In some embodiments, the plurality of biomolecules comprise peptides. In some embodiments, the plurality of biomolecules comprise nucleic acids (e.g., ribonucleic acid, deoxyribonucleic acid).

The plurality of biomolecules may be labeled with fluorescent label molecules (also referred to herein as reference dye molecules). In some embodiments, the plurality of biomolecules may be labeled prior to loading the sample onto the integrated device. In some embodiments, the plurality of biomolecules may be unlabeled at the time of loading, and fluorescent label molecules previously or subsequently loaded onto the integrated device may bind to the plurality of biomolecules.

In some embodiments, a fluorescent label molecule may be attached directly to the biomolecule itself. The fluorescent label molecule may be covalently attached to the biomolecule or non-covalently attached to the biomolecule (e.g., via a linker or streptavidin). In some embodiments, the fluorescent label molecule may be loaded into the reaction chamber as part of the surface chemistry preparation of the chip. In some embodiments, the fluorescent label molecule is reversibly bound to the biomolecule, as described further herein. Thus, aspects of the technology are not limited to the particular configuration of the fluorescent label molecule in the reaction chamber. According to some embodiments, act 1402 may include techniques are described in U.S. patent application Ser. No. 17/082,906, filed Oct. 28, 2020, titled “METHODS OF PREPARING SAMPLES FOR MULTIPLEX POLYPEPTIDE SEQUENCING” under Attorney Docket Number R01408.70077US01, which is incorporated by reference in its entirety.

In some embodiments, the fluorescent label molecule comprises an oligonucleotide. The oligonucleotide may be covalently attached to the biomolecule. In some embodiments, the oligonucleotide is hybridized to a complementary oligonucleotide that is covalently attached to the biomolecule.

The fluorescent label molecule may be separated from the biomolecule (e.g., via one or more spacers) by a minimum distance (e.g., more than 1 nm, more than 2 nm, more than 5 nm, 5-10 nm, more than 10 nm, 10-15 nm, more than 15 nm, 15-90 nm, more than 90 nm). Separating the fluorescent label molecule from the biomolecule by a minimum distance may prevent damage occurring to the biomolecule during the quantitative loading determination (e.g., during photobleaching).

At act 1404, excitation light may be delivered to one or more reaction chambers of the integrated device. The excitation light may be generated via at least one excitation source (e.g., a laser), as described herein. The excitation light may be delivered to all of the reaction chambers of the integrated device or a portion thereof.

The excitation light causes any fluorescent label molecules bound to biomolecules in the reaction chambers to excite and emit excitation light. At act 1406, a signal emitted by the one or more reference dye molecules from the respective reaction chambers in response to the excitation light may be obtained.

For example, the emission light may be collected by one or more photodetection regions of the integrated device. In some embodiments, the integrated device may include one photodetection region for each reaction chamber. In some embodiments, multiple photodetection regions may be provided for a single reaction chamber. In some embodiments, multiple reaction chambers may correspond to a single photodetection region.

At act 1408, the measure of quantitative loading may be determined based on the emitted signal obtained at act 1406. As described herein, the measure of quantitative loading may comprise an indication of how many biomolecules are present in a reaction chamber (e.g., whether the reaction chamber is empty, singly loaded, doubly loaded, or multi-loaded with more than two biomolecules). In some embodiments, the measure of quantitative loading may comprise an indication of the percentage reaction chambers being singly loaded (or any other degree loaded desired).

In some embodiments, delivering the excitation light to the one or more reaction chambers of the integrated device comprises photobleaching the one or more fluorescent label molecules, and the measure of quantitative loading is determined based on a number of photobleaching steps represented in the emitted signal (e.g., a number of photobleaching steps that may be observed in the emitted signal). For example, the reaction chamber(s) may be illuminated with excitation light as a step function (e.g., by unblocking the light source, recoupling the light source into the chip, and/or increasing the power delivered by the light source in a time that is fast relative to a characteristic bleaching time of the system). The excitation light may be delivered to the reaction chambers until the reference dye has been bleached. FIGS. 15-1, 15-2, and 15-3 illustrates example traces obtained from chambers having fluorescent label molecules being photobleached at act 1404. In other embodiments, signals emitted by the one or more reference dyes in the reaction chambers may be obtained without performing photobleaching.

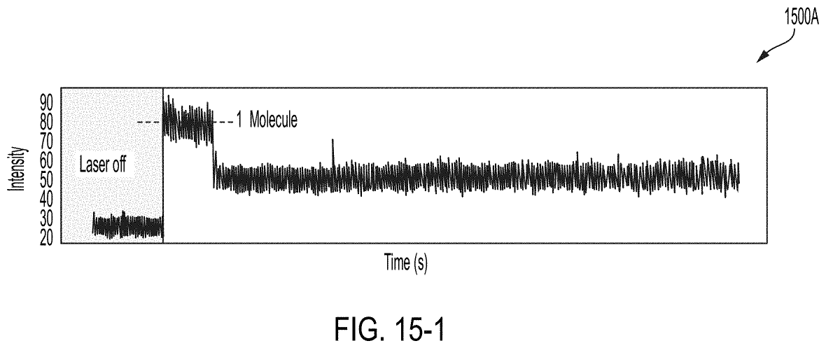

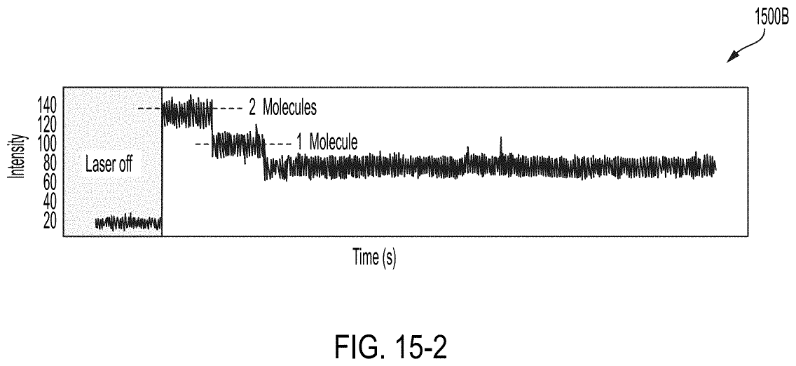

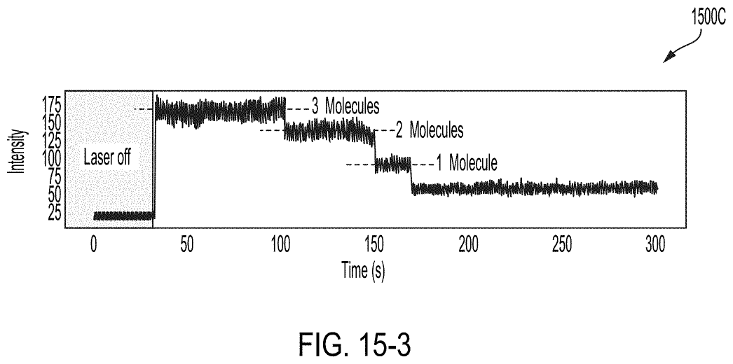

FIG. 15-1 illustrates an example trace 1500A from a chip loading process that represents a single loaded well, according to some embodiments. As shown in FIG. 15-1, signal intensity over a period of time is plotted. When excitation light is first delivered to the reaction chamber, the intensity of a signal received from the reaction chamber may rise to a peak. Subsequent to delivering excitation light to the reaction chambers (e.g., by turning a light source, such as a laser, on), a single step in intensity (e.g., a characteristic drop in the intensity of the signal at a point in time) can be seen in FIG. 15-1. In some embodiments, as depicted in FIG. 15-1, the characteristic drop in the intensity of the signal may be substantially equal to the intensity of the signal emitted by the reference dye molecule prior to bleaching. After bleaching, the reference dye molecule no longer emits a signal, resulting in the single step decrease in intensity shown in FIG. 15-1. Accordingly, the signal shown in FIG. 15-1 indicates the presence of a single fluorescent label in the reaction chamber and therefore a single biomolecule.

FIG. 15-2 illustrates an example trace 1500B from a chip loading process that represents a doubly loaded well, according to some embodiments. As shown in FIG. 15-2, the signal comprises two steps in signal intensity indicating the presence of two fluorescent label molecules and therefore two biomolecules in the reaction chamber.

FIG. 15-3 illustrates an example trace 1500C from a chip loading process that represents a multi-loaded well, according to some embodiments. As shown in FIG. 15-3, the signal comprises three steps in signal intensity (three characteristic drops in intensity of the received signal) indicating the presence of three fluorescent label molecules and therefore three biomolecules in the reaction chamber.

In some instances, no photobleaching steps may be observed. Accordingly, it may be determined, based on the lack of photobleaching observed, that no biomolecules are present in the reaction chamber.

In some embodiments, a signal may be obtained from the reaction chamber without photobleaching fluorescent label molecules present in the reaction chamber. In such embodiments, the number of fluorescent label molecules, and therefore the number of biomolecules, present in the reaction chamber may be determined by a relative intensity of the signal obtained from the reaction chamber.

In some embodiments, the fluorescent label molecule may reversibly bind to the biomolecule or a secondary biomolecule attached there to. For example, the secondary biomolecule may comprise an N-terminal amino acid recognizer (e.g., a ClpS, UBR box, etc.). In some embodiments, the secondary biomolecule is an oligonucleotide. In some embodiments, the oligonucleotide is capable of reversible hybridization to the biomolecule or an oligonucleotide attached thereto.

The binding on and off of the fluorescent label molecule to the biomolecule may be expressed by the on and off pulsing of the fluorescent label molecule. In particular, when the fluorescent label molecule is bound to the biomolecule and excited with excitation light, the fluorescent label molecule emits emission light that may be collected by one or more photodetection regions. When not bound, the fluorescent label molecule may not emit emission light. The fluorescent label molecule may follow a periodic binding pattern to the biomolecule. As such, the fluorescent label molecule generates a periodic pulsing pattern that can be detected by collecting the light emitted from the fluorescent label molecule when bound to a biomolecule.

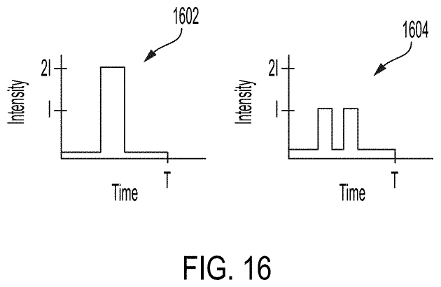

FIG. 16 illustrates a periodic pulsing pattern of fluorescent molecules reversibly bound to a biomolecule, according to some embodiments. Plots 1602 and 1604 illustrate examples of pulses detected by one or more photodetection regions during a single pulsing period T.

Plots 1602 and 1604 illustrate examples of a doubly loaded well having two biomolecules and respective fluorescent labels reversibly bound thereto. Therefore two pulses are detected during the pulsing period T. In the illustrated embodiment of plot 1602, the two fluorescent label molecules bind to the respective biomolecules at substantially the same time during period T. Therefore, a single composite pulse comprised of respective pulses from each of the two fluorescent label molecules is detected in the period T, having intensity 21. In the illustrated embodiment of plot 1604, the two fluorescent label molecules bind to the respective biomolecules at discrete times during period T. Accordingly, two pulses having intensity I are detected in the period T.

Although the illustrated embodiments give examples where a reaction chamber is doubly loaded with two biomolecules, the techniques described herein may likewise be used to detect the presence of a single biomolecule, more than two biomolecules, or the lack of any biomolecules in a reaction chamber by determining the intensity of pulses emitted from the reaction chamber during a pulsing period T. The pulsing period and intensity of the pulses may be determined based on a characteristic pulse width and intensity of the fluorescent label molecule being reversibly bound to the biomolecules.

FIG. 16 illustrates how the timing and intensity of pulses can be detected and used to determine a number of fluorescent label molecules in a reaction chamber being bound to a biomolecule during a respective pulsing period T, and therefore, a number of biomolecules currently present in a reaction chamber. Accordingly, act 1408 of process 1400 may be performed using reversible binding molecules as described herein.

Turning back to process 1400, the process 1400 may proceed, after determining the measure of quantitative loading based on the emitted signal, to act 1410. At act 1410 it is determined whether to continue loading. The determination to continue or terminate loading may be determined based on the measure of quantitative loading determined at act 1410.

For example, in some embodiments, operation of the integrated device may be optimized based on the measure of quantitative loading. In some embodiments, the measure of quantitative loading may be used to adjust how loading of the sample onto the integrated device is performed. In some embodiments, the measure of quantitative loading may be used to determine whether an optimal number of reaction chambers have been loaded with a desired number of biomolecules. In some embodiments, the desired number of biomolecules is one, such that the measure of quantitative loading is used to determine whether an optimal number (e.g., a maximum) of reaction chambers are singly loaded.

If the optimal number of loaded reaction chambers has not been reached, as indicated by the measure of quantitative loading, additional loading may be performed. For example, the process 1400 may loop back to act 1402 where additional sample is loaded onto the integrated device.

In some embodiments, the measure of quantitative loading may be used to adjust an amount of additional sample that is loaded onto the integrated device at subsequent loading steps. For example, the sample may comprise a particular concentration of biomolecules. The measure of quantitative loading may be used to determine whether to increase or decrease the concentration of biomolecules. For example, if it is determined that the optimal number of singly loaded reaction chambers has not been met, additional loading may be performed with an additional sample having a higher concentration of biomolecules than the initial sample.

In some embodiments, the measure of quantitative loading may be used to adjust how signals from the reaction chambers are processed for subsequent analysis. For example, when a reaction chamber is determined to be empty, doubly loaded, or multi-loaded, it may be determined to disregard signals from these reaction chambers either by not performing sequencing on these reaction chambers or not processing signals received from these reaction chambers.

In some embodiments, the measure of quantitative loading may be used to inform future operation of the integrated device. For example, the measure of quantitative loading may be used to adjust how loading of a sample onto the integrated device is performed in the future (e.g., what concentration of biomolecules is present in the sample, how long to perform loading, at what rate to perform loading, etc.).

When it is determined not to continue loading at act 1410, the process 1400 may proceed to act 1412 where loading is terminated. For example, in some embodiments, termination may be performed by removing, washing, and/or replacing of the solution containing non-immobilized (e.g., unbound) peptides. In some embodiments, termination may be performed autonomously.

In some embodiments, terminating loading may include removing the fluorescent label molecule from the reaction chamber. In some embodiments, the fluorescent label molecule may be removed by a chemical cleavage process. In some embodiments, the fluorescent label molecule may be removed by an enzymatic cleavage process

As described herein, the process 1400 may be performed continuously. In some embodiments, the process 1400 may be performed autonomously, for example, using software. This may allow for continuous monitoring of the number of labeled biomolecules in each reaction chamber. A software analysis process may monitor the loading in real time. For example, the software analysis may detect signal pulses/photobleaching steps, as described herein.

In some embodiments, one or more different types of fluorescent label molecules are provided to the biomolecules. For example, a first type of fluorescent label molecule and a second type of fluorescent label molecule may be provided for binding to the biomolecules.

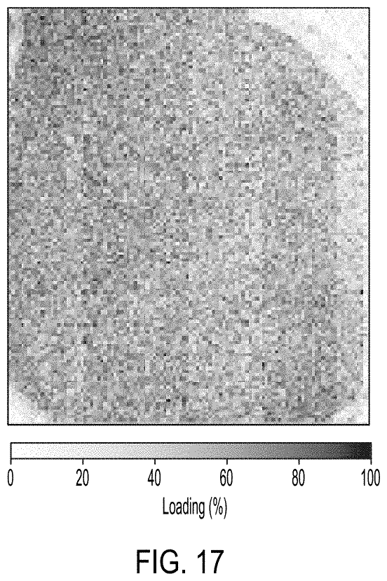

In some embodiments, may generate a “heatmap” of reaction chambers to visually illustrate a percentage of the integrated device (e.g., a percentage of reaction chambers) loaded with one or more biomolecules. FIG. 17 illustrates an example heatmap of reaction chambers illustrating percent loading, according to some embodiments.

In some embodiments, the measure of quantitative loading may only be obtained for a portion of the integrated device (e.g., a portion of all reaction chambers of the integrated device). The measure of quantitative loading may be extrapolated to the remaining reaction chambers of the integrated device to obtain an extrapolated measure of quantitative loading for the entire integrated device (or other portions therefore).

In some embodiments, sequencing of the sample may be performed subsequent to performing the calibration and quantitative loading techniques described herein (e.g., at act 1414 of process 1400). For example, the calibration and quantitative loading techniques may be used in combination with techniques for sample identification using machine learning, for example, as described in in U.S. patent application Ser. No. 16/900,582, filed Jun. 12, 2020, titled “TECHNIQUES FOR PROTEIN IDENTIFICATION USING MACHINE LEARNING AND RELATED SYSTEMS AND METHODS” under Attorney Docket Number R0708.70063US01, which is incorporated by reference in its entirety.

The techniques described herein for obtaining real-time measurements relating to the integrated device (e.g., to the occupancy of one or more reaction chambers of the integrated device) may be implemented at any time during use of the integrated device (e.g., during sample preparation, during loading, during sequencing, during sample removal, etc.), and such techniques are not limited to use during loading of the integrated device.

X. Equivalents and Scope

Having thus described several aspects and embodiments of the technology of the present disclosure, it is to be appreciated that various alterations, modifications, and improvements will readily occur to those of ordinary skill in the art. Such alterations, modifications, and improvements are intended to be within the spirit and scope of the technology described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. The transitional phrases “consisting of” and “consisting essentially of” shall be closed or semi-closed transitional phrases, respectively.

What is claimed is:

1. A method for reusing an integrated device to process a sample, the sample being divided into a plurality of aliquots, wherein the sample comprises analytes and the analytes comprise a biomolecule comprising one or more luminescently labeled molecules, the method comprising:
loading a first aliquot of the plurality of aliquots into at least some of a plurality of chambers of the integrated device;
sampling analytes of the first aliquot while the analytes are present in the at least some of the plurality of chambers, wherein the step of sampling comprises determining a luminescent lifetime, a luminescent intensity, a wavelength, a pulse duration, and/or an interpulse duration of a signal emitted by the one or more luminescently labeled molecules;
removing the first aliquot from the at least some of the plurality of chambers of the integrated device; and
loading a second aliquot of the plurality of aliquots into the at least some of the plurality of chambers of the integrated device.

2. The method of claim 1, further comprising sampling analytes of the second aliquot while the analytes of the second aliquot are present in the at least some of the plurality of chambers.

3. The method of claim 1, wherein removing the first aliquot from the at least some of the plurality of chambers comprises:
disrupting a covalent bond between two or more coating molecules bound to a surface of the plurality of chambers and coupling moieties, the analytes of the first aliquot being bound to the coupling moieties.

4. The method of claim 3, wherein the dissociating is performed by contacting the surface of the plurality of chambers with a solution for a discrete period of time.

5. The method of claim 4, wherein the step of dissociating is performed at a temperature greater than or equal to 20 degrees Celsius and less than or equal to 22 degrees Celsius.

6. The method of claim 4, wherein the discrete period of time comprises no more than 60 minutes.

7. The method of claim 4, wherein the solution comprises ammonium acetate, water, and hexafluoro-2-propanol.

8. The method of claim 4, wherein the solution further comprises free Biotin and/or one or more analogs thereof.

9. The method of claim 1, wherein the step of removing the first aliquot from the at least some of the plurality of chambers comprises enzymatically digesting the analytes of the first aliquot in the at least some of the plurality of chambers.

10. The method of claim 1, wherein the analytes of the first aliquot are bound to coupling moieties and removing the first aliquot from the at least some of the plurality of chambers comprises disrupting a covalent linker between the coupling moieties and the analytes of the first aliquot.

11. A method for reusing an integrated device to process a sample, the sample comprising analytes, the analytes comprising a biomolecule comprising one or more luminescently labeled molecules, the method comprising:
loading at least a portion of the sample into a plurality of chambers of the integrated device;
sampling analytes of the at least the portion of the sample while the analytes are present in the plurality of chambers, wherein the step of sampling comprises collecting a signal emitted by the one or more luminescently labeled molecules indicative of a luminescent lifetime, a luminescent intensity, a wavelength, a pulse duration, and/or an interpulse duration; and
removing the at least the portion of the sample from the plurality of chambers, wherein the removing comprises:
disrupting a covalent bond between respective coating molecules bound to a surface of the plurality of chambers and coupling moieties, the analytes of the at least the portion of the sample being bound to the coupling moieties.

12. The method of claim 11, wherein the covalent bond binds the analytes to the surface of the plurality of chambers and disrupting the bond releases the analytes from the surface of the plurality of chambers.

13. The method of claim 12, wherein the removing further comprises, subsequent to the disrupting, flushing the analytes from the plurality of chambers.

14. The method of claim 11, wherein the disrupting is performed by contacting the surface of the plurality of chambers with a solution for a discrete period of time.

15. The method of claim 14, wherein the disrupting is performed at a temperature greater than or equal to 20 degrees Celsius and less than or equal to 22 degrees Celsius.

16. The method of claim 14, wherein the discrete period of time comprises no more than 60 minutes.

17. The method of claim 11, wherein the coating molecules comprise biotin and the coupling moieties comprise streptavidin.

18. The method of claim 8, wherein the solution comprises ammonium acetate and water.

19. The method of claim 18, wherein the solution further comprises hexafluoro-2-propanol.

20. A method for determining whether a sample is present in one or more chambers of an integrated device, the method comprising:
loading at least a portion of the sample into the one or more chambers of the integrated device;
removing the at least the portion of the sample from the one or more chambers of the integrated device;
delivering excitation light to the one or more chambers of the integrated device;
collecting signals emitted from the plurality of chambers in response to the excitation light at a photodetection region of the integrated device; and
determining, based on the signals, whether at least some of the at least the portion of the sample is present in the one or more chambers of the integrated device.
